(19) United States
(12) Patent Application Publication
(10) Pub. No.: US 2024/0165163 A1
(43) Pub. Date: May 23, 2024

(54) HYBRID RECEPTORS WITH MULTIPLE TRANSCRIPTIONAL REGULATORS

(71) Applicants: Axel Hyrenius WITTSTEN, San Francisco, CA (US); The Regents of the University of California, Oakland, CA (US)

(72) Inventors: Kole T. ROYBAL, San Francisco, CA (US); Julie GARCIA, San Francisco, CA (US); Iowis ZHU, San Francisco, CA (US); Raymond LIU, San Francisco, CA (US); Axel Hyrenius WITTSTEN, San Francisco, CA (US)

(21) Appl. No.: 18/551,931

(22) PCT Filed: Mar. 23, 2022
(86) PCT No.: PCT/US22/21605

§ 371 Date: Sep. 22, 2023

Related U.S. Application Data
(60) Provisional application No. 63/165,428, filed on Mar. 24, 2021.

(51) Int. Cl. A61K 35/17; A61K 39/00; C07K 16/28; C07K 16/32; C07K 16/40; C12N 15/86

(52) U.S. Cl. CPC A61K 35/17; A61K 39/4611; C07K 16/2803; C07K 16/2878; C07K 16/32; C07K 16/40; C12N 15/86; A61K 2239/13; C07K 2317/53; C07K 2317/622; C07K 2319/03; C07K 2319/50; C12N 2740/15043

(57) ABSTRACT

The present disclosure relates generally to the field of immunology, and particularly relates to hybrid chimeric antigen receptors designed to combine fast time-scale intracellular signal transduction and long time-scale transcription regulation. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

### CROSS REFERENCE TO RELATED APPLICATIONS

This application claims the benefit of U.S. Provisional Application No. 63/165,428, filed Mar. 24, 2021, which is incorporated herein by reference in entirety and for all purposes.

### STATEMENT REGARDING FEDERALLY SPONSORED R&D

This invention was made with government support under grant no. OD025751 awarded by The National Institutes of Health. The government has certain rights in the invention.

### FIELD

The present disclosure relates generally to the field of immunology, and particularly relates to hybrid chimeric antigen receptors designed to combine fast time-scale intracellular signal transduction and long time-scale transcription regulation. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

### BACKGROUND

Notch receptors are transmembrane proteins that mediate cell-cell contact signaling and play a central role in development and other aspects of cell-to-cell communication. Notch receptors are involved in and are required for a variety of cellular functions during development, and are important for the function of a vast number of cell-types across species.

A number of existing first-generation synthetic derivatives of Notch receptors, which are often referred to as “SynNotch” have been developed recently by replacing the extracellular ligand-binding domain, which in wild-type Notch contains multiple EGF-like repeats, with an antibody derivative, and replacing the cytoplasmic domain with a transcription activator of choice, but still relying on the Notch NRR (L. Morsut et al., Cell (2016) 164:780-91) and the standard two-step proteolysis. However, the NRR spans approximately 160 amino acids, making this domain alone about three times the size of some mature proteins, such as insulin or epidermal growth factor (EGF). This makes expression of the receptor less efficient, and can exceed the capacity of some widely used cloning and transfection vectors.

In addition, these first-generation SynNotch and the second-generation SynNotch receptors, in contrast to chimeric antigen receptor (CARs), do not elicit membrane proximal signaling via kinase cascades. The receptors, instead, translate ligand-binding to release of a receptor-tethered transcription factor that shuttles to the nucleus to regulate a user-defined transcriptional circuit.

In particular, these receptors lack the ability to initiate fast time-scale signaling that regulates cellular processes such as metabolic reprogramming, proliferation, growth factor production, or cytotoxicity.

The present disclosure provides, among other things, a new class of hybrid SynNotch receptors that incorporate intracellular signaling domains (e.g. stimulation domains and co-stimulation domains of a CAR, for example, co-stimulation domains from 4-1BB, CD28, and a cytoplasmic tail of CD3zeta, etc.) that can initiate activation of T cells concomitant with custom transcriptional regulation.

### SUMMARY

Provided herein, among others, includes a chimeric receptor comprising, from N-terminus to C-terminus: a) an extracellular ligand-binding domain having a binding affinity for a selected ligand; b) a linking polypeptide; c) a transmembrane domain (TMD) comprising one or more ligand-inducible proteolytic cleavage sites; and d) an intracellular domain (ICD). In some embodiments, the ICD comprises, in any order: (i) an intracellular signaling domain (SD) comprising at least one costimulatory domain derived from a signaling molecule and an activation domain, and (ii) a transcriptional regulator. In certain embodiments, binding of the selected ligand to the extracellular ligand-binding domain induces cleavage at a ligand-inducible proteolytic cleavage site disposed between the ICD and the linking polypeptide. In other embodiments, binding of the selected ligand to the extracellular ligand-binding domain induces proximal signaling cascades through the intracellular SD. In some embodiments, the chimeric receptor does not comprise a LIN-12-Notch repeat (LNR) and/or a heterodimerization domain (HD) of a Notch receptor.

In some embodiments, the extracellular domain comprises an antigen-binding moiety capable of binding to a ligand on the surface of a cell. In some embodiments, the cell is a pathogenic cell. In some embodiments, the cell is a human cell. In some embodiments, the human cell is a tumor cell. In some embodiments, the human cell is a terminally differentiated cell.

In some embodiments, the ligand comprises a protein or a carbohydrate. In certain embodiments, the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B-cell maturation antigen (BCMA), green fluorescent protein (GFP), blue fluorescent protein (BFP) enhanced green fluorescent protein (EGFP), and signal regulatory protein α (SIRPα).

In some embodiments, the ligand is selected from cell surface receptors, adhesion proteins, integrins, mucins, lectins, tumor-associated antigens, and tumor-specific antigens. In some embodiments, the ligand is a tumor-associated antigen or a tumor-specific antigen. In some embodiments, the extracellular ligand-binding domain comprises the ligand-binding portion of a receptor. In some embodiments, the antigen-binding moiety is selected from the group consisting of an antibody, a nanobody, a diabody, a triabody, a minibody, an F(ab′)2 fragment, an F(ab)v fragment, a single chain variable fragment (scFv), a single domain antibody (sdAb), and a functional fragment thereof. In some exemplary embodiments, the antigen-binding moiety comprises an scFv.

In certain embodiments, the antigen-binding moiety specifically binds to a tumor-associated antigen selected from the group consisting of CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD117), MUC1, NCAM, PAP, PDGFR-β, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl. In some exemplary embodiments, the tumor-associated antigen is CD19, BCMA, CEA, HER2, MUC1, CD20, ALPPL2, SIRPα, or EGFR.

In other exemplary embodiments, the tumor-associated antigen is CD19, BCMA, HER2, or ALPPL2.

In some embodiments, the linking polypeptide of the chimeric receptor provided herein comprises a hinge domain. In some embodiments, the hinge domain is capable of promoting oligomer formation of the chimeric polypeptide via intermolecular disulfide bonding.

In some embodiments, the hinge domain is derived from a CD8α hinge domain, a CD28 hinge domain, a CD152 hinge domain, a PD-1 hinge domain, a CTLA4 hinge domain, an OX40 hinge domain, an IgG1 hinge domain, an IgG2 hinge domain, an IgG3 hinge domain, and an IgG4 hinge domain, or a functional variant of any thereof. In certain exemplary embodiments, the linking polypeptide is derived from the group selected from: a CD8α hinge domain or a functional variant thereof, a CD28 hinge domain or a functional variant thereof, OX40 hinge domain or a functional variant thereof, and an IgG4 hinge domain or a functional variant thereof.

In some embodiments, the linking polypeptide is derived from a CD8α hinge domain or a functional variant thereof. In other embodiments, the linking polypeptide is derived from an CD28 hinge domain or a functional variant thereof. In some specific embodiments, the linking polypeptide comprises an amino acid sequence having at least 80% sequence identity to SEQ ID NO: 3.

In some embodiments, the one or more ligand-inducible proteolytic cleavage sites comprises a γ secretase cleavage site. In some embodiments, the TMD comprises an amino acid sequence having at least 80% sequence identity to SEQ ID NO: 4. In certain embodiments, the chimeric receptor of the present disclosure further comprises a stop-transfer-sequence (STS) positioned between the TMD and the ICD. In some exemplary embodiments, the stop-transfer-sequence comprises an amino acid sequence having at least 80% sequence identity to SEQ ID NO: 5.

In some embodiments, the signaling molecule comprises a class 1 or a class 3 human membrane protein. In certain embodiments, the signaling molecule is selected from the group consisting of CD28, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, 4-1BB, OX40, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-PIA, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC. In some exemplary embodiments, the signaling molecule is selected from the list consisting of OX40, ICOS, 4-1BB, CTLA4, CD28, CD30, CD2, CD27, and CD226.

In some embodiments, the activation domain comprises one or more immunoreceptor tyrosine-based activation motifs (ITAMs). In some embodiments, the one or more ITAMs are derived from CD3ζ, CD3σ, CD3/, and CD3ε. In certain embodiments, the one or more ITAMs have at least about 80, 85, 90, 95, 96, 97, 98, 99, or 100% sequence identity to a CD3ζ ITAM.

| In some embodiments, the transcriptional regulator comprises a transcriptional activator or a transcriptional repressor. In some embodiments, the transcriptional regulator further comprises a nuclear localization sequence (NLS) derived from a protein selected from the group consisting of Gal4, tetR, ZFHD1, and HAP1, and wherein the transcriptional regulator comprises a transactivation domain derived from a protein selected from the group consisting of VP64, VP65, KRAB, and VP16.

In certain embodiments, the chimeric receptor provided herein comprises an amino acid sequence having at least 80% sequence identity to any one of SEQ ID NOS: 15-31 and 34-45.

In certain embodiments, the chimeric receptor provided herein further comprises a signal sequence, a detectable label, a tumor-specific cleavage site, a disease-specific cleavage site, or a combination thereof.

In other aspects, the present disclosure also includes a recombinant nucleic acid comprising a nucleotide sequence encoding the chimeric receptor described herein. In certain embodiments, the nucleotide sequence is incorporated into an expression cassette or an expression vector. In certain embodiments, the expression vector is a viral vector. In certain embodiments, the viral vector is a lentiviral vector, an adeno virus vector, an adeno-associated virus vector, or a retroviral vector.

Further, the present disclosure includes a recombinant cell comprising the chimeric receptor and/or the recombinant nucleic acid described herein. In some embodiments, the recombinant cell is a eukaryotic cell. In some embodiments, the eukaryotic cell is a mammalian cell. In some embodiments, the mammalian cell is an immune cell, a neuron, an epithelial cell, and endothelial cell, or a stem cell. In some embodiments, the immune cell is a B cell, a monocyte, a natural killer cell, a basophil, an eosinophil, a neutrophil, a dendritic cell, a macrophage, a regulatory T cell, a helper T cell, a cytotoxic T cell, or other T cell.

In some embodiments, the recombinant cell of the present disclosure comprises: a) a first chimeric receptor and a second chimeric receptor described herein; and/or b) a first nucleic acid and a second nucleic acid described herein. In some embodiments, the first chimeric receptor and the second chimeric receptor do not have the same sequence. In other embodiments, the first nucleic acid or the second nucleic acid do not have the same sequence. In some embodiments, the first chimeric receptor modulates the expression and/or activity of the second chimeric receptor.

In some embodiments, the recombinant cell of the present disclosure further comprises an expression cassette encoding a protein operably linked to a promoter, wherein expression of the protein is modulated by the transcriptional regulator. In some embodiments, the protein is heterologous to the cell. In some embodiments, the promoter is a yeast GAL4 promoter. In some embodiments, the protein is a cytokine, a cytotoxin, a chemokine, an immunomodulator, a pro-apoptotic factor, an anti-apoptotic factor, a hormone, a differentiation factor, a de-differentiation factor, an immune cell receptor (e.g., a TCR or CAR), or a reporter.

Further provided herein includes a method for making the recombinant cell described herein, comprising: a) providing a cell capable of protein expression; and b) contacting the provided cell with a recombinant nucleic acid described herein into the provided cell. In some embodiments, the cell is obtained by leukapheresis performed on a sample obtained from a subject, and the cell is contacted ex vivo. In some embodiments, the recombinant nucleic acid is encapsulated in a viral capsid or a lipid nanoparticle.

Another aspect of the present disclosure relates to a pharmaceutical composition comprising a pharmaceutically acceptable carrier, and one or more of the following: a) the recombinant nucleic acid described herein; and b) the recombinant cell described herein. In some embodiments, the composition comprises a recombinant nucleic acid described herein and a pharmaceutically acceptable carrier. In certain embodiments, the recombinant nucleic acid is encapsulated in a viral capsid or a lipid nanoparticle.

Further provided herein, among others, includes a system for modulating an activity of a cell, inhibiting a target cancer cell, or treating a health condition in an individual in need thereof. In some embodiments, the system comprises one or more of the following: a) a chimeric receptor described herein; b) a recombinant nucleic acid described herein; c) a recombinant cell described herein; and d) a pharmaceutical composition described herein.

In other aspects, the present disclosure also provided a method for modulating an activity of a cell, comprising: a) providing a recombinant cell described herein; and b) contacting the recombinant cell with a selected ligand, wherein binding of the selected ligand to the extracellular ligand-binding domain induces cleavage of a ligand-inducible proteolytic cleavage site and releases the transcriptional regulator, wherein the released transcriptional regulator modulates an activity of the recombinant cell.

In some embodiments, the contacting is carried out in vivo, ex vivo, or in vitro.

In some embodiments, the activity of the cell to be modulated is selected from the group consisting of: expression of a selected gene, proliferation, apoptosis, non-apoptotic death, differentiation, dedifferentiation, migration, secretion of a molecule, cellular adhesion, and cytolytic activity. In some embodiments, the released transcriptional regulator modulates expression of a gene product of the cell.

In some embodiments, the released transcriptional regulator modulates expression of a heterologous gene product. In some embodiments, the gene product of the cell is selected from the group consisting of chemokine, a chemokine receptor, a chimeric antigen receptor, a cytokine, a cytokine receptor, a differentiation factor, a growth factor, a growth factor receptor, a hormone, a metabolic enzyme, a pathogen-derived protein, a proliferation inducer, a receptor, an RNA guided nuclease, a site-specific nuclease, a T cell receptor, a toxin, a toxin derived protein, a transcriptional regulator, a transcriptional activator, a transcriptional repressor, a translational regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

In some embodiments, the released transcriptional regulator modulates differentiation of the cell, and wherein the cell is an immune cell, a stem cell, a progenitor cell, or a precursor cell.

Further provided herein, among others, includes a method for inhibiting an activity of a target cell in an individual, the method comprising administering to the individual an effective number of the recombinant cells described herein. In some embodiments, the recombinant cells inhibit an activity of the target cell in the individual.

In some embodiments, the target cell is a pathogenic cell. In some embodiments, the pathogenic cell is a cancer cell. In some embodiments, the target cell is an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-Cell lymphoma cell, a renal cancer cell, a sarcoma cell, a stomach cancer cell, a carcinoma cell, a mesothelioma cell, or a sarcoma cell.

In other aspects, the present disclosure provides a method for the treatment of a health condition in an individual in need thereof, the method comprising administering to the individual a first therapy comprising an effective number of the recombinant cell described herein, wherein the recombinant cell treats the health condition in the individual.

In some embodiments, the method for the treatment of a health condition in an individual in need thereof further comprises administering to the individual a second therapy.

In some embodiments, the second therapy is selected from the group consisting of chemotherapy, radiotherapy, immunotherapy, hormonal therapy, and toxin therapy.

In some embodiments, the first therapy and the second therapy are administered together in the same composition or in separate compositions. In some embodiments, the first therapy and the second therapy are administered at the same time. In other embodiments, the first therapy and the second therapy are administered sequentially. In certain embodiments, the first therapy is administered before the second therapy. In other embodiments, the first therapy is administered after the second therapy. In yet other embodiments, the first therapy and the second therapy are administered in rotation.

The present disclosure also provides the use of one or more of the following for the treatment of a health condition: a) a chimeric receptor described herein; b) a recombinant nucleic acid described herein; c) a recombinant cell described herein; and d) a composition described herein. In some embodiments, the present disclosure relates to the use of any of the forgoing for the manufacture of a medicament for the treatment of a health condition. In some embodiments, the health condition is cancer. In certain embodiments, the cancer is a solid tumor, a soft tissue tumor, or a metastatic lesion.

### BRIEF DESCRIPTION OF THE DRAWINGS

FIGS. 1A-1C illustrate the design of exemplary hybrid SynNotch CARs in accordance with some embodiments of the disclosure and predicted function of hybrid SynNotch CAR circuits. FIG. 1A is a diagram of all possible intracellular domain configurations. FIG. 1B is a detailed diagram of Hybrid SynNotch CAR domains. FIG. 1C shows hypothesized short term proximal and long term transcriptional signaling induced by Hybrid SynNotch CARs.

FIGS. 2A-2D schematically summarize the results from experiments performed to illustrate the expression of various exemplary hybrid SynNotch CARs and circuit induction. Primary human T-cells were activated with anti-CD3/anti-CD28 Dynabeads (Gibco) and transduced with two lentiviral constructs expressing either a receptor or a transcriptional reporter construct. Hybrid SynNotch CAR with 41BB costimulatory domains (FIG. 2A) or CD28 costimulatory domains (FIG. 2C) were sorted on Day 5 post initial T-cell stimulation, purifying the receptor and reporter dual positive population. To assess circuit induction, on Day 14 post initial T cell stimulation, T-cells expressing anti-CD19 receptors with 41BB costimulatory domains (FIG. 2B) or CD28 costimulatory domains (FIG. 2D) and the BFP reporter were co-cultured with K562 cells (blue), or CD19+ K562 cells (red) for 48 hours. Transcriptional activation of the inducible BFP reporter gene was subsequently measured using a Fortessa X-50 (BD).

FIGS. 3A-3B illustrate the hybrid SynNotch CAR activation marker expression. T-cells expressing anti-CD19 receptors with 41BB costimulatory domains (FIG. 3A) or CD28 costimulatory domains (FIG. 3B) and the BFP reporter were produced as described in FIG. 2. Transduced cells were co-cultured with K562 cells (gray), or CD19+ K562 cells (blue or red) for 48 hours. Expression of activation markers CD25, CD39, CD69 and PD-1 were subsequently measured using a Fortessa X-50 (BD).

FIG. 4 schematically summarizes the results from experiments performed to illustrate the proliferation of exemplary hybrid SynNotch CARs. T-cells expressing anti-CD19 receptors with 41BB costimulatory domains or CD28 costimulatory domains and the BFP reporter were produced as described in FIG. 2. Transduced cells were stained with Cell Trace Far Red (CTFR), then co-cultured with K562 cells (gray), or CD19+ K562 cells (blue or red) for 5 days. Dilution of CTFR dye was subsequently measured using a Fortessa X-50 (BD).

FIGS. 5A-5B summarize the results from experiments performed to illustrate the cytokine secretion by exemplary hybrid SynNotch CARs. T-cells expressing anti-CD19 receptors with 41BB costimulatory domains (FIG. 5A) or CD28 costimulatory domains (FIG. 5B) and the BFP reporter were produced as described in FIG. 2. Transduced cells were co-cultured with K562 cells (gray), or CD19+ K562 cells (blue or red). After 48 hours, Brefeldin A, Monesin and a second bolus of K562 cells (either with or without CD19+ expression) was added to the co-cultures. Co-cultures were incubated for an additional 6 hours, then transduced cells were assessed using a Fortessa X50 (BD) for intracellular expression of the cytokines Granzyme B, IFNy, IL-2 and TNFa. Note that data was not collected for the Hybrid SynNotch CAR with ICD CD28-CD33ζ-Gal4VP64.

FIGS. 6A-6B summarize the results from experiments performed to illustrate the target killing by exemplary hybrid SynNotch CARs. T-cells expressing anti-CD19 receptors with 41BB costimulatory domains (FIG. 6A) or CD28 costimulatory domains (FIG. 6B) and the BFP reporter were produced as described in FIG. 2. A549 cells expressing the CD19 ligand and the nuclear stain mkate2 were allowed to adhere to a 96 well flat bottom plate for 24 hours, then transduced T cells were added at a 1:1 ratio. The plate was incubated in an Incucyte, which captured plate images and fluorescence every 2 hours for 5 days. Imaging software was used to calculate the number of A549 CD19+ mkate2+ cells in culture at each timepoint. For each experimental group, the A549 cell count was normalized to that of the Hinge Notch experimental group.

FIGS. 7A-7C illustrate the in vivo efficacy of the hybrid SynNotch CARs. FIG. 7A is a description of experimental timeline. NOD.Cg-Prkde^{scid}Il2rgtm1^{Wjl/SzJ} (NSG) mice were dosed intravenously with 0.5×106 Nalm6-Luc-GFP tumor cells. Bulk CD3+ T-cells were co-transduced with the anti-CD19 Hybrid SynNotch CAR with ICD CD3ζ-Gal4VP64-CD28 and the BFP reporter as described in FIG. 2. 0.5×106 transduced CD3+ T cells were dosed to animals via retro-orbital injection 4 days post tumor injection. FIG. 7B shows tumor burden as measured via bioluminescence imaging of luciferase secreting tumor cells using an IVIS Spectrum. FIG. 7C shows a survival curve.

FIGS. 8A-8D illustrate that minimized 4-1BB variants improve NF-kB signaling and reduce noise. FIG. 8A is a description of 41BB variants, depicting the amino acids deleted to create the “no STS” and “trunc” 41BB costimulatory domains. FIG. 8B shows T cells were co-transduced to express the anti-CD19 Hybrid SynNotch CAR with 41BB variants and the BFP reporter as described in FIG. 2. FIG. 8C shows circuit induction was assessed as described in FIG. 2. FIG. 8D shows a Jurkat cell line transduced to express an mCherry reporter under a common promoter for NfKB. This NfKB reporter cell line was then transduced with anti-CD19 Hybrid SynNotch CARs, and co-cultured with K562 cells expressing CD19. mCherry expression was assessed as a proxy for NfKB activity at 24, 48 and 72 hours post co-culture via flow cytometry.

FIGS. 9A-9D shows further iterations on minimized 41BB variants. FIG. 9A is a description of 41BB variants, depicting the amino acids deleted to create the “min” 41BB costimulatory domains, and amino acid regions duplicated to create the “trunc41BBtrunc41BB” costimulatory domain. FIG. 9B shows T cells were co-transduced to express the anti-CD19 Hybrid SynNotch CAR with 41BB variants and the BFP reporter as described in FIG. 2. FIG. 9C shows circuit induction was assessed as described in FIG. 2. FIG. 9D shows a Jurkat cell line was transduced to express an mCherry reporter under a common promoter for NfKB. This NfKB reporter cell line was then transduced with anti-CD19 Hybrid SynNotch CARs, and co-cultured with K562 cells expressing CD19. mCherry expression was assessed as a proxy for NfKB activity at 24, 48 and 72 hours post co-culture via flow cytometry.

FIGS. 10A-10B show Trunc41BB Hybrid SynNotch CAR in vivo efficacy. FIG. 10A shows a description of experimental timeline. NOD.Cg-Prkdc^{scid}Il2rgtm1^{Wjl/SzJ} (NSG) mice were dosed subcutaneously with 4×10⁶ CD19 ligand expressing M28 tumor cells. Bulk CD3+ T-cells were co-transduced with the anti-CD19 Hybrid SynNotch CAR with ICDs as indicated in 10B and the BFP reporter as described in FIG. 2. 6×10⁶ transduced CD3+ T cells were dosed to animals via retro-orbital injection 7 days post tumor injection. FIG. 10B shows tumor volume assessed via caliper measurements weekly.

FIGS. 11A-11C show minimized CD28 variants reduce noise. FIG. 11A is a description of CD28 variants, depicting the amino acids deleted to create the “no STS” and “trunc” CD28 costimulatory domains. FIG. 11B shows T cells co-transduced to express the anti-CD19 Hybrid SynNotch CAR with CD28 variants and the BFP reporter as described in FIG. 2. FIG. 11C shows circuit induction assessed as described in FIG. 2.

FIGS. 12A-12C show further iterations on minimized CD28 variants. FIG. 12A is a description of CD28 variants, depicting the amino acids deleted to create the “CD28ΔTPRRP,” “truncCD28ΔTPRRP” and “fullytruncCD28” costimulatory domains. FIG. 12B shows T cells co-transduced to express the anti-CD19 Hybrid SynNotch CAR with CD28 variants and the BFP reporter as described in FIG. 2. FIG. 12C shows circuit induction assessed as described in FIG. 2.

FIGS. 13A-13C show “Third Generation” variants. FIG. 13A is a description of “third generation” variants, which include one of the CD28 signaling motifs appended to the C terminus of a trunc41BB costimulatory domain. FIG. 13B shows T cells co-transduced to express the anti-CD19 Hybrid SynNotch CAR with third generation variants and the BFP reporter as described in FIG. 2. FIG. 13C shows circuit induction assessed as described in FIG. 2.

FIGS. 14A-14B show Trunc41BB Hybrid SynNotch CAR In Vivo Efficacy. FIG. 14A is a description of experimental timeline. NOD.Cg-Prkdc^{scid}Il2rgtm1^{Wijl/SzJ} (NSG) mice were dosed subcutaneously with 4×10⁶ CD19 ligand expressing M28 tumor cells. Bulk CD3+ T-cells were co-transduced with the anti-CD19 Hybrid SynNotch CAR with ICDs as indicated in 14B and the BFP reporter as described in FIG. 2. 3×10⁶ transduced CD3+ T cells were dosed to animals via retro-orbital injection 7 days post tumor injection. FIG. 14B show tumor volume assessed via caliper measurements weekly.

FIGS. 15A-15B shows BCMA and ALPPL2 Targeted Hybrid SynNotch CAR Expression and Circuit Induction. T-cells expressing anti-BCMA and anti-ALPPL2 receptors with 41BB costimulatory domains and the BFP reporter were produced as described in FIG. 2 (FIG. 15A). Transduced cells were co-cultured with K562 cells (blue), or antigen positive (either BCMA or ALPPL2) K562 cells (red) for 48 hours (FIG. 15B).

FIG. 16 shows ALPPL2 Targeted Hybrid SynNotch CAR In Vivo Efficacy. NOD.Cg-Prkdc^{scid}Il2rgtm1^{Wijl/SzJ} (NSG) mice were dosed subcutaneously with 4×10⁶ M28 tumor cells as described in FIG. 14. Bulk CD3+ T-cells were co-transduced with anti-ALPPL2 CAR or the anti-ALPPL2 Hybrid SynNotch CAR with ICDs as indicated in figure and the BFP reporter as described in FIG. 2. 3×10⁶ transduced CD3+ T cells were dosed to animals via retro-orbital injection 7 days post tumor injection. Tumor volume was assessed via caliper measurements weekly.

### DETAILED DESCRIPTION OF THE DISCLOSURE

The present disclosure relates generally to a new class of chimeric receptors designed to combine fast time-scale intracellular signal transduction and long time-scale transcription regulation. In particular, some embodiments of the disclosure provides exemplary chimeric receptors (referred to herein as “hybrid SynNotch CARs”) that incorporate (i) costimulatory domains and stimulatory domains of a CAR, for example, the cytoplasmic tail of the CD3zeta chain, and a transcriptional regulator. The architecture of the cytoplasmic tail of these new receptors (costimulatory domain, CD3zeta, transcription factor) can be configured in multiple ways. As described in greater detail below, the present disclosure also identifies hybrid receptor architectures that reliably induce proximal T-cell receptor costimulatory signals and gene regulation in a target cell type, such as, primary human T cells. The new hybrid SynNotch CARs provided herein can simultaneously stimulate (i) fast time-scale (e.g., from seconds to minutes) proximal signaling and (ii) long-time scale transcriptional regulation that usually takes hours to induce to sufficient levels to observe cellular state changes.

As shown in greater detail below in the present disclosure, certain intracellular configurations, but not others, of the Hybrid SynNotch CARs (e.g., with 4-1BB or CD28 costimulatory domains) exhibit antigen independent induction of the inducible transcriptional reporter element. Further, the present disclosure demonstrates that the spatial configuration of the intracellular domains influences receptor behavior in both the presence and absence of ligand. Additionally, the present disclosure exemplifies that, upon engagement with the target antigen, Hybrid SynNotch CARs functionally induce signaling through their intracellular signaling domains (e.g., 4-1BB or CD28, and CD3zeta), leading to expression of activation markers. In some exemplary embodiments, the present disclosures demonstrates that, upon engagement with antigen, the Hybrid SynNotch CARs provided herein functionally induce short term signaling cascades through their intracellular signaling domains (e.g., 4-1BB or CD28, and CD3zeta), leading to proliferation of the T cells. In addition, the present disclosures demonstrates that, the signaling induced by the costimulatory domains and activation domain (e.g., CD3zeta) of the Hybrid SynNotch CARs of the present disclosure differs in type or mechanism, strength, intensity, or length of time to the CARs. In the meantime, the present disclosures demonstrates that the Hybrid SynNotch CAR T cells disclosed herein can kill target cells at similar rates as the CAR T cells. Thus, the present disclosure provides that the Hybrid SynNotch CARs induce T cell activation and cytotoxic programs that are sufficient to cause target cell killing over a period of longer time (such as multiple days). Furthermore, the present disclosure demonstrates that the Hybrid SynNotch CAR T cells are effective in controlling and clearing tumor burden in vivo.

The present disclosure further provides, among others, that modification of the costimulatory domain (e.g., the 4-1BB costimulatory domain) can optimize the Hybrid SynNotch CARs described herein with antigen-independent activity, resulting in improved designs that are capable of both antigen dependent transcriptional circuit induction and T cell signaling.

Although various features of the disclosures may be described in the context of a single embodiment, the features may also be provided separately or in any suitable combination. Conversely, although the disclosures may be described herein in the context of separate embodiments for clarity, the disclosures may also be implemented in a single embodiment. Any published patent applications and any other published references, documents, manuscripts, and scientific literature cited herein are incorporated herein by reference for any purpose. In addition, the materials, methods, and examples are illustrative only and not intended to be limiting.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used herein, refer to the delivery of a composition or formulation by an administration route including, but not limited to, intravenous, intra-arterial, intracerebral, intrathecal, intramuscular, intraperitoneal, subcutaneous, intramuscular, and combinations thereof. The term includes, but is not limited to, administration by a medical professional and self-administration

The term “heterologous” refers to a polypeptide sequence or domain which is not native to a flanking sequence, e.g., wherein the heterologous sequence is not found in nature coupled to the polypeptide sequences occurring at one or both ends.

The term “derived from” as used herein in reference to a protein or polypeptide refers to an origin or source, and may include naturally occurring, recombinant, unpurified or purified polypeptide that is obtained from, is obtained based on a source or original protein or polypeptide. As such, a protein or polypeptide derived from an original protein or polypeptide may include the original protein or polypeptide, in part or in whole, and may be a fragment or variant of the original protein or polypeptide. In some instance, the polypeptide sequence or domain that is derived from a source or origin can be genetically or chemically modified.

The terms “host cell” and “recombinant cell” are used interchangeably herein. It is understood that such terms, as well as “cell”, “cell culture”, “cell line”, refer not only to the particular subject cell or cell line but also to the progeny or potential progeny of such a cell or cell line, without regard to the number of transfers. It should be understood that not all progeny are exactly identical to the parental cell. This is because certain modifications may occur in succeeding generations due to either mutation (e.g., deliberate or inadvertent mutations) or environmental influences, such progeny may not, in fact, be identical to the parent cell, but are still included within the scope of the term as used herein, so long as the progeny retain the same functionality as that of the originally cell or cell line.

The term “operably linked”,” as used herein, denotes a physical or functional linkage between two or more elements, e.g., polypeptide sequences or polynucleotide sequences, which permits them to operate in their intended fashion.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI web site at ncbi.nlm.nih.gov/BLAST. Such sequences are then said to be “substantially identical.” This definition also refers to, or may be applied to, the complement of a test sequence. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, and unless otherwise specified, a “therapeutically effective amount” of an agent is an amount sufficient to provide a therapeutic benefit in the treatment or management of a health condition, such as a disease (e.g., a cancer), or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” is an individual under the care of a physician. Thus, the subject can be a human individual or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, and the like.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

One skilled in the art will understand that the chimeric receptors disclosed herein provide signals having a range of characteristics, from low to high ligand-induced transduction and (independently) low to moderate non-induced signal transduction. This range of activities is a new feature that can be exploited to enhance and tune the actions of engineered cells. Further, as described in greater detail below, a number of the receptor variants disclosed herein exhibit improved expression compared to existing SynNotch receptors.

### Notch Receptors

Notch receptors are large transmembrane proteins that normally communicate signals upon binding to surface-bound ligands expressed on adjacent cells. Notch signals rely on cell-to-cell communication, e.g., communication between two contacting cells, in which one contacting cell is a “receiver” cell and the other contacting cell is a “sender” cell. Notch receptors expressed in a receiver cell recognize their ligands (the delta/serrate/lag, or “DSL” family of proteins) expressed on a sending cell. The engagement of notch and delta on these contacting cells leads to a two-step proteolysis of the notch receptor, which ultimately causes the release of the intracellular portion of the receptor (“ICD”) from the membrane into the cytoplasm. Notch has a matrix metalloprotease cleavage site (denoted “S2”), which, when the receptor is not activated is protected from cleavage by the Notch negative regulatory region (“NRR”). The NRR consists of three LIN-12-Notch repeat (“LNR”) modules and a heterodimerization domain (“HD”). It is believed that this proteolysis is regulated by the force exerted by the sending cell: the DSL ligand pulls on the Notch receptor, which changes the conformation of the NRR and exposes the metalloprotease site. This is cleaved by a constitutively active protease (such as ADAM10), which releases the extracellular binding portion and negative regulatory region of the receptor. Release of the ligand binding portion of the receptor in turn exposes another cleavage site (denoted “S3”), which is cleaved by γ-secretase within the cell membrane: this cleavage releases the nuclear homing ICD from the cell membrane. W.R. Gordon et al., Dev Cell (2015) 33:729-36. This released domain alters receiver cell behavior by regulating transcription. Evolutionary divergence of vertebrates and invertebrates was accompanied by at least two rounds of gene duplication in the Notch lineage: flies possess a single Notch gene, worms two (GLP-1 and LIN-12), and mammals four (NOTCH1-4). Transduction of Notch signals relies on three key events: (i) ligand recognition; (ii) conformational exposure of the ligand-dependent cleavage site; and (iii) assembly of nuclear transcriptional activation complexes.

Canonical Notch signals are transduced by a process called regulated intramembrane proteolysis. Notch receptors are normally maintained in a resting, proteolytically resistant conformation on the cell surface, but ligand binding initiates a proteolytic cascade that releases the intracellular domain of the receptor (ICD) from the membrane. The critical, regulated cleavage step is effected by ADAM metalloproteases and occurs at a site called S2 immediately external to the plasma membrane. This truncated receptor, dubbed NEXT (for Notch extracellular truncation), remains membrane-tethered until it is processed at site S3 by γ-secretase, a multiprotein enzyme complex.

After γ-secretase cleavage, the ICD ultimately enters the nucleus, where it nucleates assembly of a transcriptional activation complex that contains a DNA-binding transcription factor, and a transcriptional coactivator of the Mastermind family. This complex then engages one or more additional coactivator proteins such as p300 to recruit the basal transcription machinery and activate the expression of downstream target genes.

Notch receptors have a modular domain organization. The ectodomains of Notch receptors consist of a series of N-terminal epidermal growth factor (EGF)-like repeats that are responsible for ligand binding. O-linked glycosylation of these EGF repeats, including modification by O-fucose, Fringe, and Rumi glycosyltransferases, also modulates the activity of Notch receptors in response to different ligand subtypes in flies and mammals.

The EGF repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular region, the receptor has a transmembrane segment and an intracellular domain (ICD), which includes a transcriptional regulator.

### Compositions of the Disclosure

The present disclosure provides, among other things, a new class of chimeric receptors designed to combine fast time-scale intracellular signal transduction and long time-scale transcription regulation. In particular, some embodiments of the disclosure provides new hybrid SynNotch receptor architectures that incorporate signaling domains (e.g. co-stimulation, CD3zeta, etc.) that can initiate activation of T cells concomitant with custom transcriptional regulation. In some embodiments, the new receptors provided herein have linear amino acid signaling motif to mediate signaling in T cells added into the cytoplasmic tail of SynNotch receptors. As demonstrated in the Examples and figures, these new receptors can stimulate fast time-scale (e.g., from seconds to a minute) proximal signaling as well as long-time scale transcriptional regulation that takes hours to induce to sufficient levels to observe cellular state changes.

In one aspect, the present disclosure provides chimeric receptor comprising, from N-terminus to C-terminus: a) an extracellular ligand-binding domain having a binding affinity for a selected ligand; b) a linking polypeptide; c) a transmembrane domain comprising one or more ligand-inducible proteolytic cleavage sites; and d) an intracellular domain comprising, in any order: (i) an intracellular signaling domain (SD) comprising (1) at least one costimulatory domain derived from a signaling molecule and (2) an activation domain, and (ii) a transcriptional regulator. In some embodiments, the binding of the selected ligand to the extracellular ligand-binding domain induces cleavage at a ligand-inducible proteolytic cleavage site disposed between the intracellular domain and the linking polypeptide. Further, the binding of the selected ligand to the extracellular ligand-binding domain can also induce proximal signaling cascades through the intracellular SD. In some embodiments, the proximal signaling cascades refer to fast time-scale signaling. For instance, the signaling cascades can be induced in seconds to minutes. Alternatively, the signaling cascades can last for seconds to minutes. In some embodiments, such proximal signaling cascades are induced through T-cell receptor costimulatory signals. In addition, the chimeric receptor provided herein does not comprise a LIN-12-Notch repeat (LNR) and/or a heterodimerization domain (HD) of a Notch receptor. In some embodiments, the linking polypeptide is capable of promoting oligomer formation of the chimeric receptor via intermolecular disulfide bonding.

### Extracellular Domains (ECD) and Ligands

In some embodiments, the ECD of the chimeric receptors (e.g., hybrid SynNotch CARs) disclosed herein has a binding affinity for one or more target ligands. The target ligand can be expressed on the surface of a cell, or is otherwise anchored, immobilized, or restrained so that it can exert a mechanical force on the chimeric receptor. The cell can be a pathogenic cell or a human cell. In some embodiments, the human cell can be a tumor cell. In some embodiments, the human cell can be a terminally differentiated cell. As such, without being bound to any particular theory, binding of the ECD of a chimeric receptor provided herein to a cell-surface ligand does not necessarily remove the target ligand from the target cell surface, but instead enacts a mechanical pulling force on the chimeric receptor. For example, an otherwise soluble ligand may be targeted if it is bound to a surface, or to a molecule in the extracellular matrix. In some embodiments, the target ligand is a cell-surface ligand. Non-limiting examples of suitable ligand types include cell surface receptors; adhesion proteins; carbohydrates, lipids, glycolipids, lipoproteins, and lipopolysaccharides that are surface-bound; integrins; mucins; and lectins. In some embodiments, the ligand is a protein. In some embodiments, the ligand is a carbohydrate.

In some embodiments, the ligand is a cluster of differentiation (CD) marker. In some embodiments, the CD marker is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, and MAGE.

In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment thereof. One skilled in the art upon reading the present disclosure will readily understand that the term “functional fragment thereof” or “functional variant thereof” refers to a molecule having quantitative and/or qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For instance, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety is selected from the group consisting of an antibody, a nanobody, a diabody, a triabody, or a minibody, an F(ab′)2 fragment, an F(ab) fragment, a single chain variable fragment (scFv), and a single domain antibody (sdAb), or a functional fragment thereof. In some embodiments, the antigen-binding moiety includes an scFv.

The antigen-binding moiety can include naturally-occurring amino acid sequences or can be engineered, designed, or modified to provide desired and/or improved properties such as, e.g., binding affinity. Generally, the binding affinity of an antigen-binding moiety, e.g., an antibody, for a target antigen (e.g., CD19 antigen) can be calculated by the Scatchard method described by Frankel et al., Mol. Immunol, 16:101-06, 1979. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. An antibody that “selectively binds” an antigen (such as CD19) is an antigen-binding moiety that does not significantly bind other antigens but binds the antigen with high affinity, e.g., with an equilibrium constant (KD) of 100 nM or less, such as 60 nM or less, for example, 30 nM or less, such as, 15 nM or less, or 10 nM or less, or 5 nM or less, or 1 nM or less, or 500 pM or less, or 400 pM or less, or 300 pM or less, or 200 pM or less, or 100 pM or less.

A skilled artisan can select an ECD based on the desired localization or function of a cell that is genetically modified to express a chimeric receptor or hybrid SynNotch CAR of the present disclosure. For example, a chimeric receptor or hybrid SynNotch CAR with an ECD including an antibody specific for a HER2 antigen can target cells to HER2-expressing breast cancer cells. In some embodiments, the ECD of the disclosed hybrid SynNotch CARs is capable of binding a tumor-associated antigen (TAA) or a tumor-specific antigen (TSA). A skill artisan will understand that TAAs include a molecule, such as e.g., protein, present on tumor cells and on normal cells, or on many normal cells, but at much lower concentration than on tumor cells. In contrast, TSAs generally include a molecule, such as e.g., protein which is present on tumor cells but absent from normal cells.

In some cases, the antigen-binding moiety is specific for an epitope present in an antigen that is expressed by a tumor cell, i.e., a tumor-associated antigen. The tumor-associated antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell, a non-Hodgkin B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a myelogenous leukemia cell, a neuroblastoma cell, a glioma, a glioblastoma, a colorectal cancer cell, etc. It will also be understood that a tumor-associated antigen may also be expressed by a non-cancerous cell. In some embodiments, the antigen-binding domain is specific for an epitope present in a tissue-specific antigen. In some embodiments, the antigen-binding domain is specific for an epitope present in a disease-associated antigen.

Non-limiting examples of suitable target antigens include CD19, B7H3 (CD276), BCMA (CD269), alkaline phosphatase, placental-like 2 (ALPPL2), green fluorescent protein (GFP), enhanced green fluorescent protein (EGFP), signal regulatory protein α (SIRPα), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD 117), MUC1, NCAM, PAP, PDGFR-0, PRSS21, PSCA, PSMA, ROR1, SSEA-4, TAG72, TEM1/CD248, TEM7R, TSHR, VEGFR2, ALPI, citrullinated vimentin, cMet, and Axl.

In some embodiments, the target antigen is selected from CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD 117), MUC1, NCAM, PAP, PDGFR-β, PRSS21, PSCA, PSMA, ROR1, SSEA-4, TAG72, TEM1/CD248, TEM7R, TSHR, VEGFR2, ALPI, citrullinated vimentin, cMet, Axl, GPC2, human epidermal growth factor receptor 2 (Her2/neu), CD276 (B7H3), IL-13Rα1, IL-13Rα2, α-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD123, CD93, CD99, CD 117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, AOC3 (VAP-1), CAM-3001, CCL11 (eotaxin-1), CD125, CD147 (basigin), CD154 (CD40L), CD2, CD20, CD23 (IgE receptor), CD25 (a subunit of the heterodimeric IL-2 receptor), CD3, CD4, CD5, IFN-α, IFN-7, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11α), myostatin, OX-40, scleroscin, SOST, TGFβ1, TNF-α, VEGF-A, pyruvate kinase isoenzyme type M2 (tumor M2-PK), CD20, CD5, CD7, CD3, TRBC1, TRBC2, BCMA, CD38, CD123, CD93, CD34, CD1α, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin.

Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), Ral-B, GPC2, CD276 (B7H3), or IL-13Rα. In some embodiments, the antigen is Her2. In some embodiments, the antigen is ALPPL2. In some embodiments, the antigen is BCMA. In some embodiments, the antigen-binding moiety of the ECD is specific for a reporter protein, such as BFP, GFP, and eGFP. Non-limiting examples of such antigen binding moiety include a LaG17 anti-GFP nanobody. In some embodiments, the antigen-binding moiety of the ECD includes an anti-BCMA fully-humanized VH domain (FHVH). In some embodiments, the antigen is signal regulatory protein α (SIRPα).

Additional antigens suitable for targeting by the chimeric receptors disclosed herein include, but are not limited to GPC2, human epidermal growth factor receptor 2 (Her2/neu), CD276 (B7H3), IL-13Rα1, IL-13Rα2, α-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1.

Additional antigens suitable for targeting by the chimeric receptors disclosed herein include, but are not limited to, those associated with an inflammatory disease such as, AOC3 (VAP-1), CAM-3001, CCL11 (eotaxin-1), CD125, CD147 (basigin), CD154 (CD40L), CD2, CD20, CD23 (IgE receptor), CD25 (a subunit of the heteromeric of IL-2 receptor), CD3, CD4, CD5, IFN-α, IFN-7, IgE, IgE Fc region, IL-1, IL-12, IL-23, IL-13, IL-17, IL-17A, IL-22, IL-4, IL-5, IL-5, IL-6, IL-6 receptor, integrin α4, integrin α407, LFA-1 (CD11a), myostatin, OX-40, scleroscin, SOST, TGFβ1, TNF-α, and VEGF-A.

Further antigens suitable for targeting by the chimeric receptors and hybrid SynNotch CARs disclosed herein include, but are not limited to the pyruvate kinase isoenzyme type M2 (tumor M2-PK), CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin. Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), and Ral-B. In some embodiments, the antigen is GPC2, CD19, Her2/neu, CD276 (B7H3), IL-13Rα1, or IL-13Rα2. In some embodiments, the antigen is Her2. In some embodiments, the antigen is ALPPL2. In some embodiments, the antigen is BCMA. In some embodiments, the antigen-binding moiety of the ECD is specific for a reporter protein, such as GFP and eGFP. Non-limiting examples of such antigen binding moiety include a LaG17 anti-GFP nanobody. In some embodiments, the antigen-binding moiety of the ECD includes an anti-BCMA fully-humanized VH domain (FHVH).

In some embodiments, antigens suitable for targeting by the chimeric receptors and hybrid SynNotch CARs disclosed herein include ligands derived from a pathogen. For example, the antigen can be HER2 produced by HER2-positive breast cancer cells. In some embodiments, the antigen can be CD19 that is expressed on B-cell leukemia. In some embodiments, the antigen can be EGFR that is expressed on glioblastoma multiform (GBM) but much less expressed so on healthy CNS tissue. In some embodiments, the antigen can be CEA that is associated with cancer in adults, for example colon cancer.

In some embodiments, the antigen-binding moiety of the ECD is specific for a cell surface target, where non-limiting examples of cell surface targets include CD19, CD30, Her2, CD22, ENPP3, EGFR, CD20, CD52, CD11α, and α-integrin. In some embodiments, the chimeric receptors and hybrid SynNotch CARs disclosed herein include an extracellular domain having an antigen-binding moiety that binds CD19, CEA, HER2, MUC1, CD20, ALPPL2, BCMA, or EGFR. In some embodiments, the chimeric receptors provided herein (e.g., hybrid SynNotch CARs) include an extracellular domain including an antigen-binding moiety that binds CD19. In some embodiments, the chimeric receptors provided herein (e.g., hybrid SynNotch CARs) include an extracellular domain including an antigen-binding moiety that binds ALPPL2. In some embodiments, the chimeric receptors provided herein (e.g., hybrid SynNotch CARs) include an extracellular domain including an antigen-binding moiety that binds BCMA. In some embodiments, the chimeric receptors provided herein (e.g., hybrid SynNotch CARs) include an extracellular domain including an antigen-binding moiety that binds Her2. In some embodiments, the chimeric receptors and hybrid SynNotch CARs disclosed herein include an extracellular domain including an antigen-binding moiety that binds CD19, ALPPL2, BCMA, or Her2.

In some embodiments, the extracellular domain includes an amino acid sequence having at least 70%, at least 75%, at least 80%, at least 85%, at least 90%, at least 95%, at least 96%, at least 97%, at least 98%, at least 99% sequence identity to a sequence set forth in SEQ ID NOS: 1, 2, 46, and 47. In some embodiments, the extracellular domain includes an amino acid sequence having at least 90% sequence identity to a sequence set forth in SEQ ID NOS: 1, 2, 46, and 47. In some embodiments, the extracellular domain includes an amino acid sequence having at least 95% sequence identity to a sequence set forth in SEQ ID NOS: 1, 2, 46, and 47. In some embodiments, the extracellular domain includes an amino acid sequence having 100% sequence identity to a sequence set forth in SEQ ID NOS: 1, 2, 46, and 47. In some embodiments, the extracellular domain includes an amino acid sequence set forth in SEQ ID NOS: 1, 2, 46, and 47, wherein one, two, three, four, or five of the amino acid residues in any one of the SEQ ID NOS: 1, 2, 46, and 47 is/are substituted by a different amino acid residue.

### Linking Polypeptide

As described above, the chimeric receptors of the disclosure include a linking polypeptide sequence disposed between the extracellular binding domain (ECD) and the transmembrane domain (TMD). Existing “SynNotch” receptors comprise a heterologous extracellular ligand-binding domain, a linking polypeptide having substantial sequence identity with a Notch receptor JMD including the NRR, a TMD, and an ICD. In contrast, the chimeric receptors and hybrid SynNotch CARs comprise a heterologous extracellular ligand-binding domain, a linking polypeptide having substantial sequence identity with a Notch receptor JMD but lacking the NRR (the LIN-12-Notch repeat (LNR) modules, and the heterodimerization domain), a TMD, and an ICD. Stated differently, in hybrid SynNotch CARs, the linking polypeptide replaces the negative regulatory region (NRR) and heterodimerization (HD) domain of the native Notch. Three to 50 amino acid residues (e.g., 3, 4, 5, 6, 7, 8, 9, 10, 11, 12, 13, 14, 15, 16, 17, 18, 19, 20, etc. amino acid residues) can be used as a polypeptide linker. In some embodiments, the length and amino acid composition of the linker polypeptide sequence can be optimized to vary the orientation and/or proximity of the ECD and the TMD relative to one another to achieve a desired activity of the chimeric receptor of the disclosure. All of these sequences can be used as a linking polypeptide for the chimeric receptors of the present disclosure.

In some embodiments, the linking polypeptide encompassed by the present disclosure can include a polypeptide capable of promoting oligomer formation of the chimeric receptor via intermolecular disulfide bonding, for example, a hinge linker. In some embodiments, hinge linkers of the disclosure include an oligomerization domain (e.g., a hinge domain) containing one or more polypeptide motifs that promote oligomer formation of the chimeric receptors via intermolecular disulfide bonding. In these instances, within the chimeric receptors disclosed herein, the hinge domain generally includes a flexible polypeptide connector region disposed between the ECD and the TMD. Thus, the hinge domain provides flexibility between the ECD and TMD and also provides sites for intermolecular disulfide bonding between two or more chimeric receptor monomers to form an oligomeric complex. In some embodiments, the hinge domain includes motifs that promote dimer formation of the chimeric receptors disclosed herein. In some embodiments, the hinge domain includes motifs that promote trimer formation of the chimeric receptors disclosed herein (e.g., a hinge domain derived from OX40). Hinge polypeptide sequences suitable for the compositions and methods of the disclosure can be naturally-occurring hinge polypeptide sequences (e.g., those from naturally-occurring immunoglobulins) or can be engineered, designed, or modified so as to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof. In some embodiments, the hinge polypeptide sequence contains one or more CXXC motifs. In some embodiments, the hinge polypeptide sequence contains one or more CPPC motifs. Additional information in this regard can be found in, for example, a recent review by G. Vidarsson et al., Frontiers Immunol (2014) 5:520 (doi: 10.3389/fimmu.2014.00520), which is hereby incorporated by reference in its entirety.

Hinge polypeptide sequences can also be derived from a CD8a hinge domain, a CD28 hinge domain, a CD152 hinge domain, a PD-1 hinge domain, a CTLA4 hinge domain, an OX40 hinge domain, and functional variants thereof. In some embodiments, the hinge domain includes a hinge polypeptide sequence derived from a CD8a hinge domain or a functional variant thereof. In some embodiments, the hinge domain includes a hinge polypeptide sequence derived from a CD28 hinge domain or a functional variant thereof. In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

The hinge linker can include about 5 to about 60 amino acids from or overlapping with the selected hinge domain, for example at least about 5, at least about 6, at least about 7, at least about 8, at least about 9, at least about 10, at least about 12, at least about 15, at least about 17, at least about 20, at least about 22, at least about 24, at least about 26, at least about 28, at least about 30, at least about 35, at least about 40, at least about 45, at least about 50, at least about 55, or, at least about 60 amino acids. In embodiments of the invention, the Hinge linker has no more than about 60 amino acids, less than about 55, less than about 50, less than about 45, less than about 40, less than about 35, less than about 32, less than about 30, less than about 29, less than about 28, less than about 27, less than about 26, less than about 25, less than about 24, less than about 23, less than about 22, less than about 21, less than about 20, less than about 18, less than about 16, less than about 14, less than about 12, or less than about 10 amino acids.

In some embodiments, the linking polypeptide sequence includes a sequence having at least 80% sequence identity, such as, at least 80%, at least 85%, at least 90%, at least 95%, at least 96%, at least 97%, at least 98%, or 99% sequence identity to a sequence set forth in SEQ ID NO: 3. In some embodiments, the linking polypeptide sequence includes an amino acid sequence having at least 90% sequence identity to a sequence set forth in SEQ ID NO: 3. In some embodiments, the linking polypeptide sequence includes an amino acid sequence having at least 95% sequence identity to a sequence set forth in SEQ ID NO: 3. In some embodiments, the linking polypeptide sequence includes an amino acid sequence having at least 99% sequence identity to a sequence set forth in SEQ ID NO: 3. In some embodiments, the linking polypeptide sequence includes an amino acid sequence identical to a sequence set forth in SEQ ID NO: 3. In some embodiments, the linking polypeptide sequence includes an amino acid sequence set forth in SEQ ID NO: 3, wherein one, two, three, four, or five of the amino acid residues in any one of the SEQ ID NO: 3 is/are substituted by a different amino acid residue.

### Transmembrane Domains (TMD)

As described above, the chimeric receptors of the disclosure include a TMD comprising one or more ligand-inducible proteolytic cleavage sites.

Examples of proteolytic cleavage sites in a Notch receptor (e.g., S2 or S3) are as described above. Additional proteolytic cleavage sites suitable for the compositions and methods disclosed herein include, but are not limited to, a metalloproteinase cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue such as Leu, Ile, Val, Phe, Trp, Tyr, Val, Met, and Pro), e.g., Pro-X-X-Hy-(Ser/Thr), e.g., Pro-Leu/Gln-Gly-Met-Thr-Ser or Pro-Leu/Gln-Gly-Met-Thr. Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., Glu-Asn-Leu-Tyr-Thr-Gln-Ser, where the protease cleaves between the glutamine and the serine. Another example of a protease cleavage site that can be included in a proteolytically cleavable linker is an enterokinase cleavage site, e.g., Asp-Asp-Asp-Asp-Lys, where cleavage occurs after the lysine residue. Another example of a protease cleavage site that can be included in a proteolytically cleavable linker is a thrombin cleavage site, e.g., Leu-Val-Pro-Arg. Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr virus protease, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrix metalloproteinase (MT-MMP), stromelysin 3 (or MMP-11), thermolysin, fibroblast collagenase and stromelysin-1, matrix metalloproteinase 13 (collagenase-3), tissue-type plasminogen activator (tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the receptor is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

Generally, the TMD suitable for the chimeric receptors disclosed herein can be any transmembrane domain of a Type 1 transmembrane receptor including at least one γ-secretase cleavage site. Detailed description of the structure and function of the γ-secretase complex as well as its substrate proteins, including amyloid precursor protein (APP) and Notch, can, for example, be found in a recent review by Zhang et al., Frontiers Cell Neurosci (2014). Non-limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one γ-secretase cleavage site. Additional TMDs suitable for the compositions and methods described herein include, but are not limited to, transmembrane domains from Type 1 transmembrane receptors IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric receptors of the disclosure is a TMD derived from the TMD of a member of the calsyntenin family, such as, alcadein alpha and alcadein gamma. In some embodiments, the TMD of the chimeric receptors of the disclosure is a TMD known for Notch receptors. In some embodiments, the TMD of the chimeric receptors of the disclosure is a TMD derived from a different Notch receptor. For example, in a Mini Notch based on human Notch1, the Notch1 TMD can be substituted with a Notch2 TMD, Notch3 TMD, Notch4 TMD, or a Notch TMD from a non-human animal such as Danio rerio, Drosophila melanogaster, Xenopus laevis, or Gallus gallus.

In some embodiments, the amino acid substitution(s) within the TMD includes one or more substitutions within a “GV” motif of the TMD. In some embodiments, at least one of such substitution(s) comprises a substitution to alanine. For example, one, two, three, four, five, or more of the amino acid residues of the sequence FMYVAAAAFVLLFFVGCGVLL (SEQ ID NO: 4) may be substituted by a different amino acid residue. In some embodiments, the amino acid residue at position 18 and/or 19 of the “GV” motif within SEQ ID NO: 4 is substituted by a different amino acid residue. In some embodiments, the glycine residue at position 18 of SEQ ID NO: 4 is substituted by a different amino acid residue. In some embodiments, the valine residue at position 19 of SEQ ID NO: 4 is substituted by a different amino acid residue. In some embodiments, the transmembrane domain comprises an amino acid sequence having a sequence corresponding to SEQ ID NO: 4 with a mutation at the position corresponding to position 18 of SEQ ID NO: 4, such as G18A mutations. In some embodiments, the transmembrane domain comprises an amino acid sequence having a sequence corresponding to SEQ ID NO: 4 with a mutation at the position corresponding to position 19 of SEQ ID NO: 4, such as V19A mutations. The TMD can be derived from but longer or shorter than SEQ ID NO: 4. For instance, the TMD can be one, two, three, four, or more amino acids longer or shorter than SEQ ID NO: 4. In some embodiments, the TMD includes a sequence having at least 80% sequence identity, such as, at least 80%, at least 85%, at least 90%, at least 95%, at least 96%, at least 97%, at least 98%, or 99% sequence identity to SEQ ID NO: 4.

### Stop-Transfer Sequence (STS)

The chimeric receptors of the disclosure include an STS which comprises a charged, hydrophilic domain located between the TMD and the ICD. Without being bound to any particular theory, this domain disposed between the TMD and the ICD prevents the ICD from entering the plasma membrane. In some embodiments, a single-chain peptide comprising about 1 to about 40 amino acid residues (e.g., 1, 2, 3, 4, 5, 6, 7, 8, 9, 10, 11, 12, 13, 14, 15, 16, 17, 18, 19, 20, or more amino acid residues) in which most of the residues have charged side chains under physiological conditions can be used as a STS. In short STS embodiments (e.g., less than about 6 amino acids), about 5 or 6 of the amino acids will have charged side chains. In some embodiments, the STS includes about 1 to 15, about 5 to 20, about 8 to 25, about 10 to 30, about 12 to 35, about 14 to 40, about 5 to 40, about 10 to 35, about 15 to 30, about 20 to 25, about 20 to 40, about 10 to 30, about 4 to 20, or about 5 to 25 amino acid residues. In some embodiments, the STS includes about 4 to 10, about 5 to 12, about 6 to 14, about 7 to 18, about 8 to 20, about 9 to 22, about 10 to 24, or about 11 to 26 amino acid residues. In some embodiments, the STS includes about 4 to 10 residues, such as, 4, 5, 6, 7, 8, 9, or 10 amino acid residues.

In some embodiments, the STS includes a sequence having at least about 80% sequence identity, such as, at least 80%, at least 85%, at least 90%, at least 95%, at least 96%, at least 97%, at least 98%, or 99% sequence identity to the STS domain of a Type 1 receptor. In some embodiments, the STS includes an amino acid sequence having at least 90% sequence identity to the STS domain of a Type 1 receptor. In some embodiments, the STS includes a sequence having at least 70% sequence identity, such as, at least 70%, at least 75%, at least 80%, at least 85%, at least 90%, at least 95%, at least 96%, at least 97%, at least 98%, or 99% sequence identity to a STS sequence from Notch1, Notch2, Notch3, Notch4, CSF1R, CXCL16, DAG1, GHR, PTPRF, AGER, KL, NRG1, LRP1B, Jag2, EPCAM, KCNE3, CDH2, NRG2, PTPRK, BTC, EPHA3, IL1R2, or PTPRM. In some embodiments, the STS includes a sequence comprising only Lys (K) or Arg (R) in the first 4 residues. In some embodiments, the STS includes one, two, three, four, five, or more basic residues. In some embodiments, the STS includes five, four, three, two, one, or zero aromatic residues or residues with hydrophobic and/or bulky side chains.

In some embodiments, the STS includes a sequence having at least 80% sequence identity, such as, at least 80%, at least 85%, at least 90%, at least 95%, at least 96%, at least 97%, at least 98%, or 99% sequence identity to SKRKRKH (SEQ ID NO: 5). In some embodiments, the STS includes an amino acid sequence having at least 90% sequence identity to SEQ ID NO: 5. The STS can be derived from but longer or shorter than SEQ ID NO: 5. For instance, the STS can be one, two, three, four, or more amino acids longer or shorter than SEQ ID NO:5. In some embodiments, the STS includes a sequence having at least 80% sequence identity, such as, at least 80%, at least 85%, at least 90%, at least 95%, at least 96%, at least 97%, at least 98%, or 99% sequence identity to SEQ ID NO:5. In some embodiments, the STS includes an amino acid sequence having at least 95% sequence identity to SEQ ID NO: 5. In some embodiments, the STS includes an amino acid sequence having at least 100% sequence identity to SEQ ID NO: 5. In some embodiments, the STS includes the amino acid sequence of SEQ ID NO: 5, wherein one, two, three, four, or five of the amino acid residues in SEQ ID NO: 5 is/are substituted by a different amino acid residue.

### Intracellular Domain (ICD)

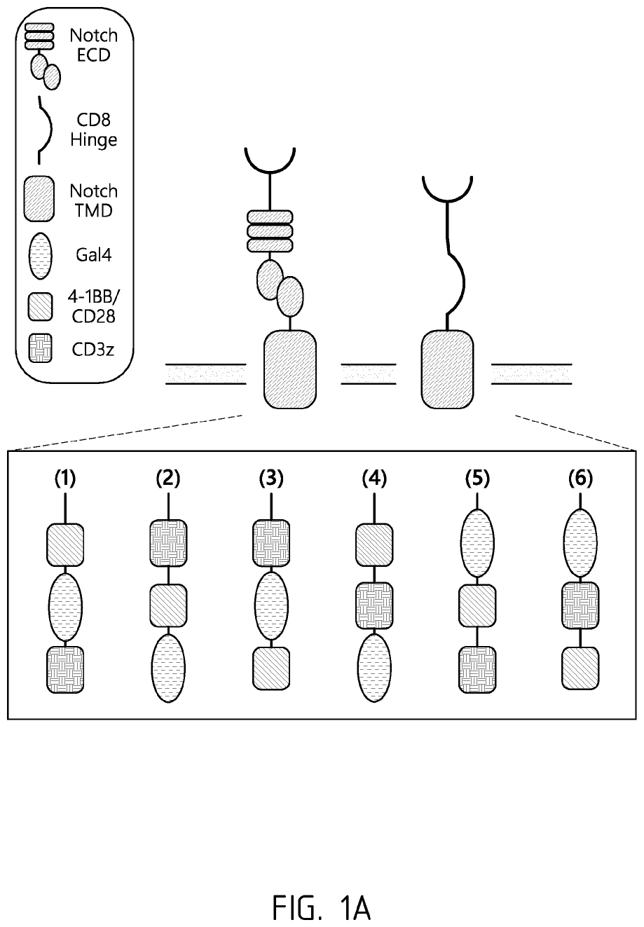

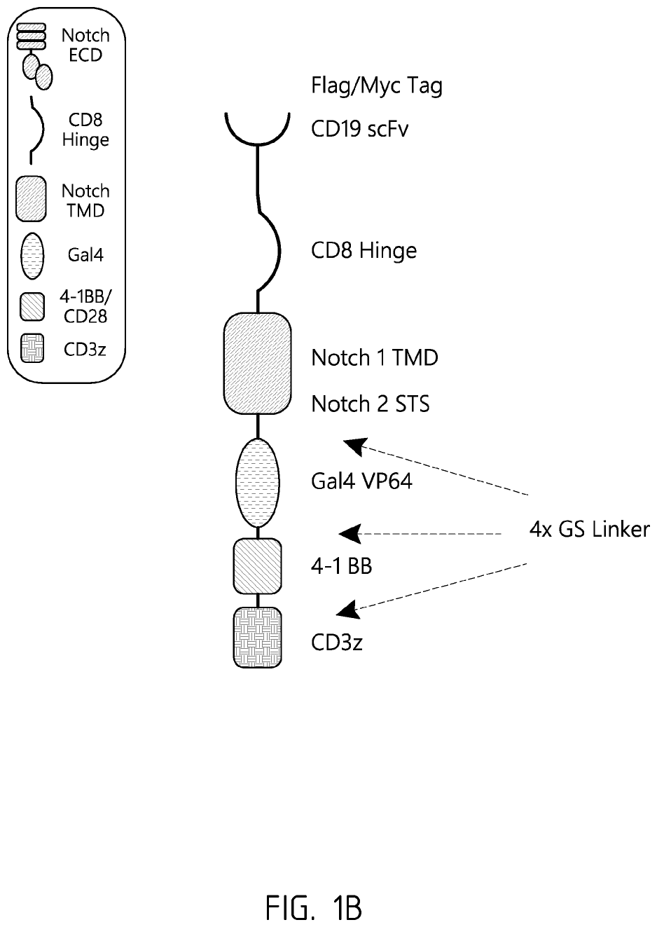

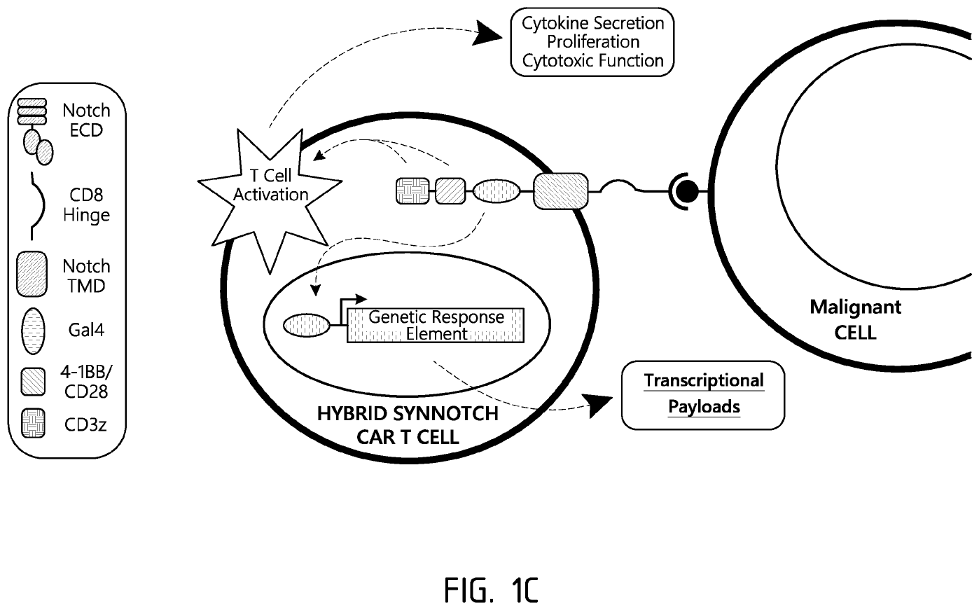

The chimeric receptors of the disclosure includes an intracellular domain (ICD) comprising, in any order: (i) an intracellular signaling domain (SD) comprising at least one costimulatory domain derived from a signaling molecule and an activation domain, and (ii) a transcriptional regulator. In other words, the ICD of the chimeric receptors of the disclosure can have at least three distinct domains, as depicted in FIGS. 1A-1B. The three distinct domains can be arranged in specific orders, and can be operably linked to one another via one or more linkers. In some embodiments, as shown in FIG. 1B, the three distinct domains are linked via (GS)n linkers. The n can be any number selected from 1 to 100. For example, the n can be 2, 3, 4, 5, 6, 7, 8, 9, or 10. In other embodiments, the n can be 11, 12, 13, 14, 15, 16, 17, 18, 19, or 20. An exemplary GS linker can have 3 GS repeats and the sequence of GSGSGSGS (SEQ ID NO: 6). One skilled in the art would know how to modify the linker to suit specific uses.

In some embodiments, the intracellular domain of the chimeric receptors of the disclosure further comprises an intracellular signaling domain. The intracellular signaling domain can have at least two distinct domains: at least one costimulatory domain and an activation domain.

In some embodiments, the costimulatory domain comprises a sequence that is derived from a signaling molecule. The signaling molecule can be a protein selected from a class 1 or a class 3 human membrane protein. In some embodiments, the signaling molecule is selected from CD28, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, 4-1BB, OX40, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-P1A, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC, and derivatives, mutants, variants, fragments and combinations thereof. In other embodiments, the signaling molecule is selected from the list consisting of OX40, ICOS, 4-1BB, CTLA4, CD28, CD30, CD2, CD27, and CD226, and derivatives, mutants, variants, fragments and combinations thereof. In some embodiments, the signaling molecule is selected from the group consisting of 4-1BB, BAFF-R, BCMA, BTLA, CD2, CD200R, CD244, CD28, CD300a, CD300f, CD40, CD7, CD72, CD96, CRACC, CRTAM, CTLA4, CXADR, DC-SIGN, GITR, HAVCR2, ICOS, ILT2, ILT3, ILT4, KIR2DL1, KIR3DL1, KLRG1, LAG3, LAIR1, NKG2D, NKR-P1A, NTB-A, PD1, Siglec-3, TACI, TIGIT, TLT-1, and TNR8 (CD30), and derivatives, mutants, variants, fragments and combinations thereof. In other embodiments, the signaling molecule is CD28 or 4-1BB. In one exemplary embodiment, the costimulatory domain comprises a sequence that is derived from CD28. In another exemplary embodiment, the costimulatory domain comprises a sequence that is derived from 4-1BB. In another embodiment, the costimulatory domain comprises one of the CD28 signaling motifs appended to the C terminus of a trunc41BB costimulatory domain.

In some embodiments, the activation domain includes one or more conserved amino acid motifs that serve as substrates for phosphorylation such as, for example, immunoreceptor tyrosine-based activation motifs (ITAMs). In some embodiments, the activation domain includes at least 1, at least 2, at least 3, at least 4, or at least 5 specific tyrosine-based motifs selected from ITAM motifs, an ITIM motifs, or related intracellular motifs that serve as a substrate for phosphorylation. In some embodiments of the disclosure, the activation domain of the intracellular signaling domain includes at least 1, at least 2, at least 3, at least 4, or at least 5 ITAMs. Generally, any activation domain including an ITAM can be suitably used for the construction of the chimeric receptor s as described herein. An ITAM generally includes a conserved protein motif that is often present in the tail portion of signaling molecules expressed in many immune cells. The motif may include two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways

In some embodiments, the activation domain is derived from CD3ζ, CD3σ, CD3/, and CD3ε. For instance, in some embodiments, the ITAMs are derived from CD3ζ, CD3σ, CD3/, and CD3ε. In one exemplary embodiment, the ITAM is derived from CD3ζ. In certain embodiments, the ITAM comprises a sequence that is at least about 80, 85, 90, 95, 96, 97, 98, 99, or 100% identical to a CD3ζ ITAM. In some embodiments, the activation domain comprises at least 1, at least 2, at least 3, at least 4, or at least 5 ITAMs independently selected from the ITAMs derived from CD3ζ, FcRγ, and combinations thereof. In some embodiments, the activation domain comprises a CD3ζ ITAM.

In some embodiments, the intracellular domain of the chimeric receptors of the disclosure further comprises a transcriptional regulator. The transcriptional regulator is a biochemical element that acts to activate or repress the transcription of a promoter-driven DNA sequence. Transcriptional regulators suitable for the compositions and methods of the disclosure can be naturally-occurring transcriptional regulators or can be engineered, designed, or modified so as to provide desired and/or improved properties, e.g., modulating transcription. In some embodiments, the transcriptional regulator directly regulates expression of one or more genes involved in differentiation of the cell. In some embodiments, the transcriptional regulator indirectly modulates expression of one or more genes involved in differentiation of the cell by modulating the expression of a second transcription factor which in turn modulates expression of one or more genes involved in differentiation of the cell. It will be understood by a skilled artisan that a transcriptional regulator can be a transcriptional activator or a transcriptional repressor. In some embodiments, the transcriptional regulator is a transcriptional repressor. In some embodiments, the transcriptional regulator is a transcriptional activator. In some embodiments, the transcriptional regulator can further include a nuclear localization signal. In some embodiments, the transcriptional regulator comprises a nuclear localization sequence derived from Gal4, tetR, ZFHD1, or HAP1. In other embodiments, the transcriptional regulator comprises a transcriptional regulator sequence derived from VP64, VP65, KRAB, or VP16. In certain embodiments, the transcriptional regulator is selected from Gal4-VP16, Gal4-VP64, tetR-VP64, ZFHD1-VP64, Gal4-KRAB, and HAP1-VP16. In some embodiments, the transcriptional regulator is Gal4-VP64.

In some embodiments, the ICD includes a sequence having at least 80% sequence identity, such as, at least 80%, at least 85%, at least 90%, at least 95%, at least 96%, at least 97%, at least 98%, or 99% sequence identity to one or more of SEQ ID NOs: 7-14, 33, 49-53, and 59-62. In some embodiments, the ICD includes an amino acid sequence having at least 90% sequence identity to one or more of SEQ ID NOs: 7-14, 33, 49-53, and 59-62. In some embodiments, the ICD includes an amino acid sequence having at least 95% sequence identity to one or more of SEQ ID NOs: 7-14, 33, 49-53, and 59-62. In some embodiments, the ICD includes an amino acid sequence having at least 100% sequence identity to one or more of SEQ ID NOs: 7-14, 33, 49-53, and 59-62. In some embodiments, the ICD includes an amino acid sequence of one or more of SEQ ID NOs: 7-14, 33, 49-53, and 59-62, wherein one, two, three, four, or five of the amino acid residues in one or more of SEQ ID NOs: 7-14, 33, 49-53, and 59-62 is/are substituted by a different amino acid residue.

### Additional Domains

In some embodiments, the chimeric receptors provided herein can further include an additional region or domain. For example, in some embodiments, the extracellular domains located N-terminally to the TMD can include a membrane localization signal such as a CD8A signal. In other embodiments, the chimeric receptors can include a detectable label, such as a myc tag or His tag, and the like. In additional embodiments, the chimeric receptors provided herein can also include a tumor-specific cleavage site, or a disease-specific cleavage site. In further embodiments, the chimeric receptors provided herein can include a combination of these additional regions.

In some embodiments, the chimeric receptors of the disclosure include: (a) a linking polypeptide including an amino acid sequence having at least 80% sequence identity to SEQ ID NO: 3; (b) a transmembrane domain including an amino acid sequence having at least 80% sequence identity to SEQ ID NO: 4; and (c) a stop transfer sequence domain including an amino acid sequence having at least 80% sequence identity to SEQ ID NO: 5.

In some embodiments, the chimeric receptors of the disclosure include: (a) an extracellular ligand-binding domain having at least 80% sequence identity to any one of SEQ ID NOS: 1, 2, 45, and 46; (b) a linking polypeptide including an amino acid sequence having at least 80% sequence identity to SEQ ID NO: 3; (c) a transmembrane domain including an amino acid sequence having at least 80% sequence identity to SEQ ID NO: 4; (d) a stop transfer sequence domain including an amino acid sequence having at least 80% sequence identity to SEQ ID NO: 5; and (e) an intracellular domain comprising including one or more amino acid sequences having at least 80% sequence identity to one or more of SEQ ID NOs: 7-14, 33, 49-53, and 59-62.

In some embodiments, the chimeric receptors of the disclosure include: (a) an extracellular ligand-binding domain having at least 80% sequence identity to any one of SEQ ID NOS: 1, 2, 45, and 46; (b) a linking polypeptide including an amino acid sequence having at least 80% sequence identity to SEQ ID NO: 3; (c) a transmembrane domain including an amino acid sequence having at least 80% sequence identity to SEQ ID NO: 4; (d) a stop transfer sequence domain including an amino acid sequence having at least 80% sequence identity to SEQ ID NO: 5; and (e) an intracellular domain comprising including three amino acid sequences, each having at least 80% sequence identity to any one of SEQ ID NOs: 7-14, 33, 49-53, and 59-62, linked by a GS linker.

In some exemplary embodiments, the chimeric receptors of the disclosure includes: (a) an extracellular ligand-binding domain having a sequence set forth in SEQ ID NO: 1, 2, 45, and 46; (b) a linking polypeptide including an amino acid sequence having a sequence set forth in SEQ ID NO: 3; (c) a transmembrane domain including an amino acid sequence having a sequence set forth in SEQ ID NO: 4; (d) a stop transfer sequence domain including an amino acid sequence having a sequence set forth in SEQ ID NO: 5; and (e) an intracellular domain comprising including three amino acid sequences, each having a sequence set forth in SEQ ID NOs: 7-14, 33, 49-53, and 59-62, linked by a GS linker.

In some embodiments, the chimeric receptors of the disclosure include: (a) an extracellular ligand-binding domain having at least 80% sequence identity to any one of SEQ ID NOS: 1, 2, 45, and 46; (b) a linking polypeptide including an amino acid sequence having at least 80% sequence identity to SEQ ID NO: 3; (c) a transmembrane domain including an amino acid sequence having at least 80% sequence identity to SEQ ID NO: 4; and (d) an intracellular domain comprising including one or more amino acid sequences having at least 80% sequence identity to one or more of SEQ ID NOs: 7-14, 33, 49-53, and 59-62.

In some embodiments, the chimeric receptor of the disclosure includes an amino acid sequence having at least about 80%, 90%, 95%, 96%, 97, 98%, 99%, or 100% sequence identity to a chimeric receptor disclosed herein. In some embodiments, provided herein are chimeric receptors including an amino acid sequence having at least about 80%, 90%, 95%, 96%, 97, 98%, 99%, or 100% sequence identity to any one of SEQ ID NOs: 15-31, 32-44, 47-48, 54-58, and 63-68.

### Nucleic Acid Molecules

In another aspect, provided herein are various nucleic acid molecules including nucleotide sequences encoding the chimeric receptors and hybrid SynNotch receptors of the disclosure, including expression cassettes, and expression vectors containing these nucleic acid molecules operably linked to heterologous nucleic acid sequences such as, for example, regulatory sequences which facilitate in vivo expression of the receptor in a host cell.

Nucleic acid molecules of the present disclosure can be of any length, including for example, between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, provided herein is a nucleic acid molecule including a nucleotide sequence encoding a chimeric receptor or hybrid SynNotch receptor including, from N-terminus to C-terminus: (a) an extracellular ligand-binding domain having a binding affinity for a selected ligand; (b) a linking sequence; (c) a transmembrane domain including one or more ligand-inducible proteolytic cleavage sites; and (d) an intracellular domain including (i) an intracellular signaling domain (SD) comprising at least one costimulatory domain derived from a signaling molecule and an activation domain, and (ii) a transcriptional regulator, wherein binding of the selected ligand to the extracellular ligand-binding domain induces cleavage at a ligand-inducible proteolytic cleavage site disposed between the transcriptional regulator and the hinge domain.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the chimeric receptor as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, provided herein are nucleic acid molecules encoding a polypeptide with an amino acid sequence having at least about 80%, 90%, 95%, 96%, 97, 98%, 99%, or 100% sequence identity to a chimeric receptor disclosed herein. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOS: 1-68. In some embodiments, the nucleic acid molecules encode a polypeptide with an amino acid sequence having at least about 80%, 90%, 95%, 96%, 97, 98%, 99%, or 100% sequence identity to any one of SEQ ID NOS: 15-32, 34-44, 47-48, 54-58, and 63-68. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOS: 1 and 2. In some embodiments, provided herein are nucleic acid molecules encoding a polypeptide with an amino acid sequence having at least about 80%, 90%, 95%, 96%, 97, 98%, 99%, or 100% sequence identity to SEQ ID NO: 3. In some embodiments, provided herein are nucleic acid molecules encoding a polypeptide with an amino acid sequence having at least about 80%, 90%, 95%, 96%, 97, 98%, 99%, or 100% sequence identity to SEQ ID NO: 4. In some embodiments, provided herein are nucleic acid molecules encoding a polypeptide with an amino acid sequence having at least about 80%, 90%, 95%, 96%, 97, 98%, 99%, or 100% sequence identity to SEQ ID NO: 5. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOs: 7-14, 33, 49-53, and 59-62.

The nucleic acid sequences encoding the chimeric receptors can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the chimeric receptors disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any chimeric receptor or hybrid SynNotch receptor disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic host, such as a mammalian cells (e.g., COS cells, NIH 3T3 cells, or HeLa cells). These cells are available from many sources, including the American Type Culture Collection (Manassas, VA). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

### Recombinant Cells and Cell Cultures

The nucleic acid of the present disclosure can be introduced into a host cell, such as, for example, a human T lymphocyte, to produce a recombinant or engineered cell containing the nucleic acid molecule. Accordingly, some embodiments of the disclosure relate to methods for making a recombinant or engineered cell, including (a) providing a cell capable of protein expression and (b) contacting the provided cell with a recombinant nucleic acid of the disclosure.

Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell (TH), a cytotoxic T cell (TCTL), or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+ T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+ T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from a subject. In some embodiments, the subject is a human patient.

In some embodiments, the recombinant cell further includes a first and a second nucleic acid molecule as disclosed herein, wherein the first nucleic acid molecule and the second nucleic acid molecule do not have the same sequence. In some embodiments, the recombinant cell further includes a first and a second chimeric receptor or hybrid SynNotch receptor as disclosed herein, wherein the first chimeric receptor or hybrid SynNotch receptor and the second chimeric receptor or hybrid SynNotch receptor do not have the same sequence. In some embodiments, the first chimeric receptor or hybrid SynNotch receptor modulates the expression and/or activity of the second chimeric receptor or hybrid SynNotch receptor.

In some embodiments, the recombinant cell further includes an expression cassette encoding a protein of interest operably linked to a promoter, wherein expression of the protein of interest is modulated by the chimeric receptor transcriptional regulator. Any suitable promoter can be used in connection with the present disclosure. In some embodiments, the promoter comprises a yeast GAL4 promoter. In some embodiments, the protein of interest is heterologous to the recombinant cell. A heterologous protein is one that is not normally found in the cell, e.g., not normally produced by the cell. In principle, there are no particular limitations with regard to suitable proteins whose expression can be modulated by the chimeric receptor transcriptional regulator. Exemplary types of proteins suitable for use with the compositions and methods disclosed herein include cytokines, cytotoxins, chemokines, immunomodulators, pro-apoptotic factors, anti-apoptotic factors, hormones, differentiation factors, dedifferentiation factors, immune cell receptors, or reporters. In some embodiments, the immune cell receptor is a T-cell receptor (TCR). In some embodiments, the immune cell receptor is a chimeric antigen receptor (CAR). In some embodiments, the expression cassette encoding the protein of interest is incorporated into the same nucleic acid molecule that encodes the chimeric receptor of the disclosure. In some embodiments, the expression cassette encoding the protein of interest is incorporated into a second expression vector that is separate from the nucleic acid molecule encoding the chimeric receptor of the disclosure.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the nucleic acids, and recombinant cells of the disclosure can be incorporated into compositions, including pharmaceutical compositions. Such compositions generally include the nucleic acids, and/or recombinant cells, and a pharmaceutically acceptable excipient, e.g., carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the chimeric receptors and Notch receptors of the disclosure can also be administered by transfection or infection using methods known in the art, including but not limited to the methods described in McCaffrey et al. (Nature 418:6893, 2002), Xia et al. (Nature Biotechnol. 20:1006-10, 2002), or Putnam (Am. J. Health Syst. Pharm. 53:151-60, 1996, erratum at Am. J. Health Syst. Pharm. 53:325, 1996).

### Methods of the Disclosure

Administration of any one of the therapeutic compositions described herein, e.g., nucleic acids, recombinant cells, and pharmaceutical compositions, can be used to treat patients for relevant health conditions or diseases, such as cancers and chronic infections. In some embodiments, the nucleic acids, recombinant cells, and pharmaceutical compositions described herein can be incorporated into therapeutic agents for use in methods of treating an individual who has, who is suspected of having, or who may be at high risk for developing one or more autoimmune disorders or diseases associated with checkpoint inhibition. Exemplary autoimmune disorders and diseases can include, without limitation, celiac disease, type 1 diabetes, Graves' disease, inflammatory bowel disease, multiple sclerosis, psoriasis, rheumatoid arthritis, and systemic lupus erythematosus.

Accordingly, in one aspect, some embodiments of the disclosure relate to methods for inhibiting an activity of a target cell in an individual, the methods include administering to the individual a first therapy including one or more of nucleic acids, recombinant cells, and pharmaceutical compositions as disclosed herein, wherein the first therapy inhibits the target cell. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cells disclosed herein, wherein the recombinant cells inhibit an activity of the target cells in the individual. Generally, the target cells of the disclosed methods can be any cell type in an individual and can be, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell.

In another aspect, some embodiments of the disclosure relate to methods for the treatment of a health condition (e.g., disease) in an individual in need thereof, the methods include administering to the individual a first therapy including one or more of the recombinant cells including a chimeric receptor as disclosed herein, and/or pharmaceutical compositions as disclosed herein, wherein the first therapy treats the health condition in the individual. In some embodiments, the methods include administering to the individual a first therapy including an effective number of the recombinant cells as disclosed herein, wherein the recombinant cells treat the health condition.

In another aspect, some embodiments of the disclosure relate to methods for assisting in the treatment of a health condition (e.g., disease) in an individual in need thereof, the methods including administering to the individual a first therapy including one or more of chimeric receptor s, Hinge-Notch receptors, nucleic acids, recombinant cells, and pharmaceutical compositions as disclosed herein, and a second therapy, wherein the first and second therapies together treat the disease in the individual. In some embodiments, the methods include administering to the individual a first therapy including an effective number of the recombinant cells as disclosed herein, wherein the recombinant cells treat the health condition.

### Administration of Recombinant Cells to an Individual

In some embodiments, the methods of the disclosure involve administering an effective amount of the recombinants cells of the disclosure to an individual in need of such treatment. This administering step can be accomplished using any method of implantation delivery in the art. For example, the recombinant cells of the disclosure can be infused directly in the individual's bloodstream or otherwise administered to the individual.

In some embodiments, the methods disclosed herein include administering, which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even the lifetime of the individual, i.e., long-term engraftment.

When provided prophylactically, the recombinant cells described herein can be administered to an individual in advance of any symptom of a disease or condition to be treated. Accordingly, in some embodiments the prophylactic administration of a recombinant cell population prevents the occurrence of symptoms of the disease or condition.

When provided therapeutically in some embodiments, recombinant cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source. In some embodiments, the recombinant cells are expanded in culture prior to administration to an individual in need thereof.

In some embodiments, the delivery of a recombinant cell composition (e.g., a composition including a plurality of recombinant cells according to any of the cells described herein) into an individual by a method or route results in at least partial localization of the cell composition at a desired site. A composition including recombinant cells can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a preferred mode of administration.

In some embodiments, the recombinant cells are administered systemically, e.g., via infusion or injection. For example, a population of recombinant cells are administered other than directly into a target site, tissue, or organ, such that it enters, the individual's circulatory system and, thus, is subject to metabolism and other similar biological processes.

The efficacy of a treatment including any of the compositions provided herein for the treatment of a disease or condition can be determined by a skilled clinician. However, one skilled in the art will appreciate that a treatment is considered effective if any one or all of the signs or symptoms or markers of disease are improved or ameliorated. Efficacy can also be measured by failure of an individual to worsen as assessed by decreased hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular beneficial effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments of the disclosed methods, the individual is a mammal. In some embodiments, the mammal is a human. In some embodiments, the individual has or is suspected of having a disease associated with inhibition of cell signaling mediated by a cell surface ligand or antigen. The diseases suitable for being treated by the compositions and methods of the disclosure include, but are not limited to, cancers, autoimmune diseases, inflammatory diseases, and infectious diseases. In some embodiments, the disease is a cancer or a chronic infection.

Methods for CAR design, delivery and expression in T cells, and the manufacturing of clinical-grade CAR-T cell populations are known in the art. See, for example, Lee et al., Clin Cancer Res (2012) 18(10):2780-90, hereby incorporated by reference in its entirety. For example, the engineered CARs may be introduced into T cells using retroviruses, which efficiently and stably integrate a nucleic acid sequence encoding the chimeric antigen receptor into the target cell genome. An exemplary method is described in Example 2 below.

Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, Cas12a (Cpf1), Cas13a (C2c2), Cas13b, Cas13d, CasF, CasG, CasH, Csyl, Csy2, Csy3, Cse1 (or CasA), Cse2 (or CasB), Cse3 (or CasE), CasX, CasY, Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.).

In some embodiments, a recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV requires that the following components are present within a single cell (denoted herein as a packaging cell): a rAAV genome, AAV rep and cap genes separate from (e.g., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, international patent application publication number WO 01/83692.

The CAR-T cells, once they have been expanded ex vivo in response to, for example, an autoimmune disease antigen, can be reinfused into the subject in a therapeutically effective amount.

The precise amount of CAR T cells to be administered can be determined by a physician with consideration of individual differences in age, weight, extent of disease and condition of the subject.

Administration of T cell therapies may be defined by number of total cells per infusion or number of cells per kilogram of body weight, especially for pediatric subjects (e.g., patients). As T cells replicate and expand after transfer, the administered cell dose may not resemble the final steady-state number of cells. In some embodiments, a pharmaceutical composition including the CAR T cells of the present disclosure may be administered at a dosage of 104 to 1010 total cells. In another embodiment, a pharmaceutical composition including the CAR T cells of the present disclosure may be administered at a dosage of 103 to 108 cells/kg body weight, including all integer values within those ranges.

Compositions including the CAR T cells of the present disclosure may also be administered multiple times at these dosages. The cells can be administered by using infusion techniques that are known in the art (see, for example, Rosenberg et al., New Engl J Med, (1988) 319:1676). The optimal dosage and treatment regimen for a particular subject can be determined by one skilled in the art by monitoring the subject for signs of disease and adjusting the treatment accordingly.

In some embodiments, administration of any of the compositions embodied herein, for the treatment of, for example, an autoimmune or inflammatory disease, can be combined with other cell-based therapies, for example, stem cells, antigen presenting cells, pancreatic islets etc.

The composition of the present disclosure may be prepared in a manner known in the art and in a manner suitable for parenteral administration to mammals, particularly humans, including a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the present disclosure may also include other supplementary physiologically active agents.

The carrier must be pharmaceutically “acceptable” in the sense of being compatible with the other ingredients of the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the CAR-T cells. In general, the compositions are prepared by uniformly and intimately bringing into association any active ingredients with liquid carriers.

In some embodiments, the composition is suitable for parenteral administration. In another embodiment, the composition is suitable for intravenous administration.

Compositions suitable for parenteral administration include aqueous and nonaqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

### Additional Therapies

As discussed above, any one of the compositions as disclosed herein, e.g., the chimeric receptors, recombinant nucleic acids, recombinant cells, cell cultures, and pharmaceutical compositions described herein can be administered to a subject in need thereof as a single therapy (e.g., monotherapy). In addition or alternatively, in some embodiments of the disclosure, the chimeric receptors, recombinant nucleic acids, recombinant cells, cell cultures, and pharmaceutical compositions described herein can be administered to the subject in combination with one or more additional therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

The present disclosure also contemplates the combination of the composition of the disclosure with other drugs and/or in addition to other treatment regimens or modalities such as surgery. When the composition of the present disclosure is used in combination with known therapeutic agents the combination may be administered either in sequence (either continuously or broken up by periods of no treatment) or concurrently or as an admixture. In the case of, for example, autoimmune diseases, treatment includes administering to the subject the compositions embodied herein, e.g. autologous T cells transduced or contacted with a CAR embodied herein and one or more anti-inflammatory agents and/or therapeutic agents. The anti-inflammatory agents include one or more antibodies which specifically bind to pro-inflammatory cytokines, e.g., pro-inflammatory cytokines such as IL-1, TNF, IL-6, GM-CSF, and IFN-γ. In some embodiments, the antibodies are anti-TNFα, anti-IL-6 or combinations thereof. In some embodiments, one or more agents, other than antibodies can be administered which decrease pro-inflammatory cytokines, e.g. non-steroidal anti-inflammatory drugs (NSAIDs). Any combination of antibodies and one or more agents can be administered which decrease pro-inflammatory cytokines.

Treatment in combination is also contemplated to encompass the treatment with either the composition of the disclosure followed by a known treatment, or treatment with a known agent followed by treatment with the composition of the disclosure, for example, as maintenance therapy. For example, in the treatment of autoimmune diseases, excessive and prolonged activation of immune cells, such as T and B lymphocytes, and overexpression of the master pro-inflammatory cytokine tumor necrosis factor alpha (TNF), together with other mediators such as interleukin-6 (IL-6), interleukin-1 (IL-1), and interferon gamma (IFN-γ), play a central role in the pathogenesis of autoimmune inflammatory responses in rheumatoid arthritis (RA), inflammatory bowel disease (IBD), Crohn's disease (CD), and ankylosing spondylitis (AS).

Non-steroidal anti-inflammatory drugs (NSAIDs), glucocorticoids, disease-modifying anti-rheumatic drugs (DMARDs) are traditionally used in the treatment of autoimmune inflammatory diseases. NSAIDs and glucocorticoids are effective in the alleviation of pain and inhibition of inflammation, while DMARDs have the capacity of reducing tissue and organ damage caused by inflammatory responses. More recently, treatment for RA and other autoimmune diseases has been revolutionized with the discovery that TNF is critically important in the development of the diseases. Anti-TNF biologics (such as infliximab, adalimumab, etanercept, golimumab, and certolizumab pepol) have markedly improved the outcome of the management of autoimmune inflammatory diseases.

Non-steroidal anti-inflammatory drugs have the analgesic, antipyretic, and anti-inflammatory effect, frequently used for the treatment of conditions like arthritis and headaches. NSAIDs relieve pain through blocking cyclooxygenase (COX) enzymes. COX promotes the production of prostaglandins, a mediator which causes inflammation and pain. Although NSAIDs have different chemical structures, all of them have the similar therapeutic effect, e.g., inhibition of autoimmune inflammatory responses. In general, NSAIDs can be divided into two broad categories: traditional non-selective NSAIDs and selective cyclooxygenase-2 (COX-2) inhibitors (For a review, see, P. Li et al., Front Pharmacol (2017) 8:460).

In addition to anti-TNF agents, the biologics targeting other proinflammatory cytokines or immune competent molecules have also been extensively studied and actively developed. For example, abatacept, a fully humanized fusion protein of extracellular domain of CTLA-4 and Fc fraction of IgG1, has been approved for the RA patients with inadequate response to anti-TNF therapy. The major immunological mechanism of abatacept is selective inhibition of co-stimulation pathway (CD80 and CD86) and activation of T cells. Tocilizumab, a humanized anti-IL-6 receptor monoclonal antibody was approved for RA patients intolerant to DMARDs and/or anti-TNF biologics. This therapeutic mAb blocks the transmembrane signaling of IL-6 through binding with soluble and membrane forms of IL-6 receptor. Biological drugs targeting IL-1 (anakinra), Th1 immune responses (IL-12/IL-23, ustekinumab), Th17 immune responses (IL-17, secukinumab) and CD20 (rituximab) have also been approved for the treatment of autoimmune diseases (For a review see, P. Li et al., Front Pharmacol (2017) 8:460).

Accordingly, in some embodiments, the methods of the disclosure include administration of a composition disclosed herein to a subject individually as a single therapy (e.g., monotherapy). In some embodiments, a composition of the disclosure is administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Methods for Modulating an Activity of a Cell

In another aspect, provided herein are various methods for modulating an activity of a cell. The methods include the steps of: (a) providing an effective amount of any of the recombinant cells provided herein, and (b) contacting it with a selected ligand, wherein binding of the selected ligand to the extracellular ligand-binding domain induces cleavage of a ligand-inducible proteolytic cleavage site and releases the intracellular domain comprising the intracellular signaling domain and the transcriptional regulator, wherein the released intracellular signaling domain and the transcriptional regulator modulates an activity of the recombinant cell. One skilled in the art upon reading the present disclosure will appreciate that the disclosed methods can be carried out in vivo, ex vivo, or in vitro.

Non-limiting exemplary cellular activities that can be modulated using the methods provide herein include, but are not limited to, gene expression, proliferation, apoptosis, non-apoptotic death, differentiation, dedifferentiation, migration, secretion of a gene product, cellular adhesion, and cytolytic activity.

In some embodiments, the released transcriptional regulator modulates expression of a gene product of the cell. In some embodiments, the released transcriptional regulator modulates expression of a heterologous gene product in the cell. A heterologous gene product is one that is not normally found in the native cell, e.g., not normally produced by the cell. For example, the cell can be genetically modified with a nucleic acid including a nucleotide sequence encoding the heterologous gene product.

In some embodiments, the heterologous gene product is a secreted gene product. In some embodiments, the heterologous gene product is a cell surface gene product. In some cases, the heterologous gene product is an intracellular gene product. In some embodiments, the released transcriptional regulator simultaneously modulates expression of two or more heterologous gene products in the cell.

In some embodiments, the heterologous gene product in the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen-derived protein, a proliferation inducer, a receptor, an RNA guided nuclease, a site-specific nuclease, a T-cell receptor (TCR), a chimeric antigen receptor (CAR), a toxin, a toxin-derived protein, a transcriptional regulator, a transcriptional activator, a transcriptional repressor, a translation regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

In some embodiments, the released transcriptional regulator modulates differentiation of the cell, and wherein the cell is an immune cell, a stem cell, a progenitor cell, or a precursor cell.

The chimeric receptors of the disclosure provide a higher degree of expression than a standard SynNotch receptor, when using identical binding domains and ICDs. Depending on the ligand/binding domain pair and their affinity, the chimeric receptor s or Hinge-Notch receptors of the disclosure can provide expression enhancement of about 10%, about 15%, about 20%, about 25%, about 30%, about 35%, about 40%, about 45%, or about 50% higher than a corresponding SynNotch receptor.

Additionally, the chimeric receptors of the disclosure can provide transcriptional regulation that responds to the degree of T cell activation, independent of ligand binding. For example, when expressed in a T cell, some receptors of the disclosure provide a stronger ligand-induced signal when the T-cell is activated as compared to the ligand-induced signal when the T-cell is not activated. This permits additional flexibility in use, for example in cases where it is desired to enhance or suppress a T cell response when activated despite the absence of the chimeric receptor ligand.

### Systems and Kits

Also provided herein are systems and kits including the chimeric receptor s, Hinge-Notch receptors, recombinant nucleic acids, recombinant cells, or pharmaceutical compositions provided and described herein as well as written instructions for making and using the same. For example, provided herein, in some embodiments, are systems and/or kits that include one or more of: an chimeric receptor as described herein, a Hinge-Notch receptor as described herein, a recombinant nucleic acids as described herein, a recombinant cell as described herein, or a pharmaceutical composition as described herein. In some embodiments, the systems and/or kits of the disclosure further include one or more syringes (including pre-filled syringes) and/or catheters (including pre-filled syringes) used to administer one any of the provided chimeric receptor s, Hinge-Notch receptors, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a health condition (e.g., disease) in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### Example 1: Design of Hybrid SynNotch Car Circuits

This Example shows the design and construction of the exemplary hybrid SynNotch CARs provided herein.

Intracellular domains containing the appropriate costimulatory domain, CD3zeta domain, Gal4-VP64 and GS linkers were synthesized as gene fragments from Twist. Receptors were built by fusing the CD19, BCMA or ALPPL2 targeting scFv to the corresponding receptor scaffold and intracellular tail. All receptors contain an n-terminal CD8a signal peptide (MALPVTALLLPLALLLHAARP, SEQ ID NO: 69) for membrane targeting and a flag-tag (DYKDDDDK, SEQ ID NO: 70) for easy determination of surface expression with α-flag PE (Biolegend 637310). The receptors were cloned into a modified pHR′SIN:CSW vector containing a PGK promoter for all primary T cell experiments.

The pHR′SIN:CSW vector was also modified to make the response element plasmids. Five copies of the Gal4 DNA binding domain target sequence (GGAGCACTGTCCTCCGAACG, SEQ ID NO: 71) were cloned 5′ to a minimal pybTATA promoter. Also included in the response element plasmids was a PGK promoter that constitutively drives mCitrine expression to easily identify transduced T cells. For all inducible BFP vectors, BFP was cloned via a BamHI site in the multiple cloning site 3′ to the Gal4 response elements. All constructs were cloned via In-fusion cloning (Clontech #ST0345).

FIG. 1A shows a diagram of all possible intracellular domain configurations. FIG. 1B shows a detailed diagram of hybrid SynNotch CAR domains. Further, without being bound by theory, FIG. 1C illustrates the principle of short term proximal and long term transcriptional signaling induced by hybrid SynNotch CARs.

The components of the hybrid SynNotch CARs comprising a 4-1BB costimulatory domain are described in Table 1 below. The N-JMDs of the hybrid SynNotch CARs tested here included a truncated form of the CD8a hinge that is composed of an N-terminal fragment of the typical CD8a Hinge domain.

TABLE 1
Receptor components description for 4-1BB hybrid SynNotch CARs.

| Plasmid | | | N- | | | | | |
|---|---|---|---|---|---|---|---|---|
| ID | Receptor description | ECD | JMD | TMD | STS | ICD 1 | ICD 2 | ICD 3 |
| pRL_2073 | 4-1BB hybrid SynNotch | CD8a signal | CD8a | Notch | Notch | 4-1BB | Gal4, | CD3zeta |
| | CAR Configuration 1: | peptide, | Hinge | 1 | 2 | | VP64 | |
| | Hinge Notch Receptor | myc-tag, | | | | | | |
| | Scaffold with addition of 4- | anti-CD19 | | | | | | |
| | 1BB and CD3zeta | scFv | | | | | | |
| | Intracellular Domains, | | | | | | | |
| | configured with 4-1BB first, | | | | | | | |
| | followed by Gal4VP64 then | | | | | | | |
| | CD3zeta. | | | | | | | |
| pRL_2074 | 4-1BB hybrid SynNotch | CD8a signal | CD8a | Notch | Notch | CD3zeta | 4-1BB | Gal4, |
| | CAR Configuration 2: | peptide, | Hinge | 1 | 2 | | | VP64 |
| | Hinge Notch Receptor | myc-tag, | | | | | | |
| | Scaffold with addition of 4- | anti-CD19 | | | | | | |
| | 1BB and CD3zeta | scFv | | | | | | |
| | Intracellular Domains, | | | | | | | |
| | configured with CD3zeta | | | | | | | |
| | first, followed by 4-1BB | | | | | | | |
| | then Gal4VP64 | | | | | | | |
| pRL_2075 | 4-1BB hybrid SynNotch | CD8a signal | CD8a | Notch | Notch | CD3zeta | Gal4, | 4-1BB |
| | CAR Configuration 3: | peptide, | Hinge | 1 | 2 | | VP64 | |
| | Hinge Notch Receptor | myc-tag, | | | | | | |
| | Scaffold with addition of 4- | anti-CD19 | | | | | | |
| | 1BB and CD3zeta | scFv | | | | | | |
| | Intracellular Domains, | | | | | | | |
| | configured with CD3zeta | | | | | | | |
| | first, followed by | | | | | | | |
| | Gal4VP64, then 4-1BB | | | | | | | |
| pRL_2076 | 4-1BB hybrid SynNotch | CD8a signal | CD8a | Notch | Notch | 4-1BB | CD3zeta | Gal4, |
| | CAR Configuration 4: | peptide, | Hinge | 1 | 2 | | | VP64 |
| | Hinge Notch Receptor | myc-tag, | | | | | | |
| | Scaffold with addition of 4- | anti-CD19 | | | | | | |
| | 1BB and CD3zeta | scFv | | | | | | |
| | Intracellular Domains, | | | | | | | |
| | configured with 4-1BB first, | | | | | | | |
| | followed by CD3zeta then | | | | | | | |
| | Gal4VP64 | | | | | | | |
| pRL_2077 | 4-1BB hybrid SynNotch | CD8a signal | CD8a | Notch | Notch | Gal4, | 4-1BB | CD3zeta |
| | CAR Configuration 5: | peptide, | Hinge | 1 | 2 | VP64 | | |
| | Hinge Notch Receptor | myc-tag, | | | | | | |
| | Scaffold with addition of 4- | anti-CD19 | | | | | | |
| | 1BB and CD3zeta | scFv | | | | | | |
| | Intracellular Domains, | | | | | | | |
| | configured with Gal4VP64 | | | | | | | |
| | first, followed by 4-1BB, | | | | | | | |
| | then CD3zeta | | | | | | | |
| pRL_2078 | 4-1BB hybrid SynNotch | CD8a signal | CD8a | Notch | Notch | Gal4, | CD3zeta | 4-1BB |
| | CAR Configuration 6: | peptide, | Hinge | 1 | 2 | VP64 | | |
| | Hinge Notch Receptor | myc-tag, | | | | | | |
| | Scaffold with addition of 4- | anti-CD19 | | | | | | |
| | 1BB and CD3zeta | scFv | | | | | | |
| | Intracellular Domains, | | | | | | | |
| | configured with Gal4VP64 | | | | | | | |
| | first, followed by CD3zeta, | | | | | | | |
| | then 4-1BB | | | | | | | |
| pRL_2097 | 4-1BB hybrid SynNotch | CD8a signal | CD8a | Notch | Notch | 4-1BB | Gal4, | CD3zeta |
| | CAR Configuration 1 using | peptide, | Hinge | 1 | 2 | | VP64 | |
| | Flag tag instead of Myc Tag | flag-tag, | | | | | | |
| | | anti-CD19 | | | | | | |
| | | scFv | | | | | | |
| pRL_2098 | 4-1BB hybrid SynNotch | CD8a signal | CD8a | Notch | Notch | CD3zeta | 4-1BB | Gal4, |
| | CAR Configuration 2 using | peptide, | Hinge | 1 | 2 | | | VP64 |
| | Flag tag instead of Myc Tag | flag-tag, | | | | | | |
| | | anti-CD19 | | | | | | |
| | | scFv | | | | | | |
| pRL_2099 | 4-1BB hybrid SynNotch | CD8a signal | CD8a | Notch | Notch | CD3zeta | Gal4, | 4-1BB |
| | CAR Configuration 3 using | peptide, | Hinge | 1 | 2 | | VP64 | |
| | Flag tag instead of Myc Tag | flag-tag, | | | | | | |
| | | anti-CD19 | | | | | | |
| | | scFv | | | | | | |
| pRL_2100 | 4-1BB hybrid SynNotch | CD8a signal | CD8a | Notch | Notch | 4-1BB | CD3zeta | Gal4, |
| | CAR Configuration 4 using | peptide, | Hinge | 1 | 2 | | | VP64 |
| | Flag tag instead of Myc Tag | flag-tag, | | | | | | |
| | | anti-CD19 | | | | | | |
| | | scFv | | | | | | |
| pRL_2101 | 4-1BB hybrid SynNotch | CD8a signal | CD8a | Notch | Notch | Gal4, | 4-1BB | CD3zeta |
| | CAR Configuration 5 using | peptide, | Hinge | 1 | 2 | VP64 | | |
| | Flag tag instead of Myc Tag | flag-tag, | | | | | | |
| | | anti-CD19 | | | | | | |
| | | scFv | | | | | | |
| pRL_2102 | 4-1BB hybrid SynNotch | CD8a signal | CD8a | Notch | Notch | Gal4, | CD3zeta | 4-1BB |
| | CAR Configuration 6 using | peptide, | Hinge | 1 | 2 | VP64 | | |
| | Flag tag instead of Myc Tag | flag-tag, | | | | | | |
| | | anti-CD19 | | | | | | |
| | | scFv | | | | | | |
| pRL_2140 | 4-1BB hybrid SynNotch | CD8a signal | CD8a | Notch | Notch | noSTS | CD3zeta | Gal4, |
| | CAR Configuration 4 using | peptide, | Hinge | 1 | 2 | 4-1BB | | VP64 |
| | 4-1BB that has STS | flag-tag, | | | | | | |
| | removed (removed Uniprot | anti-CD19 | | | | | | |
| | Q07011: AA214-219 from | scFv | | | | | | |
| | pRL_2100) | | | | | | | |
| pRL_2142 | 4-1BB hybrid SynNotch | CD8a signal | CD8a | Notch | Notch | truncated | CD3zeta | Gal4, |
| | CAR Configuration 4 with | peptide, | Hinge | 1 | 2 | 4- | | VP64 |
| | truncated 4-1BB (removed | flag-tag, | | | | 1BB | | |
| | Uniprot Q07011: AA214- | anti-CD19 | | | | | | |
| | 230 from pRL_2100) | scFv | | | | | | |
| pRL_2184 | 4-1BB hybrid SynNotch | CD8a signal | CD8a | Notch | Notch | Truncated | CD3zeta | Gal4V |
| | CAR Configuration 4 with | peptide, | Hinge | 1 | 2 | 4- | | P64 |
| | two truncated 4-1BB | flag-tag, | | | | 1BB, | | |
| | fragments | anti-CD19 | | | | truncated | | |
| | | scFv | | | | 4- | | |
| | | | | | | 1BB | | |
| pRL_2185 | 4-1BB hybrid SynNotch | CD8a signal | CD8a | Notch | Notch | Truncated | CD3zeta | Gal4V |
| | CAR Configuration 4 with | peptide, | Hinge | 1 | 2 | 4- | | P64 |
| | three truncated 4-1BB | flag-tag, | | | | 1BB, | | |
| | fragments | anti-CD19 | | | | truncated | | |
| | | scFv | | | | 4- | | |
| | | | | | | 1BB, | | |
| | | | | | | Truncated | | |
| | | | | | | 4- | | |
| | | | | | | 1BB | | |
| pRL_2186 | 4-1BB hybrid SynNotch | CD8a signal | CD8a | Notch | Notch | Minimal | CD3zeta | Gal4V |
| | CAR Configuration 4 with | peptide, | Hinge | 1 | 2 | 4-1BB | | P64 |
| | minimal 4-1BB (removed | flag-tag, | | | | | | |
| | Uniprot Q07011: AA214- | anti-CD19 | | | | | | |
| | 230 and Uniprot Q07011: | scFv | | | | | | |
| | AA243-255 from | | | | | | | |
| | pRL_2100) | | | | | | | |
| pRL_2232 | 41BB Hybrid SynNotch | CD8a signal | CD8a | Notch | Notch | truncated | CD3zeta | Gal4, |
| | CAR Configuration 4 with | peptide, | Hinge | 1 | 2 | 41BB | | VP64 |
| | truncated 41BB (removed | flag-tag, | | | | | | |
| | Uniprot Q07011: AA214- | anti-BCMA | | | | | | |
| | 230 from pRL_2100) | scFv | | | | | | |
| pRL_2234 | 41BB Hybrid SynNotch | CD8a signal | CD8a | Notch | Notch | truncated | CD3zeta | Gal4, |
| | CAR Configuration 4 with | peptide, | Hinge | 1 | 2 | 41BB | | VP64 |
| | truncated 41BB (removed | flag-tag, | | | | | | |
| | Uniprot Q07011: AA214- | anti- | | | | | | |
| | 230 from pRL_2100) | ALPPL2 | | | | | | |
| | | scFv | | | | | | |

In addition, the reference sequences from which each of the components listed in Table 1 is derived from are listed in Table 2 below.

TABLE 2
sequence references for 4-1BB hybrid SynNotch CARs.

| | | | N- | | ICD | | | |
|---|---|---|---|---|---|---|---|---|
| PlasmidID | Receptor | ECD | JMD | TMD | (STS) | ICD 1 | ICD 2 | ICD 3 |
| pRL_2073 | antiCD19scFv- | Uniprot P01732: | Uniprot | Uniprot | Uniprot | Uniprot | Uniprot | Uniprot |
| | CD8Hinge2- | AA1-21, Uniprot | P01732: | P46531: | Q04721: | Q07011: | P04386: | P20963-3: |
| | Notch1TMD- | P01106: AA410- | AA138- | AA1736- | AA1701- | AA214-255 | AA1-147, | AA52-164 |
| | Notch2STS-4- | 419, FMC63 | 164 | 1757 | 1706 | | Uniprot | |
| | 1BB- | scFv sequence | | | | | P06492: | |
| | Gal4VP64- | (ncbi.nlm.nih.gov/ | | | | | AA437-447 | |
| | CD3zeta | pubmed/9566763/) | | | | | plus GS linker | |
| | | | | | | | altogether | |
| | | | | | | | repeated 4 | |
| | | | | | | | times | |
| pRL_2074 | antiCD19scFv- | Uniprot P01732: | Uniprot | Uniprot | Uniprot | Uniprot | Uniprot | Uniprot |
| | CD8Hinge2- | AA1-21, Uniprot | P01732: | P46531: | Q04721: | P20963-3: | Q07011: | P04386: |
| | Notch1TMD- | P01106: AA410- | AA138- | AA1736- | AA1701- | AA52-164 | AA214-255 | AA1-147, |
| | Notch2STS- | 419, FMC63 | 164 | 1757 | 1706 | | | Uniprot |
| | CD3zeta-4- | scFv sequence | | | | | | P06492: |
| | 1BB-Gal4VP64 | (ncbi.nlm.nih.gov/ | | | | | | AA437-447 |
| | | pubmed/9566763/) | | | | | | plus GS linker |
| | | | | | | | | altogether |
| | | | | | | | | repeated 4 |
| | | | | | | | | times |
| pRL_2075 | antiCD19scFv- | Uniprot P01732: | Uniprot | Uniprot | Uniprot | Uniprot | Uniprot | Uniprot |
| | CD8Hinge2- | AA1-21, Uniprot | P01732: | P46531: | Q04721: | P20963-3: | P04386: | Q07011: |
| | Notch1TMD- | P01106: AA410- | AA138- | AA1736- | AA1701- | AA52-164 | AA1-147, | AA214-255 |
| | Notch2STS- | 419, FMC63 | 164 | 1757 | 1706 | | Uniprot | |
| | CD3zeta- | scFv sequence | | | | | P06492: | |
| | Gal4VP64-4- | (ncbi.nlm.nih.gov/ | | | | | AA437-447 | |
| | 1BB | pubmed/9566763/) | | | | | plus GS linker | |
| | | | | | | | altogether | |
| | | | | | | | repeated 4 | |
| | | | | | | | times | |
| pRL_2076 | antiCD19scFv- | Uniprot P01732: | Uniprot | Uniprot | Uniprot | Uniprot | Uniprot | Uniprot |
| | CD8Hinge2- | AA1-21, Uniprot | P01732: | P46531: | Q04721: | Q07011: | P20963-3: | P04386: |
| | Notch1TMD- | P01106: AA410- | AA138- | AA1736- | AA1701- | AA214-255 | AA52-164 | AA1-147, |
| | Notch2STS-4- | 419, FMC63 | 164 | 1757 | 1706 | | | Uniprot |
| | 1BB-CD3zeta- | scFv sequence | | | | | | P06492: |
| | Gal4VP64 | (ncbi.nlm.nih.gov/ | | | | | | AA437-447 |
| | | pubmed/9566763/) | | | | | | plus GS linker |
| | | | | | | | | altogether |
| | | | | | | | | repeated 4 |
| | | | | | | | | times |
| pRL_2077 | antiCD19scFv- | Uniprot P01732: | Uniprot | Uniprot | Uniprot | Uniprot | Uniprot | Uniprot |
| | CD8Hinge2- | AA1-21, Uniprot | P01732: | P46531: | Q04721: | P04386: | Q07011: | P20963-3: |
| | Notch1TMD- | P01106: AA410- | AA138- | AA1736- | AA1701- | AA1-147, | AA214-255 | AA52-164 |
| | Notch2STS- | 419, FMC63 | 164 | 1757 | 1706 | Uniprot | | |
| | Gal4VP64-4- | scFv sequence | | | | P06492: | | |
| | 1BB-CD3zeta | (ncbi.nlm.nih.gov/ | | | | AA437-447 | | |
| | | pubmed/9566763/) | | | | plus GS linker | | |
| | | | | | | altogether | | |
| | | | | | | repeated 4 | | |
| | | | | | | times | | |
| pRL_2078 | antiCD19scFv- | Uniprot P01732: | Uniprot | Uniprot | Uniprot | Uniprot | Uniprot | Uniprot |
| | CD8Hinge2- | AA1-21, Uniprot | P01732: | P46531: | Q04721: | P04386: | P20963-3: | Q07011: |
| | Notch1TMD- | P01106: AA410- | AA138- | AA1736- | AA1701- | AA1-147, | AA52-164 | AA214-255 |
| | Notch2STS- | 419, FMC63 | 164 | 1757 | 1706 | Uniprot | | |
| | Gal4VP64- | scFv sequence | | | | P06492: | | |
| | CD3zeta-4- | (ncbi.nlm.nih.gov/ | | | | AA437-447 | | |
| | 1BB | pubmed/9566763/) | | | | plus GS linker | | |
| | | | | | | altogether | | |
| | | | | | | repeated 4 | | |
| | | | | | | times | | |
| pRL_2097 | antiCD19scFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | Q07011: | P04386: | P20963-3: |
| | Notch1TMD- | TAG?, FMC63 | AA138- | AA1736- | AA1701- | AA214-255 | AA1-147, | AA52-164 |
| | Notch2STS-4- | scFv sequence | 164 | 1757 | 1706 | | Uniprot | |
| | 1BB- | (ncbi.nlm.nih.gov/ | | | | | P06492: | |
| | Gal4VP64- | pubmed/9566763/) | | | | | AA437-447 | |
| | CD3zeta | | | | | | plus GS linker | |
| | | | | | | | altogether | |
| | | | | | | | repeated 4 | |
| | | | | | | | times | |
| pRL_2098 | antiCD19scFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | P20963-3: | Q07011: | P04386: |
| | Notch1TMD- | TAG?, FMC63 | AA138- | AA1736- | AA1701- | AA52-164 | AA214-255 | AA1-147, |
| | Notch2STS- | scFv sequence | 164 | 1757 | 1706 | | | Uniprot |
| | CD3zeta-4- | (ncbi.nlm.nih.gov/ | | | | | | P06492: |
| | 1BB-Gal4VP64 | pubmed/9566763/) | | | | | | AA437-447 |
| | | | | | | | | plus GS linker |
| | | | | | | | | altogether |
| | | | | | | | | repeated 4 |
| | | | | | | | | times |
| pRL_2099 | antiCD19scFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | P20963-3: | P04386: | Q07011: |
| | Notch1TMD- | TAG?, FMC63 | AA138- | AA1736- | AA1701- | AA52-164 | AA1-147, | AA214-255 |
| | Notch2STS- | scFv sequence | 164 | 1757 | 1706 | | Uniprot | |
| | CD3zeta- | (ncbi.nlm.nih.gov/ | | | | | P06492: | |
| | Gal4VP64-4- | pubmed/9566763/) | | | | | AA437-447 | |
| | 1BB | | | | | | plus GS linker | |
| | | | | | | | altogether | |
| | | | | | | | repeated 4 | |
| | | | | | | | times | |
| pRL_2100 | antiCD19scFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | Q07011: | P20963-3: | P04386: |
| | Notch1TMD- | TAG?, FMC63 | AA138- | AA1736- | AA1701- | AA214-255 | AA52-164 | AA1-147, |
| | Notch2STS-4- | scFv sequence | 164 | 1757 | 1706 | | | Uniprot |
| | 1BB-CD3zeta- | (ncbi.nlm.nih.gov/ | | | | | | P06492: |
| | Gal4VP64 | pubmed/9566763/) | | | | | | AA437-447 |
| | | | | | | | | plus GS linker |
| | | | | | | | | altogether |
| | | | | | | | | repeated 4 |
| | | | | | | | | times |
| pRL_2101 | antiCD19scFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | P04386: | Q07011: | P20963-3: |
| | Notch1TMD- | TAG?, FMC63 | AA138- | AA1736- | AA1701- | AA1-147, | AA214-255 | AA52-164 |
| | Notch2STS- | scFv sequence | 164 | 1757 | 1706 | Uniprot | | |
| | Gal4VP64-4- | (ncbi.nlm.nih.gov/ | | | | P06492: | | |
| | 1BB-CD3zeta | pubmed/9566763/) | | | | AA437-447 | | |
| | | | | | | plus GS linker | | |
| | | | | | | altogether | | |
| | | | | | | repeated 4 | | |
| | | | | | | times | | |
| pRL_2102 | antiCD19scFv- | Uniprot P01732: | Uniprot | Uniprot | Uniprot | Uniprot | Uniprot | Uniprot |
| | CD8Hinge2- | AA1-21, FLAG | P01732 | P46531: | Q04721: | P04386: | P20963-3: | Q07011: |
| | Notch1TMD- | TAG?, FMC63 | AA138- | AA1736- | AA1701- | AA1-147, | AA52-164 | AA214-255 |
| | Notch2STS- | scFv sequence | 164 | 1757 | 1706 | Uniprot | | |
| | Gal4VP64- | (ncbi.nlm.nih.gov/ | | | | P06492: | | |
| | CD3zeta-4- | pubmed/9566763/) | | | | AA437-447 | | |
| | 1BB | | | | | plus GS linker | | |
| | | | | | | altogether | | |
| | | | | | | repeated 4 | | |
| | | | | | | times | | |
| pRL_2140 | antiCD19scFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | Q07011: | P20963-3: | P04386: |
| | Notch1TMD- | TAG?, FMC63 | AA138- | AA1736- | AA1701- | AA220-255 | AA52-164 | AA1-147, |
| | Notch2STS- | scFv sequence | 164 | 1757 | 1706 | | | Uniprot |
| | noSTS4-1BB- | (ncbi.nlm.nih.gov/ | | | | | | P06492: |
| | CD3zeta- | pubmed/9566763/) | | | | | | AA437-447 |
| | Gal4VP64 | | | | | | | plus GS linker |
| | | | | | | | | altogether |
| | | | | | | | | repeated 4 |
| | | | | | | | | times |
| pRL_2142 | antiCD19scFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | Q07011: | P20963-3: | P04386: |
| | Notch1TMD- | TAG?, FMC63 | AA138- | AA1736- | AA1701- | AA231-255 | AA52-164 | AA1-147, |
| | Notch2STS- | scFv sequence | 164 | 1757 | 1706 | | | Uniprot |
| | trunc4-1BB- | (ncbi.nlm.nih.gov/ | | | | | | P06492: |
| | CD3zeta- | pubmed/9566763/) | | | | | | AA437-447 |
| | Gal4VP64 | | | | | | | plus GS linker |
| | | | | | | | | altogether |
| | | | | | | | | repeated 4 |
| | | | | | | | | times |
| pRL_2184 | antiCD19scFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | Q07011 | P20963-3: | P04386: |
| | Notch1TMD- | TAG?, FMC63 | AA138- | AA1736- | AA1701- | AA231-255, | AA52-164 | AA1-147, |
| | Notch2STS- | scFv sequence | 164 | 1757 | 1706 | Uniprot | | Uniprot |
| | trunc4- | (ncbi.nlm.nih.gov/ | | | | Q07011: | | P06492: |
| | 1BBtrunc4- | pubmed/9566763/) | | | | AA231-255 | | AA437-447 |
| | 1BB-CD3zeta- | | | | | | | plus GS linker |
| | Gal4VP64 | | | | | | | altogether |
| | | | | | | | | repeated 4 |
| | | | | | | | | times |
| pRL_2185 | antiCD19scFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | Q07011 | P20963-3: | P04386: |
| | Notch1TMD- | TAG?, FMC63 | AA138- | AA1736- | AA1701- | AA231-255, | AA52-164 | AA1-147, |
| | Notch2STS- | scFv sequence | 164 | 1757 | 1706 | Uniprot | | Uniprot |
| | trunc4- | (ncbi.nlm.nih.gov/ | | | | Q07011: | | P06492: |
| | 1BBtrunc4- | pubmed/9566763/) | | | | AA231-255, | | AA437-447 |
| | 1BBtrunc4- | | | | | Uniprot | | plus GS linker |
| | 1BB-CD3zeta- | | | | | Q07011: | | altogether |
| | Gal4VP64 | | | | | AA231-255 | | repeated 4 |
| | | | | | | | | times |
| pRL_2186 | antiCD19scFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | Q07011: | P20963-3: | P04386: |
| | Notch1TMD- | TAG?, FMC63 | AA138- | AA1736- | AA1701- | AA231-242 | AA52-164 | AA1-147, |
| | Notch2STS- | scFv sequence | 164 | 1757 | 1706 | | | Uniprot |
| | min4-1BB- | (ncbi.nlm.nih.gov/ | | | | | | P06492: |
| | CD3zeta- | pubmed/9566763/) | | | | | | AA437-447 |
| | Gal4VP64 | | | | | | | plus GS linker |
| | | | | | | | | altogether |
| | | | | | | | | repeated 4 |
| | | | | | | | | times |
| pRL_2232 | antiBCMAscFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | Q07011: | P20963-3: | P04386: |
| | Notch1TMD- | TAG?, BCMA- | AA138- | AA1736- | AA1701- | AA231-255 | AA52-164 | AA1-147, |
| | Notch2STS- | 50 scFv | 164 | 1757 | 1706 | | | Uniprot |
| | trunc41BB- | sequence (U.S. | | | | | | P06492: |
| | CD3z- | Pat. No. | | | | | | AA437-447 |
| | Gal4VP64 | 2017/01834.18 | | | | | | plus GS linker |
| | | A1) | | | | | | altogether |
| | | | | | | | | repeated 4 |
| | | | | | | | | times |
| pRL_2234 | antiALPPL2scFv- | Uniprot P01732: | Uniprot | Uniprot | Uniprot | Uniprot | Uniprot | Uniprot |
| | CD8Hinge2- | AA1-21, FLAG | P01732: | P46531: | Q04721: | Q07011: | P20963-3: | P04386: |
| | Notch1TMD- | TAG?, M25 | AA138- | AA1736- | AA1701- | AA231-255 | AA52-164 | AA1-147, |
| | Notch2STS- | FYIA scFv | 164 | 1757 | 1706 | | | Uniprot |
| | trunc41BB- | sequence | | | | | | P06492: |
| | CD3z- | (https:// | | | | | | AA437-447 |
| | Gal4VP64 | pubmed.ncbi.nlm.nih.gov/ | | | | | | plus GS linker |
| | | 32868383/) | | | | | | altogether |
| | | | | | | | | repeated 4 |
| | | | | | | | | times |

In addition, the exemplary hybrid SynNotch CARs comprising a CD28 costimulatory domain are described in Table 3 below.

TABLE 3
Receptor components description for CD28 hybrid SynNotch CARs

| Plasmid | | | | | ICD |
|---|---|---|---|---|---|
| ID | Receptor description | ECD | N-JMD | TMD | (STS) |
| pRL_2080 | CD28 hybrid SynNotch CAR | CD8a | CD8a | Notch | Notch |
| | Configuration 1: Hinge Notch | signal | Hinge | 1 | 2 |
| | Receptor Scaffold with addition of | peptide, | | | |
| | CD28 and CD3zeta Intracellular | myc-tag, | | | |
| | Domains, configured with CD28 | anti-CD19 | | | |
| | first, followed by Gal4VP64 then | scFv | | | |
| | CD3zeta. | | | | |
| pRL_2081 | CD28 hybrid SynNotch CAR | CD8a | CD8a | Notch | Notch |
| | Configuration 2: Hinge Notch | signal | Hinge | 1 | 2 |
| | Receptor Scaffold with addition of | peptide, | | | |
| | CD28 and CD3zeta Intracellular | myc-tag, | | | |
| | Domains, configured with CD3zeta | anti-CD19 | | | |
| | first, followed by CD28 then | scFv | | | |
| | Gal4VP64 | | | | |
| pRL_2082 | CD28 hybrid SynNotch CAR | CD8a | CD8a | Notch | Notch |
| | Configuration 3: Hinge Notch | signal | Hinge | 1 | 2 |
| | Receptor Scaffold with addition of | peptide, | | | |
| | CD28 and CD3zeta Intracellular | myc-tag, | | | |
| | Domains, configured with CD3zeta | anti-CD19 | | | |
| | first, followed by Gal4VP64, then | scFv | | | |
| | CD28 | | | | |
| pRL_2083 | CD28 hybrid SynNotch CAR | CD8a | CD8a | Notch | Notch |
| | Configuration 4: Hinge Notch | signal | Hinge | 1 | 2 |
| | Receptor Scaffold with addition of | peptide, | | | |
| | CD28 and CD3zeta Intracellular | myc-tag, | | | |
| | Domains, configured with CD28 | anti-CD19 | | | |
| | first, followed by CD3zeta then | scFv | | | |
| | Gal4VP64 | | | | |
| pRL_2084 | CD28 hybrid SynNotch CAR | CD8a | CD8a | Notch | Notch |
| | Configuration 5: Hinge Notch | signal | Hinge | 1 | 2 |
| | Receptor Scaffold with addition of | peptide, | | | |
| | CD28 and CD3zeta Intracellular | myc-tag, | | | |
| | Domains, configured with Gal4VP64 | anti-CD19 | | | |
| | first, followed by CD28, then | scFv | | | |
| | CD3zeta | | | | |
| pRL_2085 | CD28 hybrid SynNotch CAR | CD8a | CD8a | Notch | Notch |
| | Configuration 6: Hinge Notch | signal | Hinge | 1 | 2 |
| | Receptor Scaffold with addition of | peptide, | | | |
| | CD28 and CD3zeta Intracellular | myc-tag, | | | |
| | Domains, configured with Gal4VP64 | anti-CD19 | | | |
| | first, followed by CD3zeta, then | scFv | | | |
| | CD28 | | | | |
| pRL_2103 | CD28 hybrid SynNotch CAR | CD8a | CD8a | Notch | Notch |
| | Configuration 1 using Flag tag | signal | Hinge | 1 | 2 |
| | instead of Myc Tag | peptide, | | | |
| | | flag-tag, | | | |
| | | anti-CD19 | | | |
| | | scFv | | | |
| pRL_2104 | CD28 hybrid SynNotch CAR | CD8a | CD8a | Notch | Notch |
| | Configuration 2 using Flag tag | signal | Hinge | 1 | 2 |
| | instead of Myc Tag | peptide, | | | |
| | | flag-tag, | | | |
| | | anti-CD19 | | | |
| | | scFv | | | |
| pRL_2105 | CD28 hybrid SynNotch CAR | CD8a | CD8a | Notch | Notch |
| | Configuration 3 using Flag tag | signal | Hinge | 1 | 2 |
| | instead of Myc Tag | peptide, | | | |
| | | flag-tag, | | | |
| | | anti-CD19 | | | |
| | | scFv | | | |
| pRL_2106 | CD28 hybrid SynNotch CAR | CD8a | CD8a | Notch | Notch |
| | Configuration 4 using Flag tag | signal | Hinge | 1 | 2 |
| | instead of Myc Tag | peptide, | | | |
| | | flag-tag, | | | |
| | | anti-CD19 | | | |
| | | scFv | | | |
| pRL_2107 | CD28 hybrid SynNotch CAR | CD8a | CD8a | Notch | Notch |
| | Configuration 5 using Flag tag | signal | Hinge | 1 | 2 |
| | instead of Myc Tag | peptide, | | | |
| | | flag-tag, | | | |
| | | anti-CD19 | | | |
| | | scFv | | | |
| pRL_2108 | CD28 hybrid SynNotch CAR | CD8a | CD8a | Notch | Notch |
| | Configuration 6 using Flag tag | signal | Hinge | 1 | 2 |
| | instead of Myc Tag | peptide, | | | |
| | | flag-tag, | | | |
| | | anti-CD19 | | | |
| | | scFv | | | |
| pRL_2144 | CD28 Hybrid SynNotch CAR | CD8a | CD8a | Notch | Notch |
| | Configuration 4 using CD28 that has | signal | Hinge | 1 | 2 |
| | STS removed (removed Uniprot | peptide, | | | |
| | P10747: AA180-185 from | flag-tag, | | | |
| | pRL_2106) | anti-CD19 | | | |
| | | scFv | | | |
| pRL_2146 | CD28 Hybrid SynNotch CAR | CD8a | CD8a | Notch | Notch |
| | Configuration 4 using CD28 that is | signal | Hinge | 1 | 2 |
| | truncated (removed Uniprot P10747: | peptide, | | | |
| | AA180-188 from pRL_2106) | flag-tag, | | | |
| | | anti-CD19 | | | |
| | | scFv | | | |
| pRL_2205 | CD28 Hybrid SynNotch CAR | CD8a | CD8a | Notch | Notch |
| | Configuration 4 using CD28 that | signal | Hinge | 1 | 2 |
| | deletes AA sequence TPRRP | peptide, | | | |
| | (removed Uniprot P10747: AA195- | flag-tag, | | | |
| | 199 from pRL_2106) | anti-CD19 | | | |
| | | scFv | | | |
| pRL_2206 | CD28 Hybrid SynNotch CAR | CD8a | CD8a | Notch | Notch |
| | Configuration 4 using CD28 that is | signal | Hinge | 1 | 2 |
| | truncated and deletes AA sequence | peptide, | | | |
| | TPRRP (removed Uniprot P10747: | flag-tag, | | | |
| | AA180-188 and AA195-199 from | anti-CD19 | | | |
| | pRL_2106) | scFv | | | |
| pRL_2207 | CD28 Hybrid SynNotch CAR | CD8a | CD8a | Notch | Notch |
| | Configuration 4 using CD28 that is | signal | Hinge | 1 | 2 |
| | further truncated (removed Uniprot | peptide, | | | |
| | P10747: AA180-199 from | flag-tag, | | | |
| | pRL_2106) | anti-CD19 | | | |
| | | scFv | | | |

| Plasmid | Intracellular | Intracellular | Intracellular |
|---|---|---|---|
| ID | Domain 1 | Domain 2 | Domain 3 |
| pRL_2080 | CD28 | Gal4, VP64 | CD3zeta |
| pRL_2081 | CD3zeta | CD28 | Gal4, VP64 |
| pRL_2082 | CD3zeta | Gal4, VP64 | CD28 |
| pRL_2083 | CD28 | CD3zeta | Gal4, VP64 |
| pRL_2084 | Gal4, VP64 | CD28 | CD3zeta |
| pRL_2085 | Gal4, VP64 | CD3zeta | CD28 |
| pRL_2103 | CD28 | Gal4, VP64 | CD3zeta |
| pRL_2104 | CD3zeta | CD28 | Gal4, VP64 |
| pRL_2105 | CD3zeta | Gal4, VP64 | CD28 |
| pRL_2106 | CD28 | CD3zeta | Gal4, VP64 |
| pRL_2107 | Gal4, VP64 | CD28 | CD3zeta |
| pRL_2108 | Gal4, VP64 | CD3zeta | CD28 |
| pRL_2144 | noSTS CD28 | CD3zeta | Gal4, VP64 |
| pRL_2146 | truncated CD28 | CD3zeta | Gal4, VP64 |
| pRL_2205 | CD28 with | CD3zeta | Gal4, VP64 |
| | TPRRP deletion | | |
| pRL_2206 | truncated CD28 | CD3zeta | Gal4, VP64 |
| | with TPRRP | | |
| | deletion | | |
| pRL_2207 | fully truncated | CD3zeta | Gal4, VP64 |
| | CD28 | | |

Additionally, Table 4 below provides sequence references for receptor components for CD28 hybrid SynNotch CARs.

TABLE 4
Sequence references for CD28 hybrid SynNotch CARs.

| Plasmid | | | N- | | ICD |
|---|---|---|---|---|---|
| ID | Receptor | ECD | JMD | TMD | (STS) |
| pRL_2080 | antiCD19scFv- | Uniprot P01732: AA1-21, Uniprot | Uniprot | Uniprot | Uniprot |
| | CD8Hinge2- | PO1106: AA410-419, FMC63 scFv | P01732: | P46531: | Q04721: |
| | Notch1TMD- | sequence | AA138- | AA1736- | AA1701- |
| | Notch2STS- | (ncbi.nlm.nih.gov/pubmed/9566763/) | 164 | 1757 | 1706 |
| | CD28- | | | | |
| | Gal4VP64- | | | | |
| | CD3zeta | | | | |
| pRL_2081 | antiCD19scFv- | Uniprot P01732: AA1-21, Uniprot | Uniprot | Uniprot | Uniprot |
| | CD8Hinge2- | PO1106: AA410-419, FMC63 scFv | P01732: | P46531: | Q04721: |
| | Notch1TMD- | sequence | AA138- | AA1736- | AA1701- |
| | Notch2STS- | (ncbi.nlm.nih.gov/pubmed/9566763/) | 164 | 1757 | 1706 |
| | CD3zeta-CD28- | | | | |
| | Gal4VP64 | | | | |
| pRL_2082 | antiCD19scFv- | Uniprot P01732: AA1-21, Uniprot | Uniprot | Uniprot | Uniprot |
| | CD8Hinge2- | PO1106: AA410-419, FMC63 scFv | P01732: | P46531: | Q04721: |
| | Notch1TMD- | sequence | AA138- | AA1736- | AA1701- |
| | Notch2STS- | (ncbi.nlm.nih.gov/pubmed/9566763/) | 164 | 1757 | 1706 |
| | CD3zeta- | | | | |
| | Gal4VP64- | | | | |
| | CD28 | | | | |
| pRL_2083 | antiCD19scFv- | Uniprot P01732: AA1-21, Uniprot | Uniprot | Uniprot | Uniprot |
| | CD8Hinge2- | P01106: AA410-419, FMC63 scFv | P01732: | P46531: | Q04721: |
| | Notch1TMD- | sequence | AA138- | AA1736- | AA1701- |
| | Notch2STS- | (ncbi.nlm.nih.gov/pubmed/9566763/) | 164 | 1757 | 1706 |
| | CD28-CD3zeta- | | | | |
| | Gal4VP64 | | | | |
| pRL_2084 | antiCD19scFv- | Uniprot P01732: AA1-21, Uniprot | Uniprot | Uniprot | Uniprot |
| | CD8Hinge2- | PO1106: AA410-419, FMC63 scFv | P01732: | P46531: | Q04721: |
| | Notch1TMD- | sequence | AA138- | AA1736- | AA1701- |
| | Notch2STS- | (ncbi.nlm.nih.gov/pubmed/9566763/) | 164 | 1757 | 1706 |
| | Gal4VP64- | | | | |
| | CD28-CD3zeta | | | | |
| pRL_2085 | antiCD19scFv- | Uniprot P01732: AA1-21, Uniprot | Uniprot | Uniprot | Uniprot |
| | CD8Hinge2- | PO1106: AA410-419, FMC63 scFv | P01732: | P46531: | Q04721: |
| | Notch1TMD- | sequence | AA138- | AA1736- | AA1701- |
| | Notch2STS- | (ncbi.nlm.nih.gov/pubmed/9566763/) | 164 | 1757 | 1706 |
| | Gal4VP64- | | | | |
| | CD3zeta-CD28 | | | | |
| pRL_2103 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (ncbi.nlm.nih.gov/pubmed/9566763/) | AA138- | AA1736- | AA1701- |
| | Notch2STS- | | 164 | 1757 | 1706 |
| | CD28- | | | | |
| | Gal4VP64- | | | | |
| | CD3zeta | | | | |
| pRL_2104 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (ncbi.nlm.nih.gov/pubmed/9566763/) | AA138- | AA1736- | AA1701- |
| | Notch2STS- | | 164 | 1757 | 1706 |
| | CD3zeta-CD28- | | | | |
| | Gal4VP64 | | | | |
| pRL_2105 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (ncbi.nlm.nih.gov/pubmed/9566763/) | AA138- | AA1736- | AA1701- |
| | Notch2STS- | | 164 | 1757 | 1706 |
| | CD3zeta- | | | | |
| | Gal4VP64- | | | | |
| | CD28 | | | | |
| pRL_2106 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (ncbi.nlm.nih.gov/pubmed/9566763/) | AA138- | AA1736- | AA1701- |
| | Notch2STS- | | 164 | 1757 | 1706 |
| | CD28-CD3zeta- | | | | |
| | Gal4VP64 | | | | |
| pRL_2107 | antiCD19scFv- | Uniprot P01732: AA1-21, FLAG | Uniprot | Uniprot | Uniprot |
| | CD8Hinge2- | TAG?9, FMC63 scFv sequence | P01732: | P46531: | Q04721: |
| | Notch1TMD- | (ncbi.nlm.nih.gov/pubmed/9566763/) | AA138- | AA1736- | AA1701- |
| | Notch2STS- | | 164 | 1757 | 1706 |
| | Gal4VP64- | | | | |
| | CD28-CD3zeta | | | | |
| pRL_2108 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (ncbi.nlm.nih.gov/pubmed/9566763/) | AA138- | AA1736- | AA1701- |
| | Notch2STS- | | 164 | 1757 | 1706 |
| | Gal4VP64- | | | | |
| | CD3zeta-CD28 | | | | |
| pRL_2144 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (https://www.ncbi.nlm.nih.gov/pubmed/9566763/) | AA138- | AA1736- | AA1701- |
| | Notch2STS- | | 164 | 1757 | 1706 |
| | noSTSCD28- | | | | |
| | CD3-Gal4VP64 | | | | |
| pRL_2146 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (https://www.ncbi.nlm.nih.gov/pubmed/9566763/) | AA138- | AA1736- | AA1701- |
| | Notch2STS- | | 164 | 1757 | 1706 |
| | truncCD28- | | | | |
| | CD3-Gal4VP64 | | | | |
| pRL_2205 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (https://www.ncbi.nlm.nih.gov/pubmed/9566763/) | AA138- | AA1736- | AA1701- |
| | Notch2STS- | | 164 | 1757 | 1706 |
| | CD28delTPRRP- | | | | |
| | CD3-Gal4VP64 | | | | |
| pRL_2206 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (https://www.ncbi.nlm.nih.gov/pubmed/9566763/) | AA138- | AA1736- | AA1701- |
| | Notch2STS- | | 164 | 1757 | 1706 |
| | truncCD28delTP | | | | |
| | RRP-CD3- | | | | |
| | Gal4VP64 | | | | |
| pRL_2207 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (https://www.ncbi.nlm.nih.gov/pubmed/9566763/) | AA138- | AA1736- | AA1701- |
| | Notch2STS- | | 164 | 1757 | 1706 |
| | fullytruncCD28- | | | | |
| | CD3-Gal4VP64 | | | | |

| Plasmid | Intracellular | Intracellular | Intracellular |
|---|---|---|---|
| ID | Domain 1 | Domain 2 | Domain 3 |
| pRL_2080 | Uniprot P10747: AA180- | Uniprot P04386: AA1-147, | Uniprot P20963-3: |
| | 220 | Uniprot P06492: AA437- | AA52-164 |
| | | 447 plus GS linker | |
| | | altogether repeated 4 times | |
| pRL_2081 | Uniprot P20963-3: AA52- | Uniprot P10747: AA180- | Uniprot P04386: |
| | 164 | 220 | AA1-147, Uniprot |
| | | | P06492: AA437-447 |
| | | | plus GS linker |
| | | | altogether repeated 4 |
| | | | times |
| pRL_2082 | Uniprot P20963-3: AA52- | Uniprot P04386: AA1-147, | Uniprot P10747: |
| | 164 | Uniprot P06492: AA437- | AA180-220 |
| | | 447 plus GS linker | |
| | | altogether repeated 4 times | |
| pRL_2083 | Uniprot P10747: AA180- | Uniprot P20963-3: AA52- | Uniprot P04386: |
| | 220 | 164 | AA1-147, Uniprot |
| | | | P06492: AA437-447 |
| | | | plus GS linker |
| | | | altogether repeated 4 |
| | | | times |
| pRL_2084 | Uniprot P04386: AA1-147, | Uniprot P10747: AA180- | Uniprot P20963-3: |
| | Uniprot P06492: AA437- | 220 | AA52-164 |
| | 447 plus GS linker | | |
| | altogether repeated 4 times | | |
| pRL_2085 | Uniprot P04386: AA1-147, | Uniprot P20963-3: AA52- | Uniprot P10747: |
| | Uniprot P06492: AA437- | 164 | AA180-220 |
| | 447 plus GS linker | | |
| | altogether repeated 4 times | | |
| pRL_2103 | Uniprot P10747: AA180- | Uniprot P04386: AA1-147, | Uniprot P20963-3: |
| | 220 | Uniprot P06492: AA437- | AA52-164 |
| | | 447 plus GS linker | |
| | | altogether repeated 4 times | |
| pRL_2104 | Uniprot P20963-3: AA52- | Uniprot P10747: AA180- | Uniprot P04386: |
| | 164 | 220 | AA1-147, Uniprot |
| | | | P06492: AA437-447 |
| | | | plus GS linker |
| | | | altogether repeated 4 |
| | | | times |
| pRL_2105 | Uniprot P20963-3: AA52- | Uniprot P04386: AA1-147, | Uniprot P10747: |
| | 164 | Uniprot P06492: AA437- | AA180-220 |
| | | 447 plus GS linker | |
| | | altogether repeated 4 times | |
| pRL_2106 | Uniprot P10747: AA180- | Uniprot P20963-3: AA52- | Uniprot P04386: |
| | 220 | 164 | AA1-147, Uniprot |
| | | | P06492: AA437-447 |
| | | | plus GS linker |
| | | | altogether repeated 4 |
| | | | times |
| pRL_2107 | Uniprot P04386: AA1-147, | Uniprot P10747: AA180- | Uniprot P20963-3: |
| | Uniprot P06492: AA437- | 220 | AA52-164 |
| | 447 plus GS linker | | |
| | altogether repeated 4 times | | |
| pRL_2108 | Uniprot P04386: AA1-147, | Uniprot P20963-3: AA52- | Uniprot P10747: |
| | Uniprot P06492: AA437- | 164 | AA180-220 |
| | 447 plus GS linker | | |
| | altogether repeated 4 times | | |
| pRL_2144 | Uniprot P10747: AA186- | Uniprot P20963-3: AA52- | Uniprot P04386: |
| | 220 | 164 | AA1-147, Uniprot |
| | | | P06492: AA437-447 |
| | | | plus GS linker |
| | | | altogether repeated 4 |
| | | | times |
| pRL_2146 | Uniprot P10747: AA189- | Uniprot P20963-3: AA52- | Uniprot P04386: |
| | 220 | 164 | AA1-147, Uniprot |
| | | | P06492: AA437-447 |
| | | | plus GS linker |
| | | | altogether repeated 4 |
| | | | times |
| pRL_2205 | Uniprot P10747: AA180- | Uniprot P20963-3: AA52- | Uniprot P04386: |
| | 194, AA200-220 | 164 | AA1-147, Uniprot |
| | | | P06492: AA437-447 |
| | | | plus GS linker |
| | | | altogether repeated 4 |
| | | | times |
| pRL_2206 | Uniprot P10747: AA189- | Uniprot P20963-3: AA52- | Uniprot P04386: |
| | 194, AA200-220 | 164 | AA1-147, Uniprot |
| | | | P06492: AA437-447 |
| | | | plus GS linker |
| | | | altogether repeated 4 |
| | | | times |
| pRL_2207 | Uniprot P10747: AA200- | Uniprot P20963-3: AA52- | Uniprot P04386: |
| | 220 | 164 | AA1-147, Uniprot |
| | | | P06492: AA437-447 |
| | | | plus GS linker |
| | | | altogether repeated 4 |
| | | | times |

In addition, the exemplary hybrid SynNotch CARs comprising 41BB and a CD28 costimulatory domain are described in Table 5 below.

TABLE 5
Receptor components description for 41BB/CD28 hybrid SynNotch CARs

| Plasmid | | | N- | | | | | |
|---|---|---|---|---|---|---|---|---|
| ID | Receptor description | ECD | JMD | TMD | STS | ICD 1 | ICD 2 | ICD 3 |
| pRL_2194 | 41BB Hybrid | CD8a | CD8a | Notch | Notch | truncated | CD3zeta | Gal4, |
| | SynNotch CAR | signal | Hinge | 1 | 2 | 41BB | | VP64 |
| | Configuration 4 with | peptide, | | | | with | | |
| | truncated 41BB | flag-tag, | | | | PYAP | | |
| | (removed Uniprot | anti-CD19 | | | | motif | | |
| | Q07011: AA214-230 | scFv | | | | from | | |
| | from pRL_2100) and | | | | | CD28 | | |
| | PYAP motif from | | | | | | | |
| | CD28 (Uniprot P10747: | | | | | | | |
| | AA208-211) | | | | | | | |
| pRL_2195 | 41BB Hybrid | CD8a | CD8a | Notch | Notch | truncated | CD3zeta | Gal4, |
| | SynNotch CAR | signal | Hinge | 1 | 2 | 41BB | | VP64 |
| | Configuration 4 with | peptide, | | | | with | | |
| | truncated 41BB | flag-tag, | | | | YMNM | | |
| | (removed Uniprot | anti-CD19 | | | | (N | | |
| | Q07011: AA214-230 | scFv | | | | mutated | | |
| | from pRL_2100) and | | | | | to F) | | |
| | YMNM motif from | | | | | motif | | |
| | CD28 (Uniprot P10747: | | | | | from | | |
| | AA191-194 with | | | | | CD28 | | |
| | AA193 Mutated from | | | | | | | |
| | N to F) | | | | | | | |
| pRL_2196 | 41BB Hybrid | CD8a | CD8a | Notch | Notch | truncated | CD3zeta | Gal4, |
| | SynNotch CAR | signal | Hinge | 1 | 2 | 41BB | | VP64 |
| | Configuration 4 with | peptide, | | | | with | | |
| | truncated 41BB | flag-tag, | | | | YMNMTPRRP | | |
| | (removed Uniprot | anti-CD19 | | | | (N | | |
| | Q07011: AA214-230 | scFv | | | | mutated | | |
| | from pRL_2100) and | | | | | to F) | | |
| | YMNMTPRRP motif | | | | | motif | | |
| | from CD28 (Uniprot | | | | | from | | |
| | P10747: AA191-199 | | | | | CD28 | | |
| | with AA193 Mutated | | | | | | | |
| | from N to F) | | | | | | | |
| pRL_2197 | 41BB Hybrid | CD8a | CD8a | Notch | Notch | truncated | CD3zeta | Gal4, |
| | SynNotch CAR | signal | Hinge | 1 | 2 | 41BB | | VP64 |
| | Configuration 4 with | peptide, | | | | with | | |
| | truncated 41BB | flag-tag, | | | | AAYRS | | |
| | (removed Uniprot | anti-CD19 | | | | motif | | |
| | Q07011: AA214-230 | scFv | | | | from | | |
| | from pRL_2100) and | | | | | CD28 | | |
| | AAYRS motif from | | | | | | | |
| | CD28 (Uniprot P10747: | | | | | | | |
| | AA216-220) | | | | | | | |
| pRL_2233 | 41BB Hybrid | CD8a | CD8a | Notch | Notch | truncated | CD3zeta | Gal4, |
| | SynNotch CAR | signal | Hinge | 1 | 2 | 41BB | | VP64 |
| | Configuration 4 with | peptide, | | | | with | | |
| | truncated 41BB | flag-tag, | | | | PYAP | | |
| | (removed Uniprot | anti- | | | | motif | | |
| | Q07011: AA214-230 | BCMA | | | | from | | |
| | from pRL_2100) and | scFv | | | | CD28 | | |
| | PYAP motif from | | | | | | | |
| | CD28 (Uniprot P10747: | | | | | | | |
| | AA208-211) | | | | | | | |
| pRL_2235 | 41BB Hybrid | CD8a | CD8a | Notch | Notch | truncated | CD3zeta | Gal4, |
| | SynNotch CAR | signal | Hinge | 1 | 2 | 41BB | | VP64 |
| | Configuration 4 with | peptide, | | | | with | | |
| | truncated 41BB | flag-tag, | | | | PYAP | | |
| | (removed Uniprot | anti- | | | | motif | | |
| | Q07011: AA214-230 | ALPPL2 | | | | from | | |
| | from pRL_2100) and | scFv | | | | CD28 | | |
| | PYAP motif from | | | | | | | |
| | CD28 (Uniprot P10747: | | | | | | | |
| | AA208-211) | | | | | | | |

Additionally, Table 6 below provides sequence references for receptor components for 41BB/CD28 hybrid SynNotch CARs.

TABLE 6
Sequence references for 41BB/CD28 hybrid SynNotch CARs.

| Plasmid | | | N- | | ICD |
|---|---|---|---|---|---|
| ID | Receptor | ECD | JMD | TMD | (STS) |
| pRL_2194 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (https://www.ncbi.nlm.nih.gov/pubmed/9566763/) | AA138- | AA1736- | AA1701- |
| | Notch2STS- | | 164 | 1757 | 1706 |
| | trunc41BB- | | | | |
| | PYAP-CD3z- | | | | |
| | Gal4VP64 | | | | |
| pRL_2195 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (https://www.ncbi.nlm.nih.gov/pubmed/9566763/) | AA138- | AA1736- | AA1701- |
| | Notch2STS- | | 164 | 1757 | 1706 |
| | trunc41BB- | | | | |
| | YMFM- | | | | |
| | CD3z- | | | | |
| | Gal4VP64 | | | | |
| pRL_2196 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (https://www.ncbi.nlm.nih.gov/pubmed/9566763/) | AA138- | AA1736- | AA1701- |
| | Notch2STS- | | 164 | 1757 | 1706 |
| | trunc41BB- | | | | |
| | YMFMTPRRP- | | | | |
| | CD3z- | | | | |
| | Gal4VP64 | | | | |
| pRL_2197 | antiCD19scFv- | Uniprot P01732: AA1-21, FLAG | Uniprot | Uniprot | Uniprot |
| | CD8Hinge2- | TAG?, FMC63 scFv sequence | P01732: | P46531: | Q04721: |
| | Notch1TMD- | (https://www.ncbi.nlm.nih.gov/pubmed/9566763/) | AA138- | AA1736- | AA1701- |
| | Notch2STS- | | 164 | 1757 | 1706 |
| | trunc41BB- | | | | |
| | AAYRS- | | | | |
| | CD3z- | | | | |
| | Gal4VP64 | | | | |
| pRL_2233 | antiBCMAscFv- | Uniprot P01732: AA1-21, FLAG | Uniprot | Uniprot | Uniprot |
| | CD8Hinge2- | TAG?, BCMA-50 scFv sequence | P01732: | P46531: | Q04721: |
| | Notch1TMD- | (US Patent No. 2017/01834.18 A1) | AA138- | AA1736- | AA1701- |
| | Notch2STS- | | 164 | 1757 | 1706 |
| | trunc41BB- | | | | |
| | PYAP-CD3z- | | | | |
| | Gal4VP64 | | | | |
| pRL_2235 | antiALPPL2scFv- | Uniprot P01732: AA1-21, FLAG | Uniprot | Uniprot | Uniprot |
| | CD8Hinge2- | TAG?, M25 FYIA scFv sequence | P01732: | P46531: | Q04721: |
| | Notch1TMD- | (https://pubmed.ncbi.nlm.nih.gov/32868383/) | AA138- | AA1736- | AA1701- |
| | Notch2STS- | | 164 | 1757 | 1706 |
| | trunc41BB- | | | | |
| | PYAP-CD3z- | | | | |
| | Gal4VP64 | | | | |

| Plasmid | Intracellular | Intracellular | Intracellular |
|---|---|---|---|
| ID | Domain 1 | Domain 2 | Domain 3 |
| pRL_2194 | Uniprot Q07011: AA231- | Uniprot P20963-3: AA52- | Uniprot P04386: |
| | 255, Uniprot P10747: | 164 (WE USE UPENN | AA1-147, Uniprot |
| | AA208-211 | CD3ZETA) | P06492: AA437-447 |
| | | | plus GS linker |
| | | | altogether repeated 4 |
| | | | times |
| pRL_2195 | Uniprot Q07011: AA231- | Uniprot P20963-3: AA52- | Uniprot P04386: |
| | 255, Uniprot P10747: | 164 (WE USE UPENN | AA1-147, Uniprot |
| | AA191-194 with AA193 | CD3ZETA) | P06492: AA437-447 |
| | Mutated from N to F | | plus GS linker |
| | | | altogether repeated 4 |
| | | | times |
| pRL_2196 | Uniprot Q07011: AA231- | Uniprot P20963-3: AA52- | Uniprot P04386: |
| | 255, Uniprot P10747: | 164 | AA1-147, Uniprot |
| | AA191-199 with AA193 | | P06492: AA437-447 |
| | Mutated from N to F | | plus GS linker |
| | | | altogether repeated 4 |
| | | | times |
| pRL_2197 | Uniprot Q07011: AA231- | Uniprot P20963-3: AA52- | Uniprot P04386: |
| | 255, Uniprot P10747: | 164 | AA1-147, Uniprot |
| | AA216-220 | | P06492: AA437-447 |
| | | | plus GS linker |
| | | | altogether repeated 4 |
| | | | times |
| pRL_2233 | Uniprot Q07011: AA231- | Uniprot P20963-3: AA52- | Uniprot P04386: |
| | 255, Uniprot P10747: | 164 | AA1-147, Uniprot |
| | AA208-211 | | P06492: AA437-447 |
| | | | plus GS linker |
| | | | altogether repeated 4 |
| | | | times |
| pRL_2235 | Uniprot Q07011: AA231- | Uniprot P20963-3: AA52- | Uniprot P04386: |
| | 255, Uniprot P10747: | 164 | AA1-147, Uniprot |
| | AA208-211 | | P06492: AA437-447 |
| | | | plus GS linker |
| | | | altogether repeated 4 |
| | | | times |

### Example 2. Methods

This Example describes the additional methods used in the present disclosure.

### Primary Human T Cell Isolation and Culture

Primary CD4+ and CD8+ T cells were isolated from anonymous donor blood after apheresis by negative selection (STEMCELL Technologies #15062 & 15063). Blood was obtained from Blood Centers of the Pacific (San Francisco, CA) as approved by the University Institutional Review Board. T cells were cryopreserved in RPMI-1640 (UCSF cell culture core) with 20% human AB serum (Valley Biomedical Inc., #HP1022) and 10% DMSO. After thawing, T cells were cultured in human T cell medium consisting of X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments. In vivo experiments were completed with bulk CD3+ cells isolated in a similar manner.

### Lentiviral Transduction of Human T Cells

Pantropic VSV-G pseudotyped lentivirus was produced via transfection of Lenti-X 293T cells (Clontech #11131D) with a pHR′SIN:CSW transgene expression vector and the viral packaging plasmids pCMVdR8.91 and pMD2.G using Mirus TransIT-Lenti (Mirus #MIR 6606). Primary T cells were thawed the same day, and after 24 hours in culture, were stimulated with Human T-Activator CD3/CD28 Dynabeads (Life Technologies #11131D) at a 1:3 cell:bead ratio. At 48 hours, viral supernatant was harvested and the primary T cells were exposed to the virus for 24 hours. At day 5 post T cell stimulation, the Dynabeads were removed, T cells were sorted, and the T cells expanded until day 10-14 when they were rested and could be used in vitro or in vivo assays. T cells were sorted for assays with a Beckton Dickinson (BD) FACs ARIA II.

### Cancer Cell Lines

The cancer cell lines used were K562 myelogenous leukemia cells (ATCC #CCL-243), A549 lung epithelial carcinoma cells (ATCC #CCL-18) and M28 human epithial type mesothelioma cells. K562s, A549s and M28s were lentivirally transduced to stably express human CD19. CD19 levels were determined by staining the cells with α-CD19 APC (Biolegend #302212) or BV421 (Biolegend #302234). A549s were additionally transduced to express the nuclear stain mkate2. All cell lines were sorted for expression of the transgenes.

### Circuit Induction Assay

To assess circuit induction, primary human T cells were co-transduced with the hybrid SynNotch CAR and a response element containing a UAS promoted BFP gene, transduced cells were then co-cultured with K562s, either with or without CD19, at a 1:1 ratio for 24-72 hours. Co-cultured cells were then centrifuged, washed twice with flow buffer (PBS+2% FBS), and resuspended in flow buffer with diluted DRAQ7 to assess viability. Washed cells were immediately analyzed on a flow cytometer to assess expression of BFP.

### In Vitro Activation Assay

The expression of surface activation markers was used as a measure of short-term activation by either CAR or hybrid SynNotch CAR Signaling. To determine the activation, transduced cells were co-cultured with K562s, either with or without CD19 expression, at a 1:1 ratio for 24-72 hours. Co-cultured cells were then centrifuged, washed twice with flow buffer (PBS+2% FBS), and stained in 50 uL of a master mix of antibodies targeting surface activation markers CD69, PD-1, CD25 and CD39. Cells were washed twice after stain with flow buffer and resuspended in flow buffer with diluted DRAQ7 to assess viability. Stained cells were immediately analyzed on a flow cytometer to assess expression of activation markers.

In vitro Cytokine Secretion Assay

To assess cytokine secretion, transduced primary human T cells were co-cultured with K562s, either with or without CD19 expression, at a 1:1 ratio for 18-24 hours (overnight), 48 hours (short term) or 96 hours (long term). Overnight co-cultures included Brefeldin A (eBioscience #00-4506-51) and Monesin (VWR #420701-BL) to stop secretion of cytokines. For both short and long-term co-cultures, Brefeldin A and Monesin and a second bolus of K562s and was added to the co-culture and incubated for an additional 6 hours before beginning staining. Co-cultured cells were washed twice with PBS and stained in 50 uL of Fixable NEAR IR (Invitrogen #L34975) for 20 minutes at room temperature in the dark. 50 uL of a master mix containing fluorescently tagged anti-CD4 or anti-CD8 antibodies was then added to the cells and incubated for 20 minutes at room temperature in the dark. Stained cells were then washed twice with flow buffer (PBS+2% FBS). Stained cells were then resuspended in 100 uL of IC Fix Buffer (eBioscience #00-8222-49) and incubated for 45 minutes at 4 C in the dark. Fixed cells were then washed twice with 1× Permeabilization Buffer (eBioscience #00-8333-56). An intracellular cytokine staining master mix was made of fluorescently tagged antibodies targeting intracellular cytokines TNFα, IL-2, IFNy and GranzymeB diluted in 1× Permeabilization Buffer. Washed cells were stained in 50 uL of this master mix for 30 minutes at 4 C in the dark. Stained cells were washed twice with 1× Permeabilization Buffer and resuspended in 100 uL of flow buffer. Stained cells were immediately analyzed on a flow cytometer.

### In Vitro Incucyte Target Killing Assay

CD19+A549 cells expressing mkate2 were seeded in a flat bottom 96 well plate and incubated overnight to allow adherence. Transduced primary human T cells were centrifuged and resuspended in Jurkat media+30 U/mL IL-2; Jurkat media (RPMI-1640 medium+10% FBS+1% PenStrep+1× Glutamax) as RPMI has less fluorescence than media based on X-VIVO-15. Media was removed from the adherent A549 cells, and transduced human T cells were added to cultures at a 1:1 ratio. Images were taken every 2 hours using the Incucyte software over the course of the experiments (see relevant figures for imaging total assay times, which varied between conditions).

### In Vitro Proliferation Assay

Transduced human T cells were taken from culture and washed into PBS with diluted CellTrace Far Red (CTFR) (Invitrogen #C34564). Cells were stained for 20 minutes at 37° C. in the dark, then 5× the staining volume of culture media with protein was added, and cells were incubated for an additional 5 minutes at 37° C. in the dark. Stained cells were centrifuged, washed into human T cell media. K562 cells with and without CD19 expression were washed into human T cell media and added to CTFR stained T cells at a 1:1 ratio. Co-cultures were incubated for 5 days, with a media change occurring halfway through incubation. Co-cultures were then centrifuged, washed twice with flow buffer (PBS+2% FBS), and stained in 50 μL of master mix containing fluorescently tagged anti-CD8 antibodies. Cells were washed twice after stain with flow buffer and resuspended in flow buffer with diluted DRAQ7 to assess viability. Stained cells were immediately analyzed on a flow cytometer to assess dilution of CTFR dye.

### In Vivo Nalm6 Study

NOD.Cg-Prkdcscid Il2rgtm1Wjl/SzJ (NSG) (UCSF LARC Breeding Core) mice were dosed with 0.5×106 Luciferase expressing Nalm 6 cells via tail vein injection. 4 days post tumor injection, hybrid SynNotch CAR or CAR transduced T cells were dosed to tumor bearing animals via retro-orbital injection (see figures details for the number of T cells dosed per experiment). Bioluminescence imaging was performed using an IVIS Spectrum In Vivo Imaging system at regular time points to assess tumor burden. Animals were dosed with 200 μL of 15 mg/mL Luciferin via IP injection, and allowed to ambulate for 12-20 minutes prior to capturing prone and supine images. Image capture time was adjusted based on bioluminescence intensity, and average radiance [p/s/cm²/sr] was used as a measurement of tumor burden. Throughout experiment animal drinking water was supplemented with Clavomox (Zoetis #55-101) to prevent bacterial infections.

### In Vivo M28 Study

NOD.Cg-Prkdcscid Il2rgtm1Wjl/SzJ (NSG) mice were dosed with 4×106 CD19 ligand expressing M28 cells via subcutaneous injection. 7 days post tumor injection, 3-6×106 Hybrid SynNotch CAR or CAR transduced T cells were dosed to tumor bearing animals via retro-orbital injection. Tumors were measured with calipers twice weekly, and tumor volume was calculated using the following formula: (length×width2)/2. Throughout experiment animal drinking water was supplemented with Clavomox to prevent bacterial infections.

### Example 3: Hybrid SynNotch Car Expression and Circuit Induction

This Example demonstrates that the exemplary hybrid SynNotch CARs provided herein are able to induce expression of the transcriptional circuit and activate human T-cells.

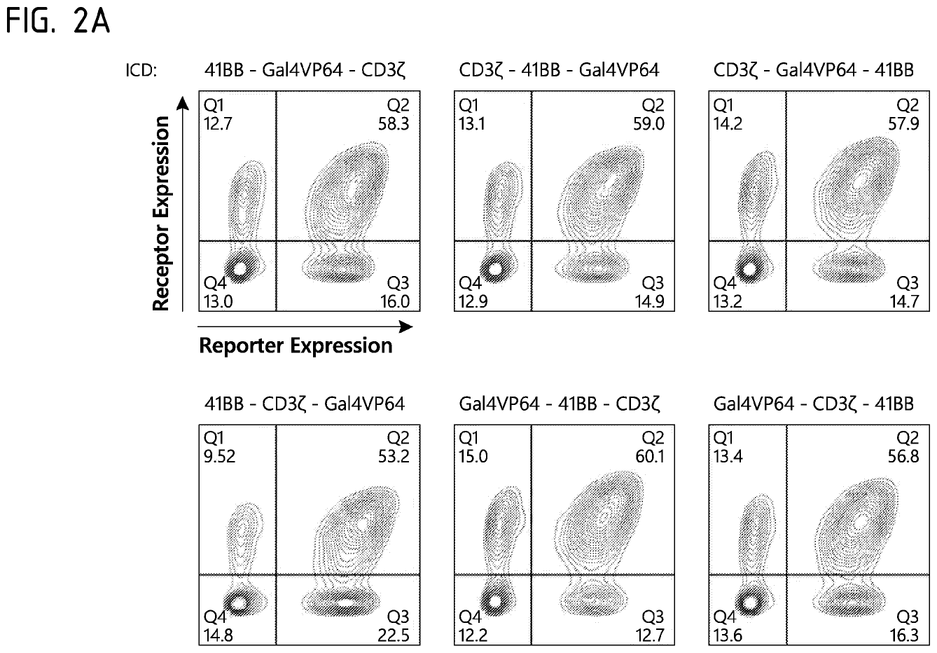

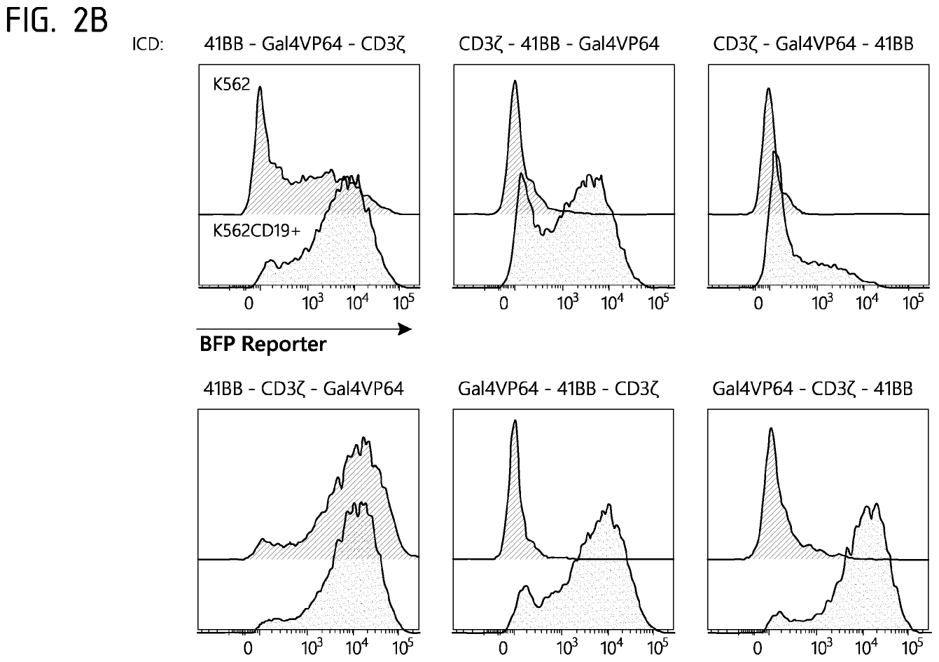

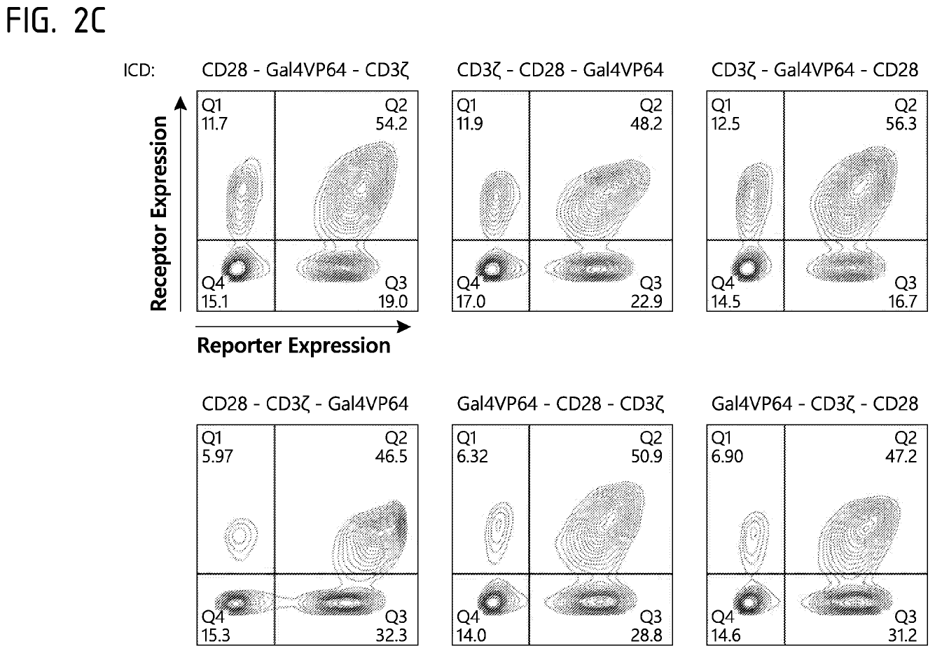

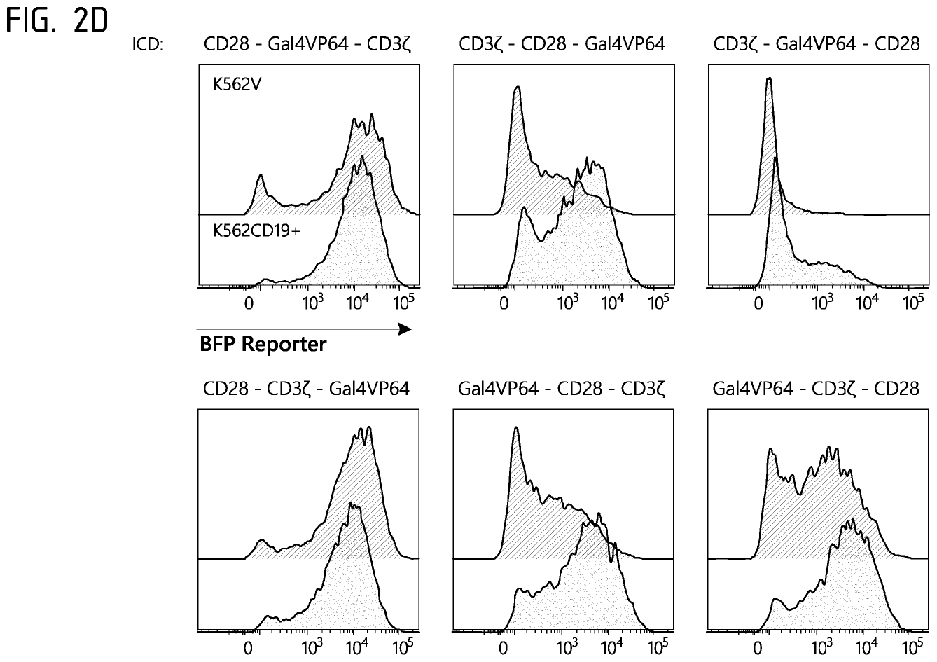

Briefly, primary human T-cells were activated with anti-CD3/anti-CD28 Dynabeads (Gibco) and transduced with two lentiviral constructs expressing either a receptor or a transcriptional reporter construct. Hybrid SynNotch CAR with 4-1BB costimulatory domains (FIG. 2A) or CD28 costimulatory domains (FIG. 2C) were sorted on Day 5 post initial T-cell stimulation, purifying the receptor and reporter dual positive population. To assess circuit induction, on Day 14 post initial T cell stimulation, T-cells expressing anti-CD19 receptors with 4-1BB costimulatory domains (FIG. 2B) or CD28 costimulatory domains (FIG. 2D) and the BFP reporter were co-cultured with K562 cells (top), or CD19+ K562 cells (bottom) for 48 hours. Transcriptional activation of the inducible BFP reporter gene was subsequently measured using a Fortessa X-50 (BD).

As demonstrated, some intracellular configurations of the Hybrid SynNotch CARs with 4-1BB or CD28 costimulatory domains exhibit antigen independent induction of the inducible transcriptional BFP reporter element (e.g., For 4-1BB Configuration 1 and 4, for CD28 configurations 1, 2, 4, 5, and 6). However, other intracellular configurations of the Hybrid SynNotch CARs exhibit antigen specific induction of the BFP reporter element, expressing BFP only when in the presence of ligand (e.g., For 4-1BB configurations 2, 3, 5, and 6, for CD28 configuration 3). This data set demonstrates the ability of specific configurations of Hybrid SynNotch CAR circuits that incorporate either the 4-1BB or CD28 co-stimulatory domains to induce transcription in an antigen specific manner. Additionally, this data set indicates that the spatial configuration of the intracellular domains influences receptor behavior in both the presence and absence of ligand.

### Example 4: Hybrid SynNotch Car Activation Marker Expression

This Example shows the expression of the activation markers of the T cells transduced with the exemplary hybrid SynNotch CARs.

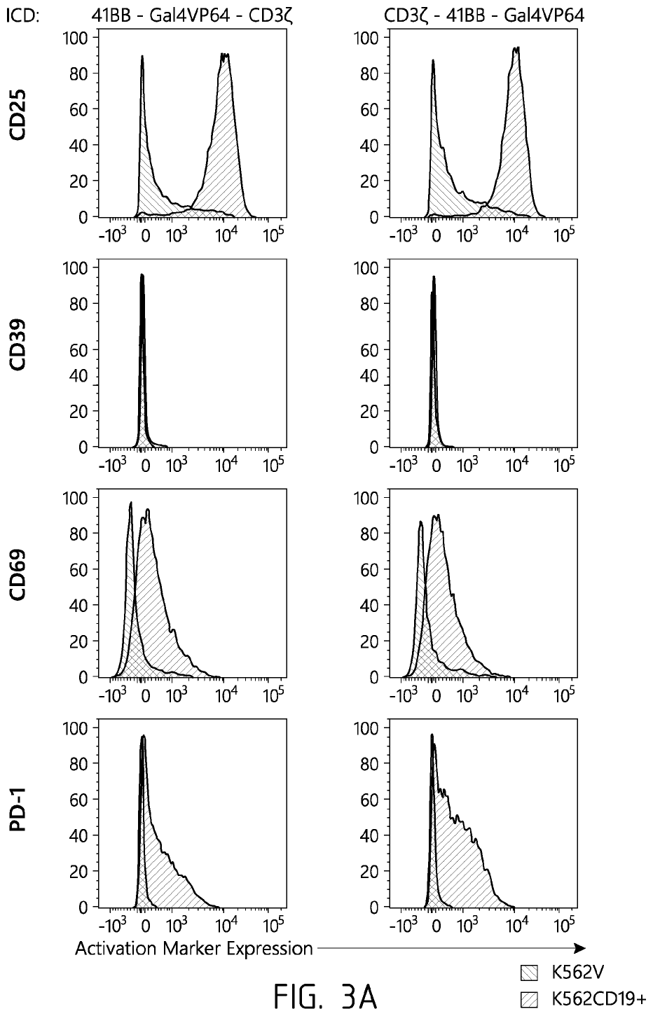

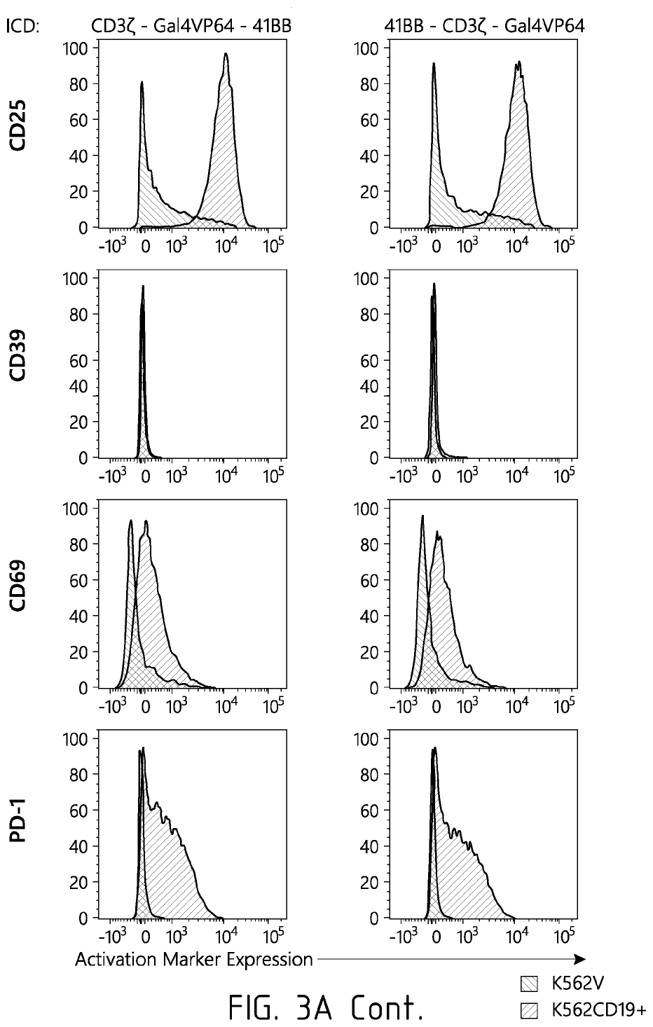

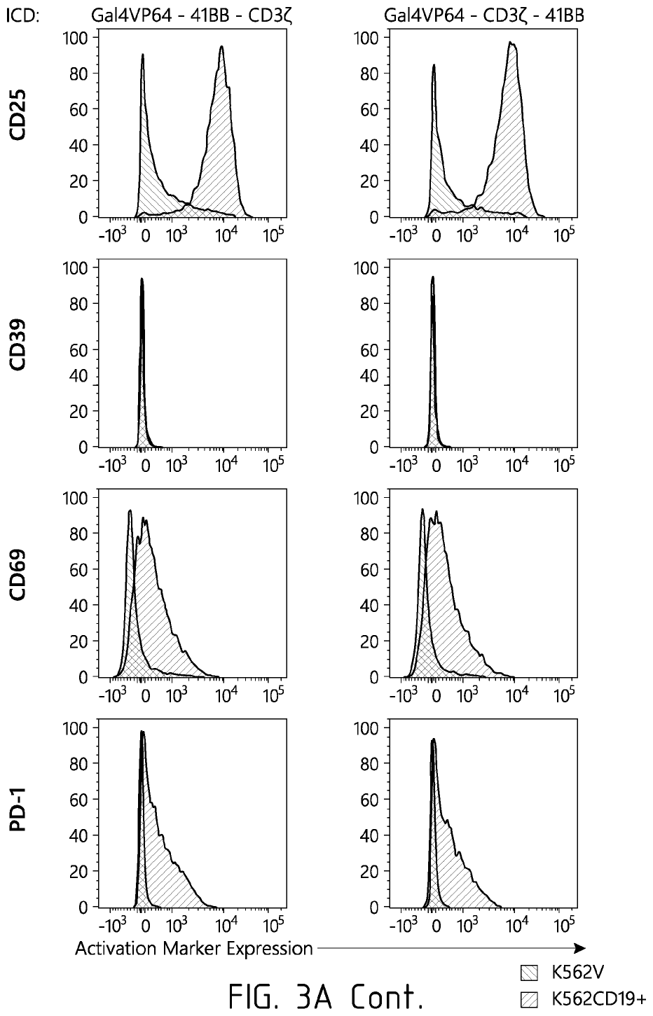

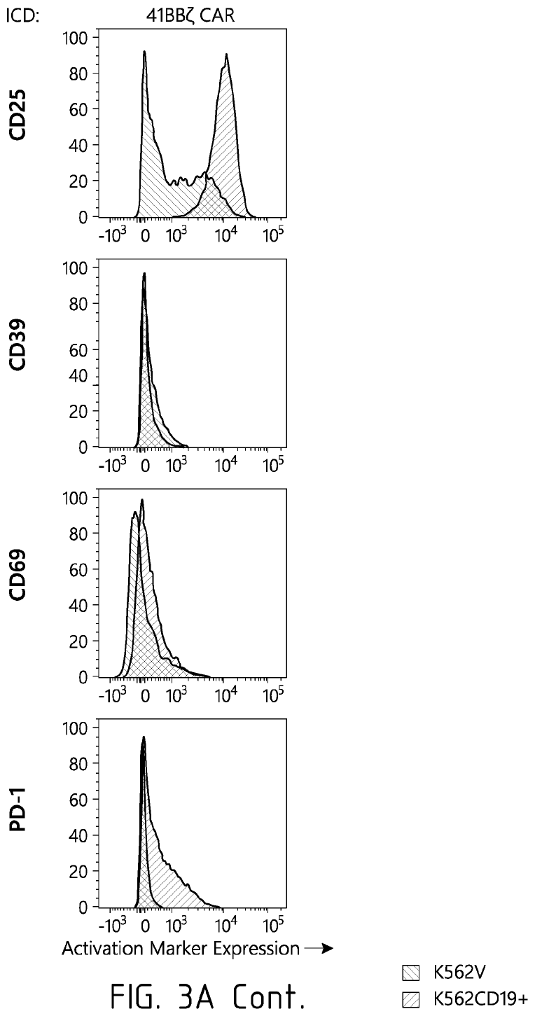

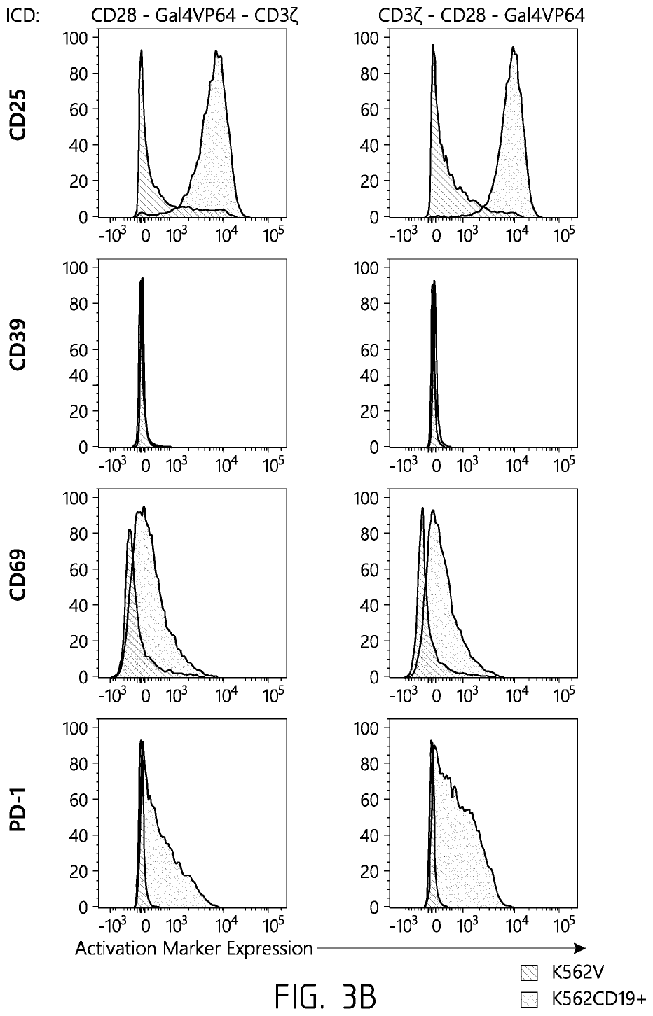

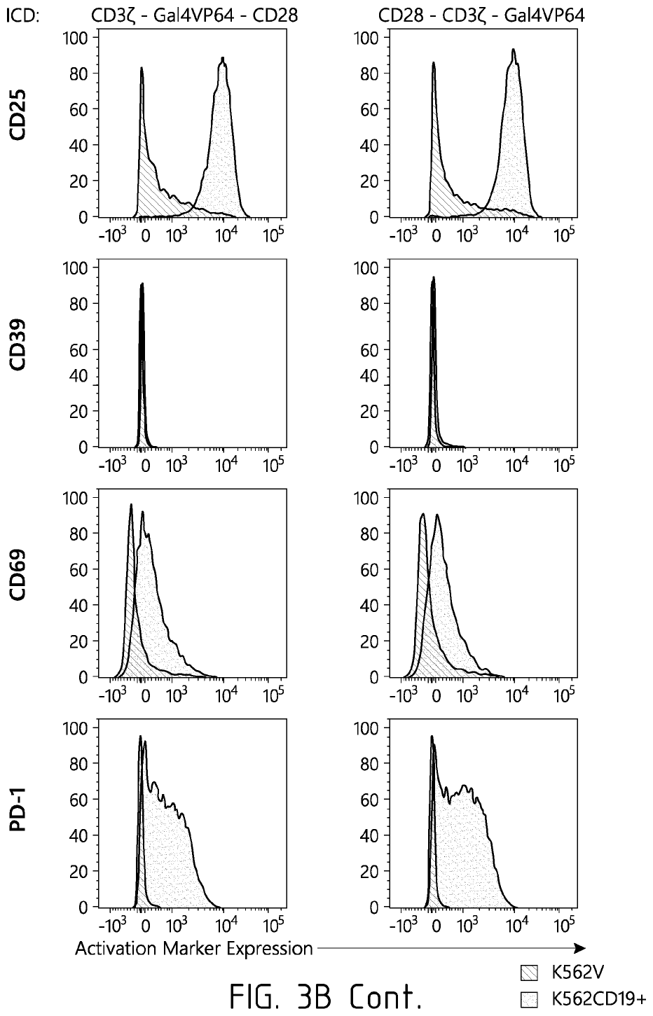

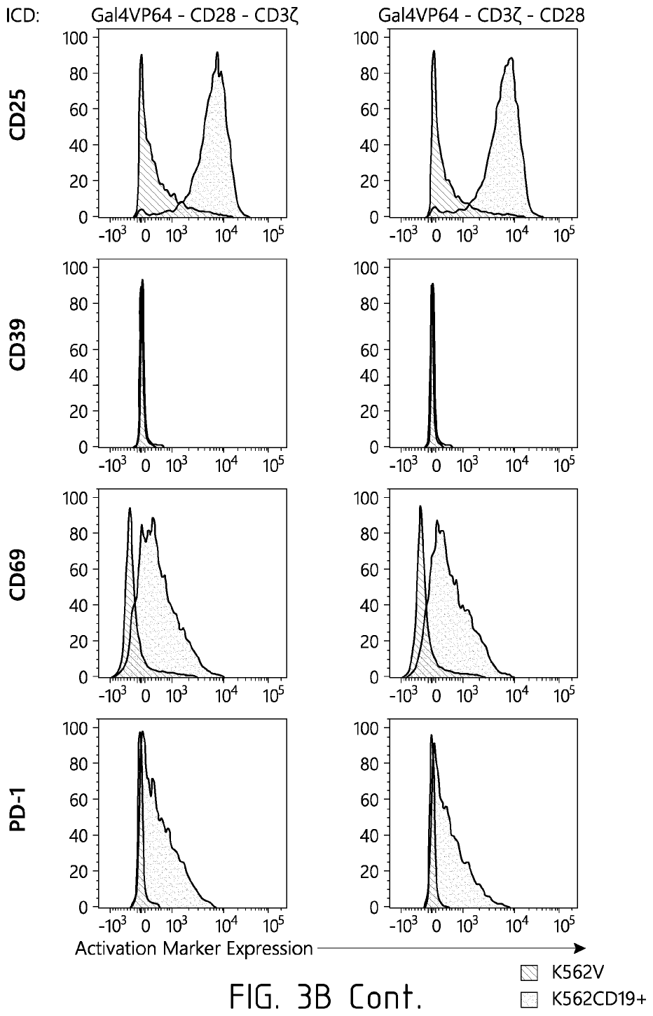

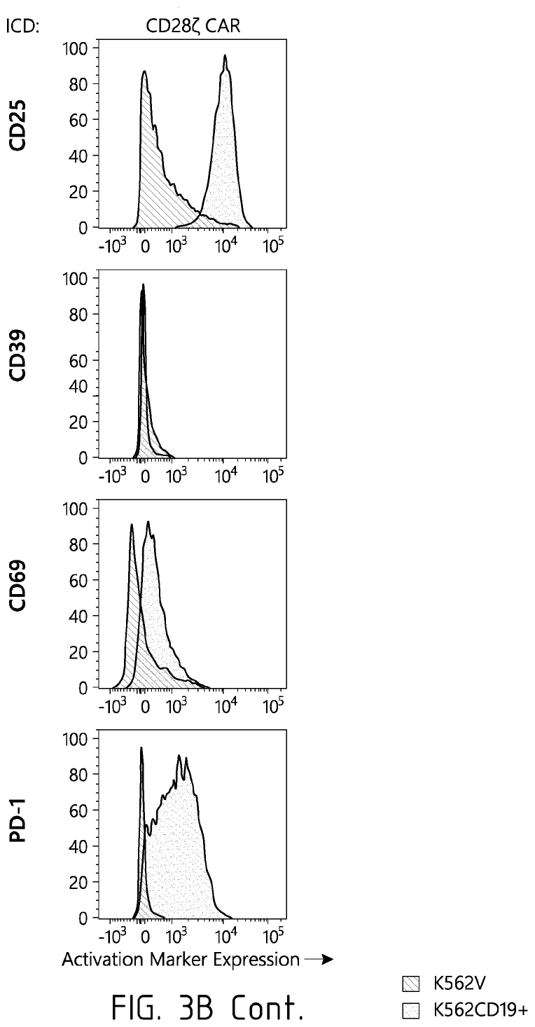

T-cells expressing anti-CD19 receptors with 4-1BB costimulatory domains (FIG. 3A) or CD28 costimulatory domains (FIG. 3B) and the BFP reporter were produced as described above. Transduced cells were co-cultured with K562 cells (gray), or CD19+ K562 cells (light gray) for 48 hours. Expression of activation markers CD25, CD39, CD69 and PD-1 were subsequently measured using a Fortessa X-50 (BD). As demonstrated, the Hybrid SynNotch CAR T cells express activation markers at a similar mean fluorescence intensity and overall percentage as compared to the CAR control that employs the same co-stimulatory domain. Additionally, the Hybrid SynNotch CAR T cells only express activation markers when in the presence of ligand. Together, this data set indicates that, upon engagement with antigen, Hybrid SynNotch CARs functionally induce signaling through their intracellular signaling domains (4-1BB or CD28, and CD3zeta), leading to expression of activation markers.

### Example 5: Hybrid SynNotch Car Proliferation

This Example shows the proliferation of the T cells transduced with the exemplary hybrid SynNotch CARs.

T-cells expressing anti-CD19 receptors with 4-1BB costimulatory domains or CD28 costimulatory domains and the BFP reporter were produced as described above. Transduced cells were stained with Cell Trace Far Red (CTFR), then co-cultured with K562 cells (gray), or CD19+ K562 cells (blue or red) for 5 days. Dilution of CTFR dye was subsequently measured using a Fortessa X-50 (BD).

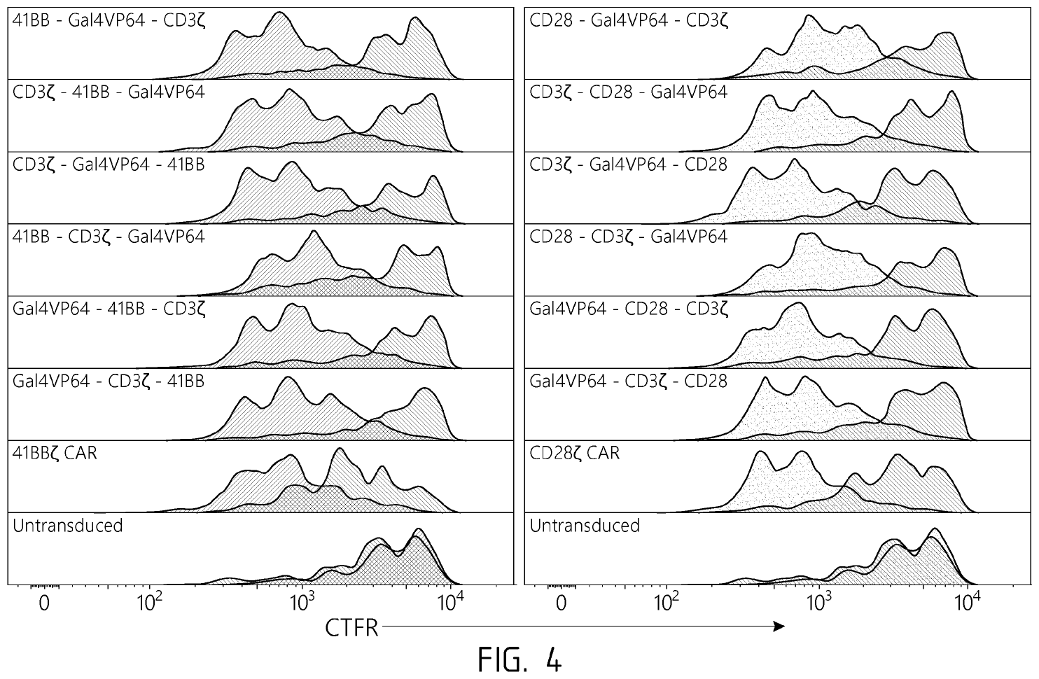

As shown in FIG. 4, when engaged with ligand, the Hybrid SynNotch CARs induce T cell proliferation at a rate similar to the CAR alone. Additionally, the Hybrid SynNotch CAR T cells specifically proliferate extensively when antigen is present, indicating that the proliferative response of the Hybrid SynNotch CAR T cells is antigen specific. Altogether, this data set indicates that, upon engagement with antigen, Hybrid SynNotch CARs functionally induce short term signaling cascades through their intracellular signaling domains (4-1BB or CD28, and CD3zeta), leading to proliferation of the T cells.

### Example 6: Hybrid SynNotch Car Cytokine Secretion

This Example shows the cytokine secretion by the T cells transduced with the exemplary hybrid SynNotch CARs.

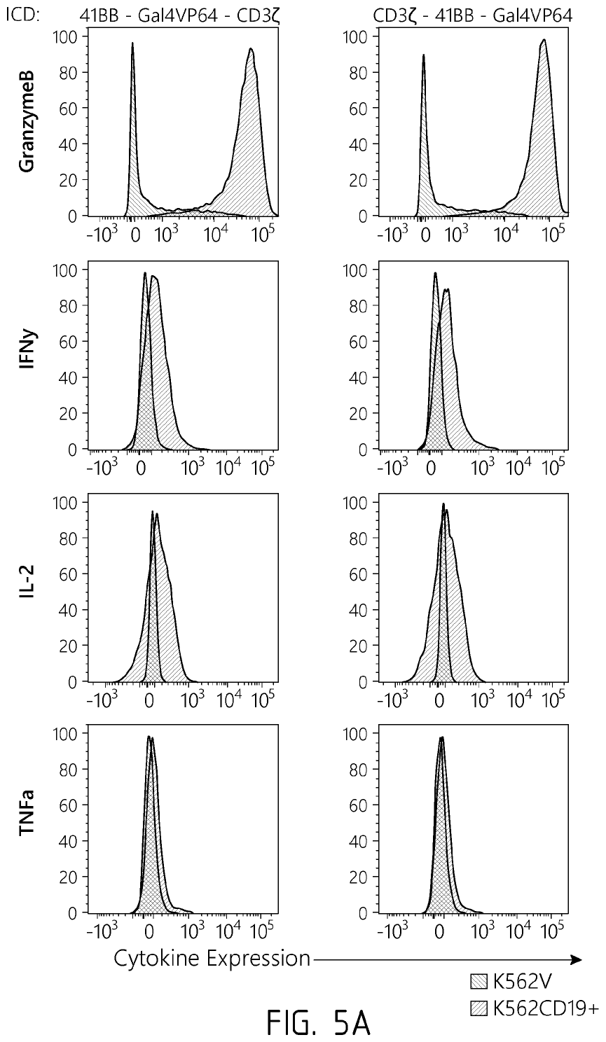

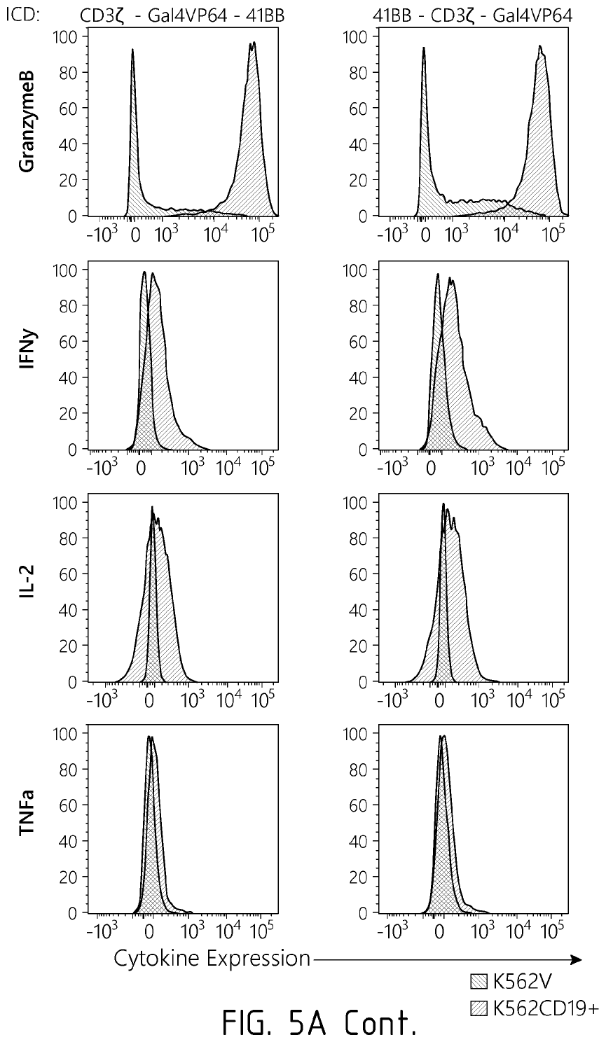

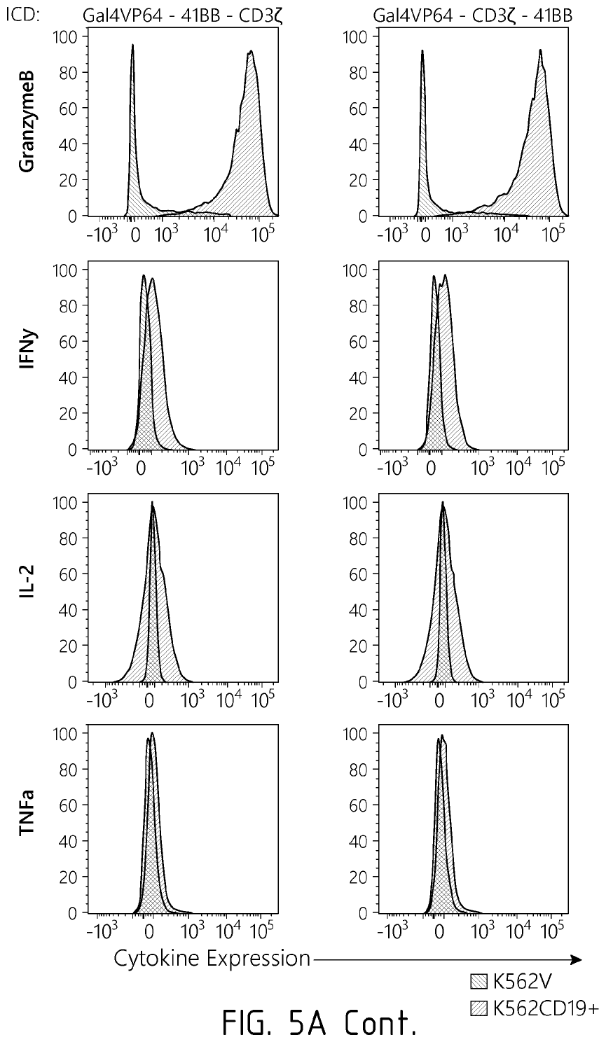

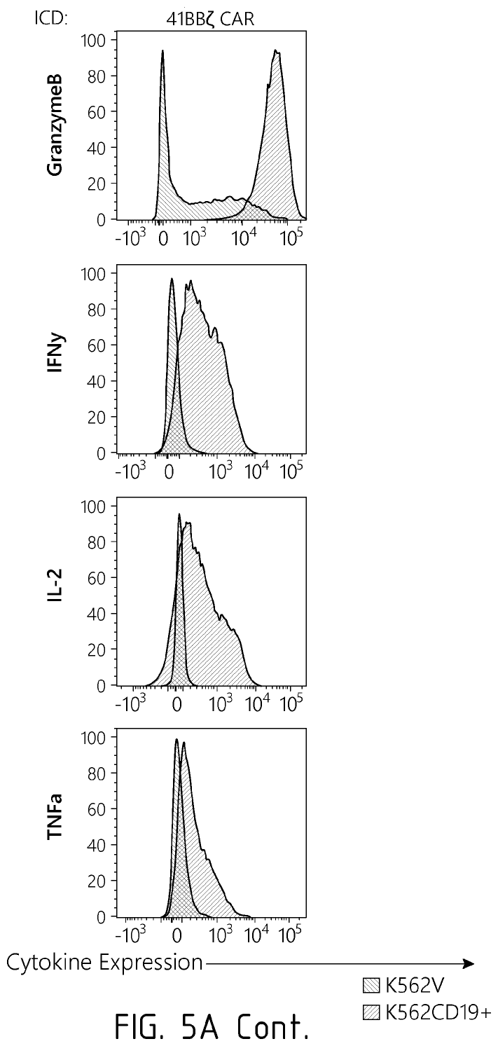

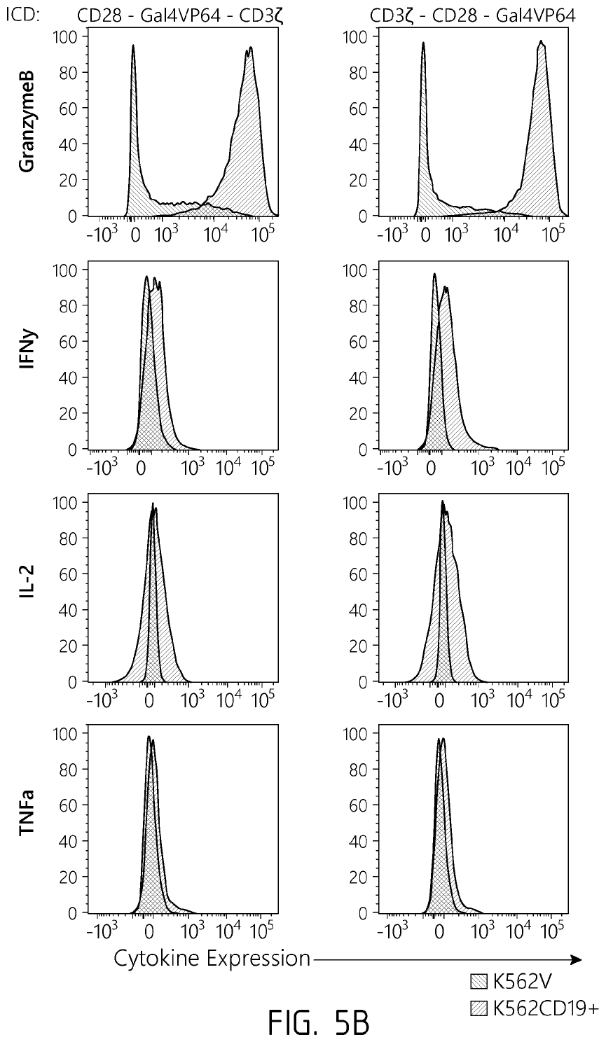

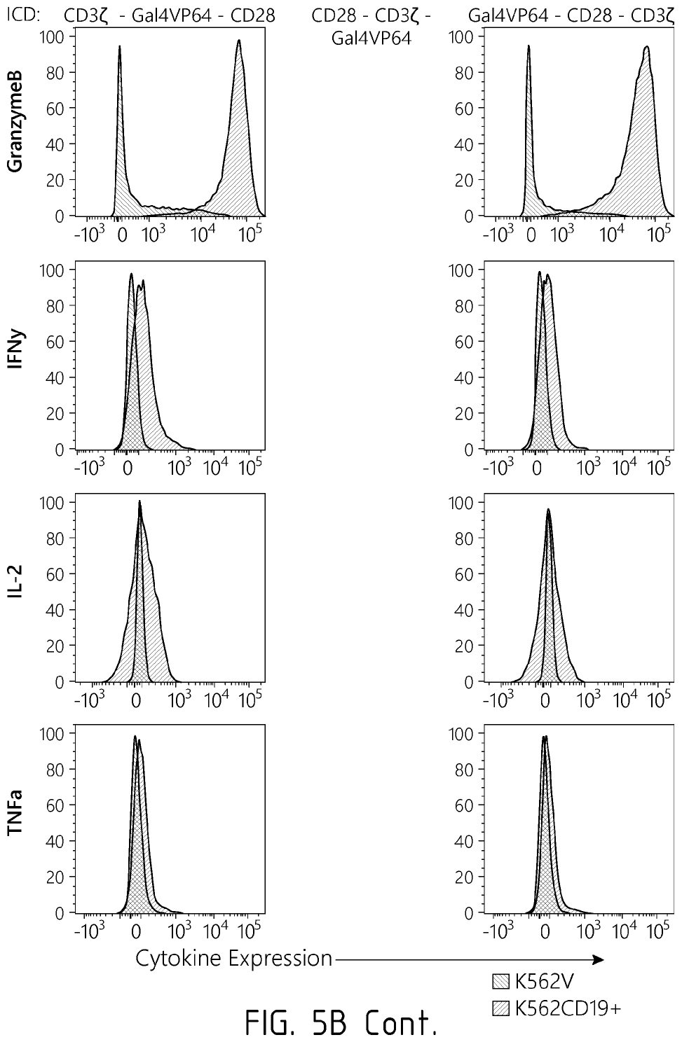

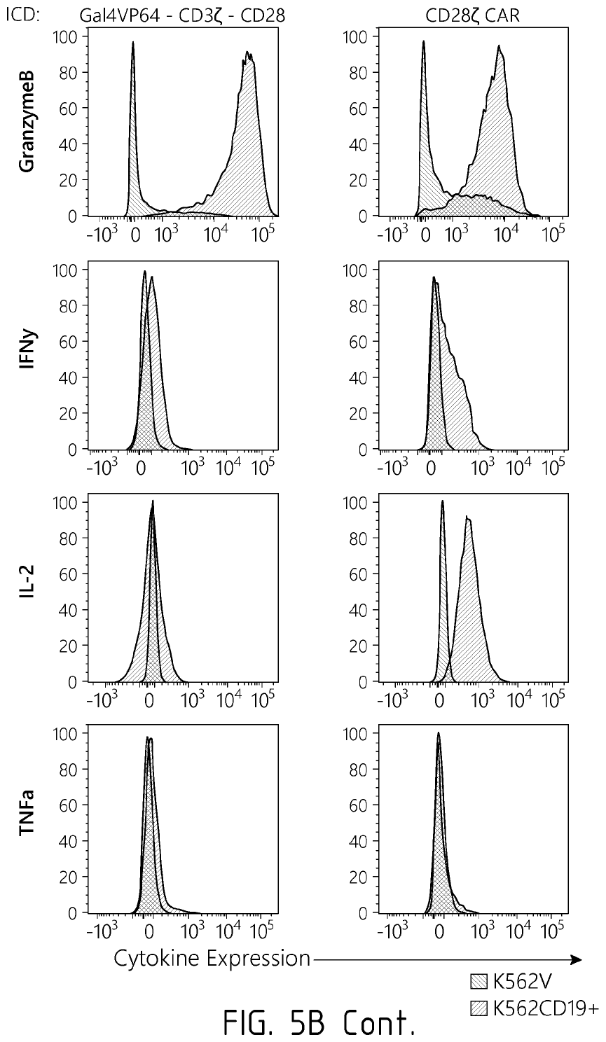

T-cells expressing anti-CD19 receptors with 4-1BB costimulatory domains (FIG. 5A) or CD28 costimulatory domains (FIG. 5B) and the BFP reporter were produced as above. Transduced cells were co-cultured with K562 cells (gray), or CD19+ K562 cells (blue or red). After 48 hours, Brefeldin A, Monesin and a second bolus of K562 cells (either with or without CD19+ expression) was added to the co-cultures. Co-cultures were incubated for an additional 6 hours, then transduced cells were assessed using a Fortessa X50 (BD) for intracellular expression of the cytokines Granzyme B, IFNy, IL-2, and TNFα. As demonstrated, the Hybrid SynNotch CARs induce lower expression of cytokines (except GranzymeB) as compared to the CARs alone. This data set suggests that the signaling induced by the costimulatory domains and CD3zeta domain of the Hybrid SynNotch CARs differs in type or mechanism, strength, intensity, or length of time to the CARs.

### Example 7: Hybrid SynNotch Car Target Killing

This Example shows the cell killing activity of the exemplary hybrid SynNotch CARs.

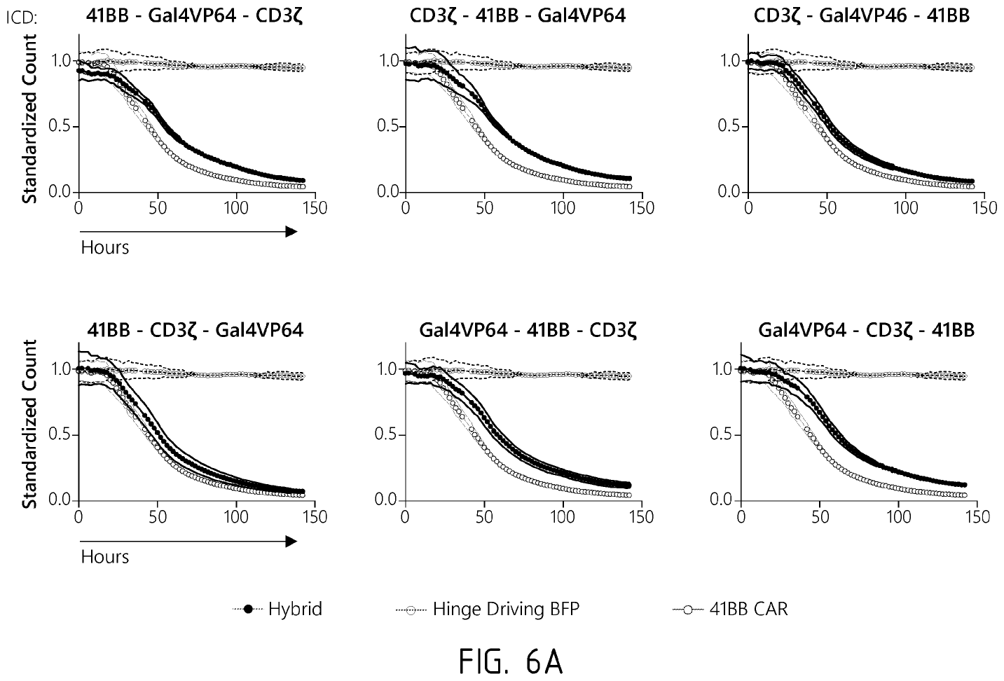

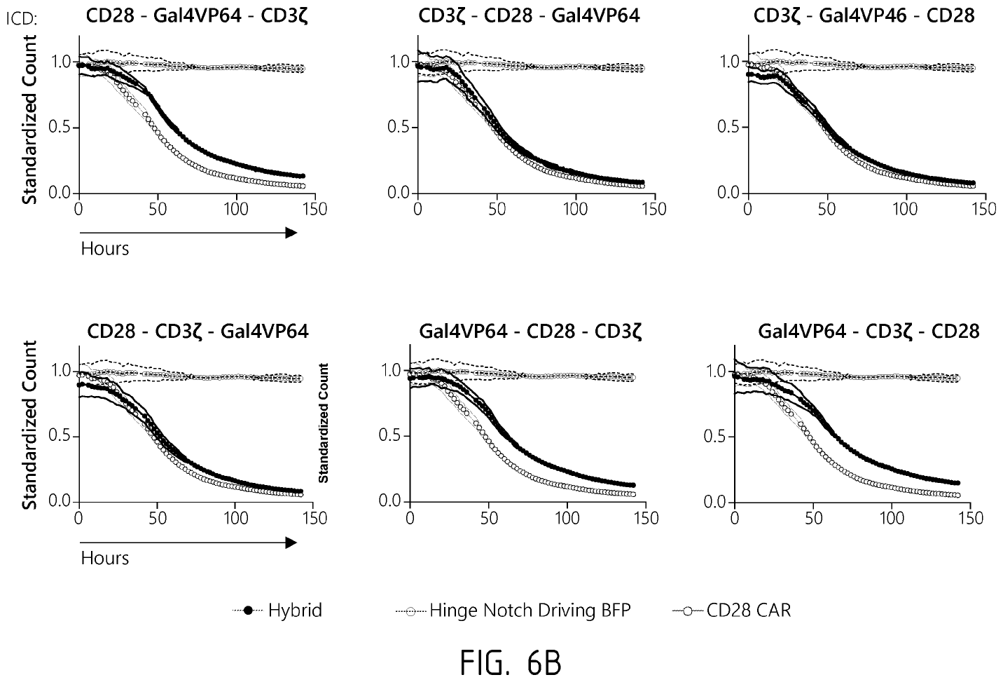

T-cells expressing anti-CD19 receptors with 4-1BB costimulatory domains (FIG. 6A) or CD28 costimulatory domains (FIG. 6B) and the BFP reporter were produced as described above. A549 cells expressing the CD19 ligand and the nuclear stain mkate2 were allowed to adhere to a 96 well flat bottom plate for 24 hours, then transduced T cells were added at a 1:1 ratio. The plate was incubated in an Incucyte, which captured plate images and fluorescence every 2 hours for 5 days. Imaging software was used to calculate the number of A549 CD19+ mkate2+ cells in culture at each time point. For each experimental group, the A549 cell count was normalized to that of the Hinge Notch experimental group. As demonstrated, the Hybrid SynNotch CAR T cells kill target cells at similar rates as the CAR T cells. This indicates that, in this in vitro setting, the Hybrid SynNotch CARs induce T cell activation and cytotoxic programs that are sufficient to cause target cell killing over a period of multiple days.

### Example 8: Hybrid SynNotch Car In Vivo Efficacy

This Example shows the in vivo efficacy of the exemplary hybrid SynNotch CARs provided herein.

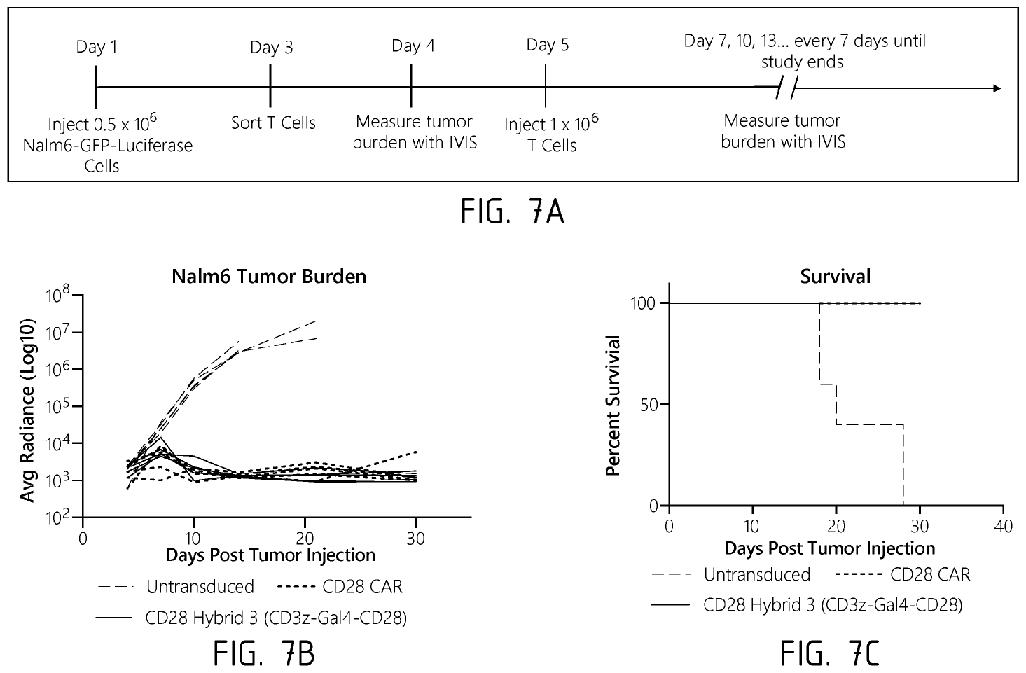

FIG. 7A describes the experimental timeline. NOD.Cg-PrkdcscidIl2rgtm1Wjl/SzJ (NSG) mice were dosed intravenously with 0.5×106 Nalm6-Luc-GFP tumor cells. Bulk CD3+ T-cells were co-transduced with the anti-CD19 hybrid SynNotch CAR with the CD3ζ-Gal4VP64-CD28 intracellular domain and the BFP reporter as described above. In brief, 0.5×106 transduced CD3+ T cells were dosed to animals via retro-orbital injection 4 days post tumor injection. FIG. 7B shows the tumor burden measured via bioluminescence imaging of luciferase secreting tumor cells using an IVIS Spectrum and FIG. 7C shows the survival curve of the experimental mice. This data demonstrates Hybrid SynNotch CAR T cells are effective in clearing Nalm6 tumors in vivo similarly to CAR T cells with a CD28 costimulatory domain. As demonstrated through bioluminescence, the Nalm6 tumor burden initially grows in both Hybrid SynNotch CAR and CAR T cell treated groups, however the tumor burden is reduced, and eventually cleared in both treated groups at approximately day 10 post tumor injection. As demonstrated in the survival curves, animals in the Hybrid SynNotch CAR and CAR groups survive until the end of the study, while the animals dosed with untransduced T cells succumb to their disease between days 18-28 after tumor injection. These data indicate that the Hybrid SynNotch CAR T cells are effective in controlling and clearing tumor burden in vivo.

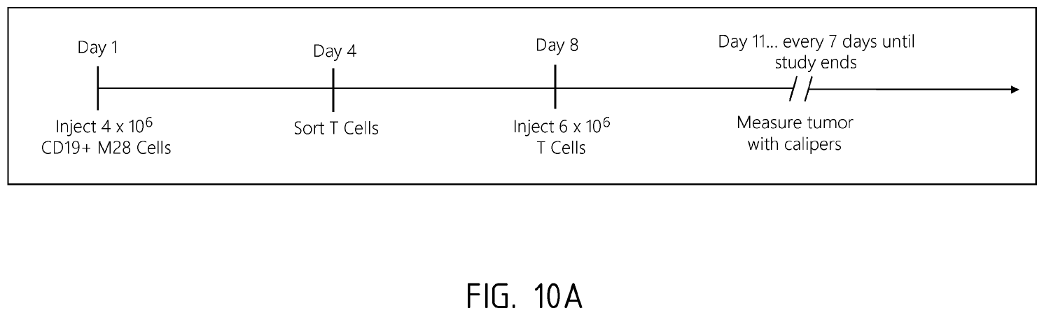

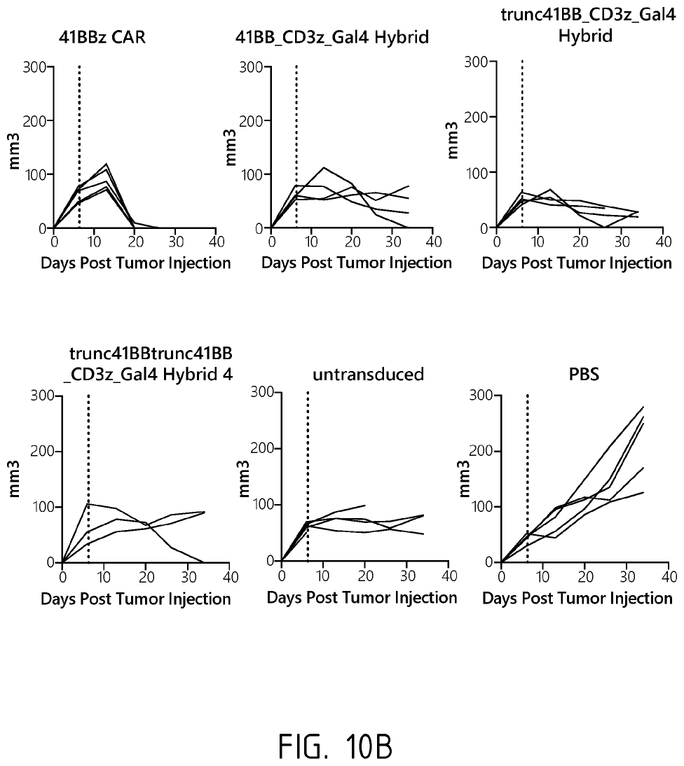

Similarly, FIG. 10A shows a description of experimental timeline. NOD.Cg-PrkdcscidIl2rgtm1Wjl/SzJ (NSG) mice were dosed subcutaneously with 4×106 CD19 ligand expressing M28 tumor cells. Bulk CD3+ T-cells were co-transduced with the anti-CD19 Hybrid SynNotch CAR with ICDs as indicated in 10B and the BFP reporter as described above. In brief, 6×106 transduced CD3+ T cells were dosed to animals via retro-orbital injection 7 days post tumor injection. FIG. 10B shows tumor volume assessed via caliper measurements weekly. These data indicate that the CD19 targeted Hybrid SynNotch CAR T cells, particularly those including the trunc41BB domain, can induce tumor depletion compared to PBS negative control.

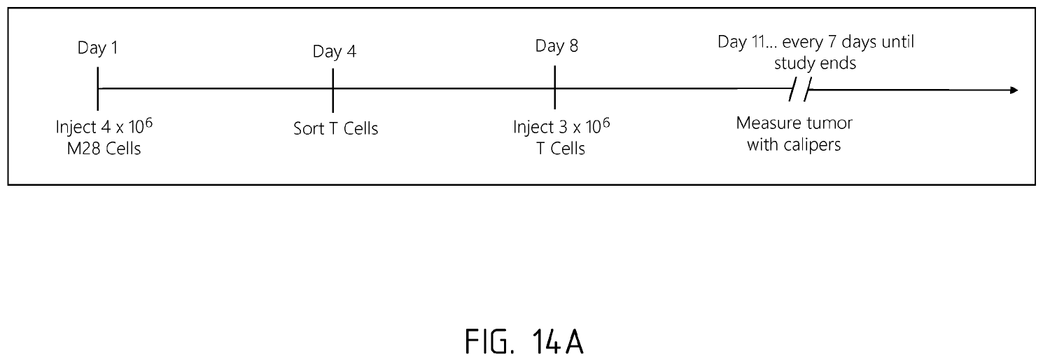

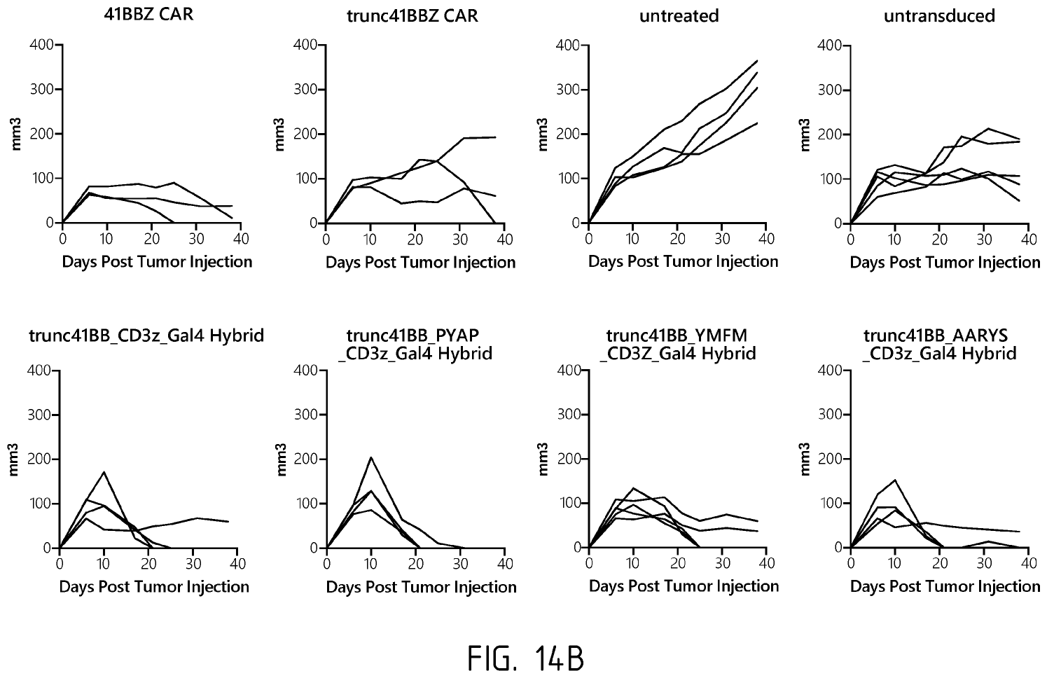

Further, FIG. 14A shows a description of experimental timeline. NOD.Cg-PrkdcscidIl2rgtm1Wjl/SzJ (NSG) mice were dosed subcutaneously with 4×106 CD19 expressing M28 tumor cells. Bulk CD3+ T-cells were co-transduced with the anti-CD19 Hybrid SynNotch CAR with ICDs as indicated in 14B and the BFP reporter as described above. In brief, 3×10⁶ transduced CD3+ T cells were dosed to animals via retro-orbital injection 7 days post tumor injection. FIG. 14B shows tumor volume assessed via caliper measurements weekly. These data indicate that the third generation CD19 targeted Hybrid SynNotch CAR T cells can induce tumor depletion similar to that of a second generation CD19 targeted CAR.

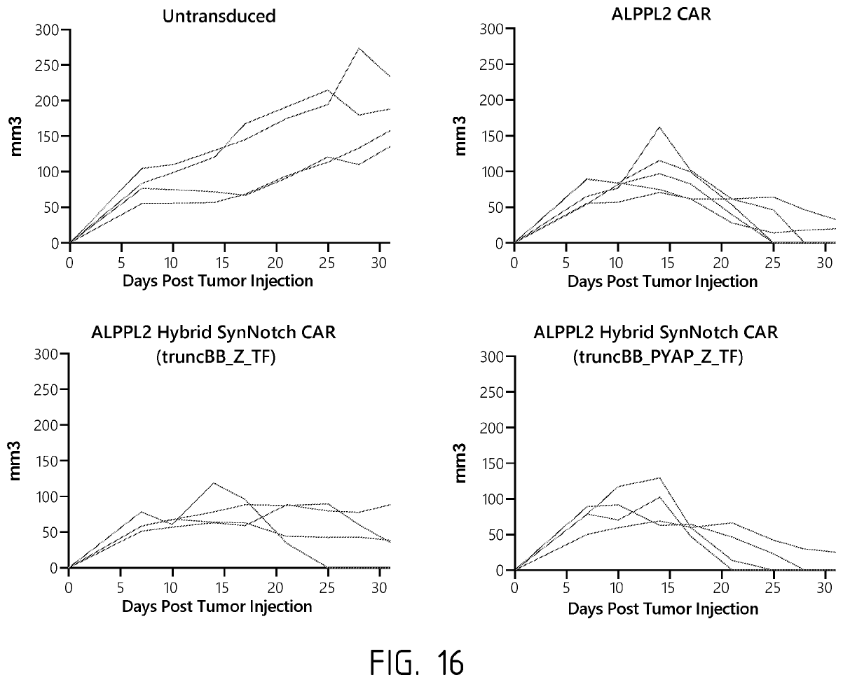

Finally, FIG. 16 shows ALPPL2 Targeted Hybrid SynNotch CAR In Vivo Efficacy. NOD.Cg-PrkdcscidIl2rgtm1Wjl/SzJ (NSG) mice were dosed subcutaneously with 4×106 M28 tumor cells as described in FIG. 14. Bulk CD3+ T-cells were co-transduced with anti-ALPPL2 CAR or the anti-ALPPL2 Hybrid SynNotch CAR with ICDs as indicated in figure and the BFP reporter as described in FIG. 2. 3×106 transduced CD3+ T cells were dosed to animals via retro-orbital injection 7 days post tumor injection. Tumor volume was assessed via caliper measurements weekly. These data indicate that the third generation ALPPL2 targeted Hybrid SynNotch CAR T cells can induce tumor depletion similar to that of a second generation ALPPL2 targeted CAR.

### Example 9: Minimized 4-1BB or CD28 Variants Improve NF-KB Signaling and Reduce Noise

This example shows the improved NF-kB Signaling and reduced noise conferred by a 4-1BB variant and a CD28 variant.

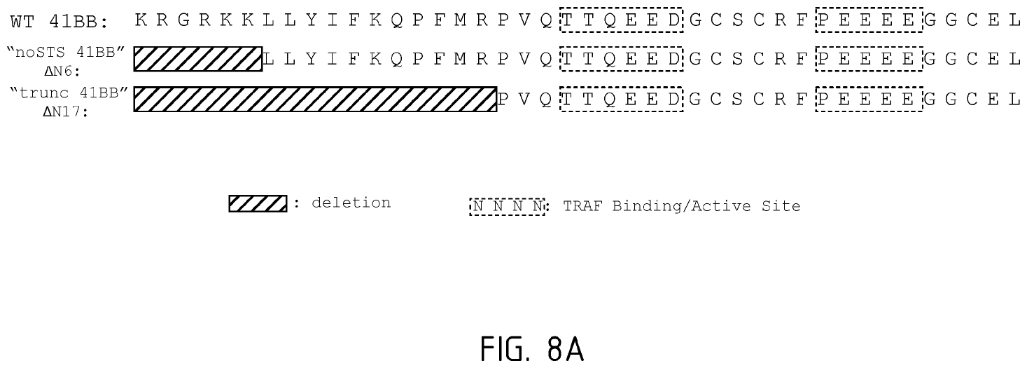

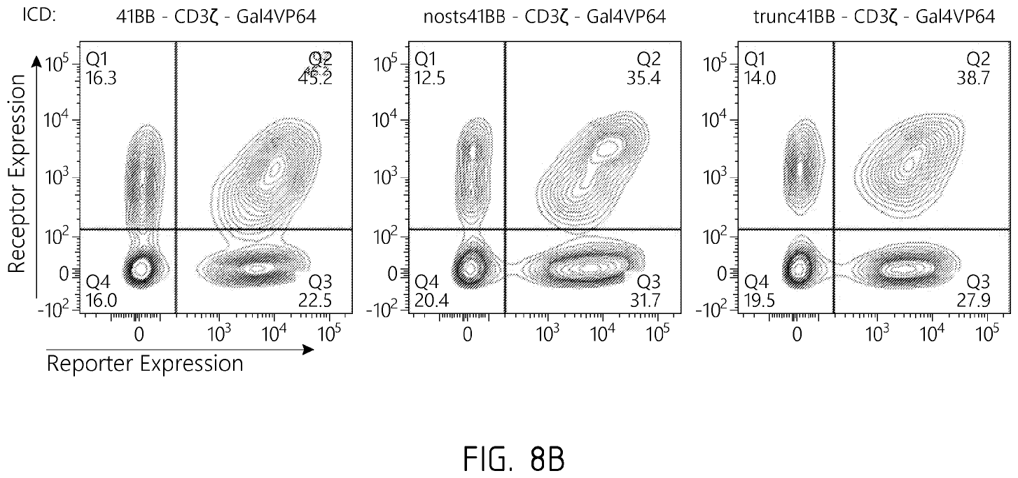

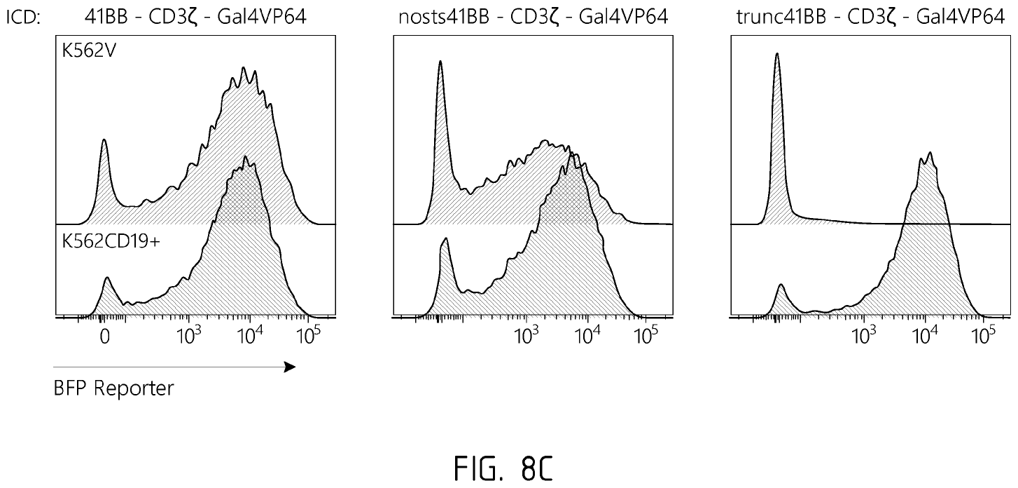

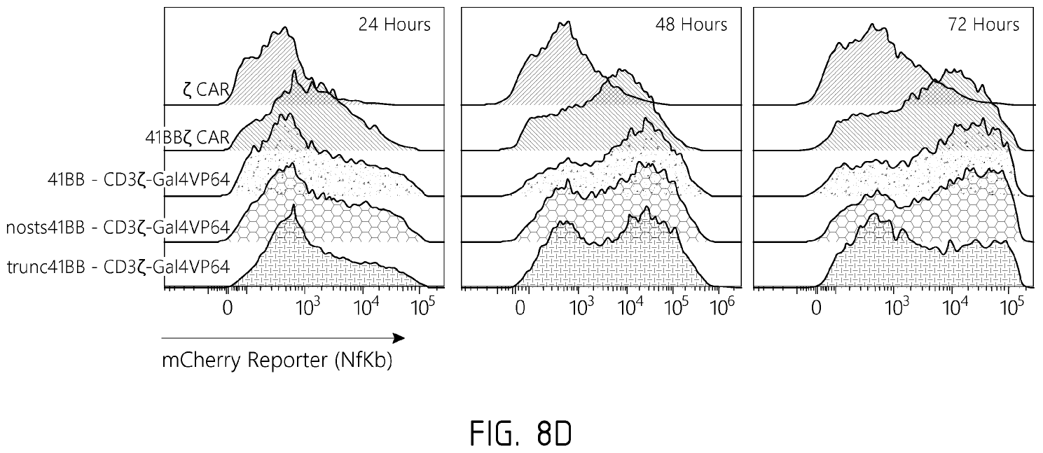

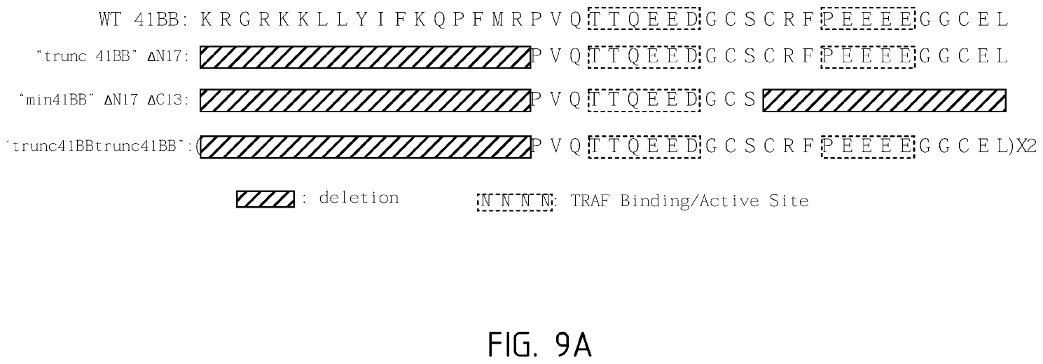

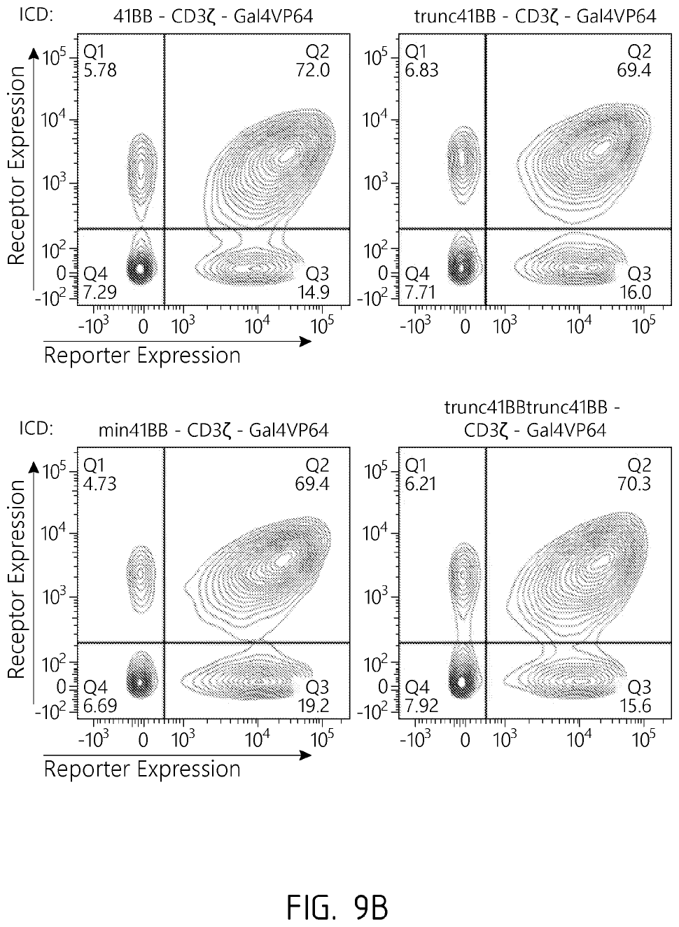

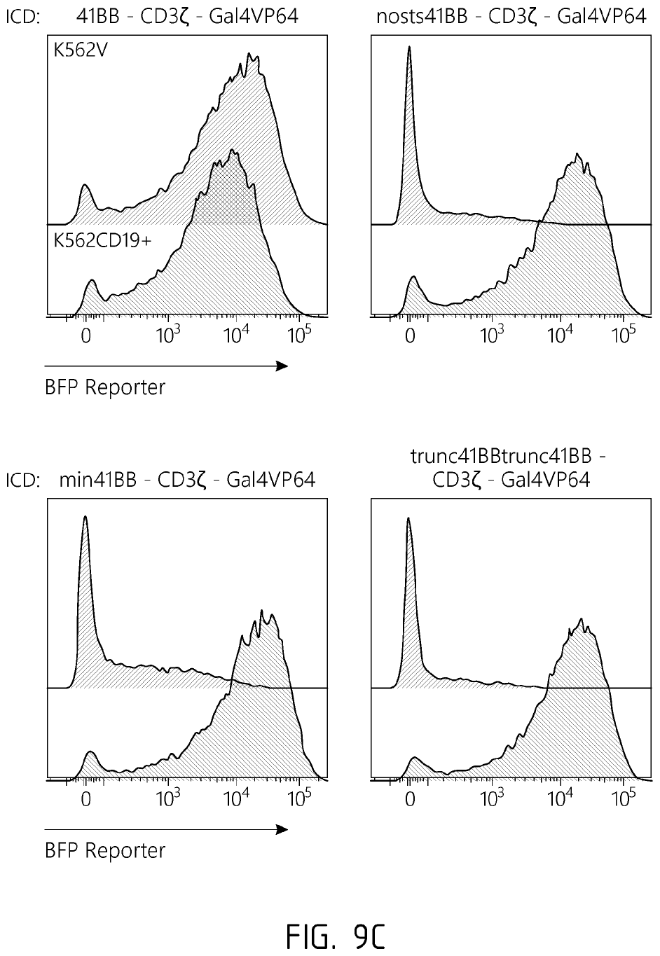

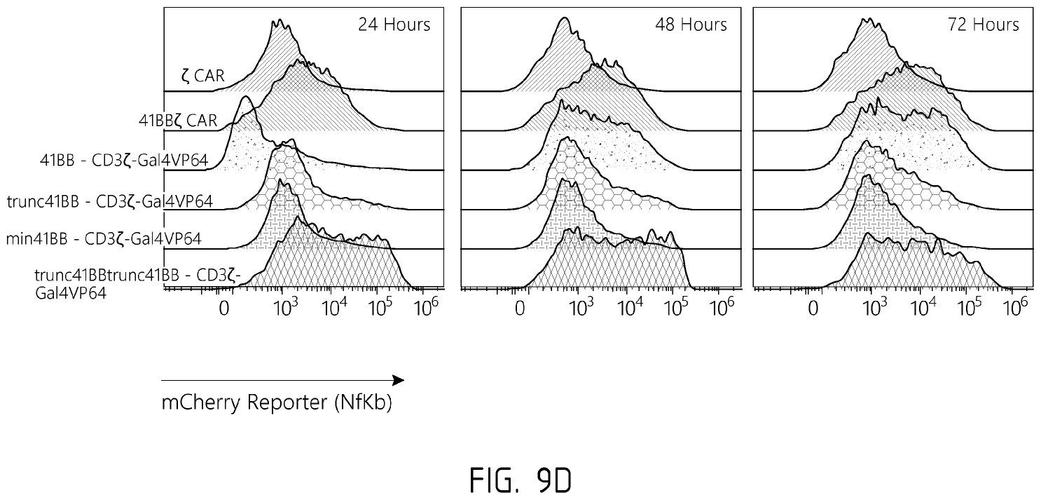

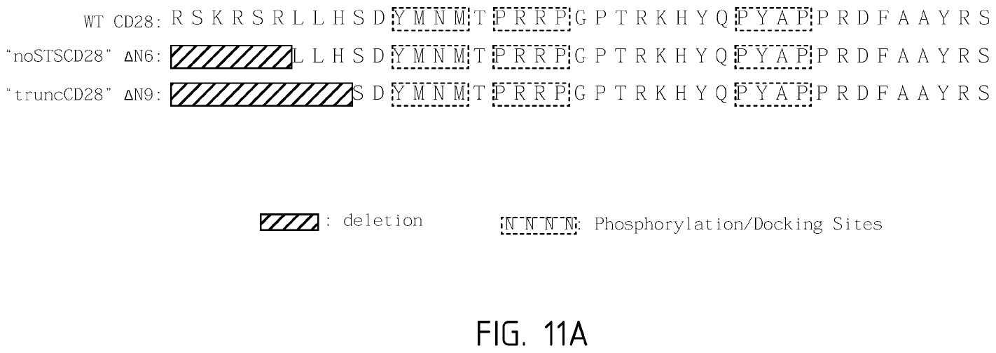

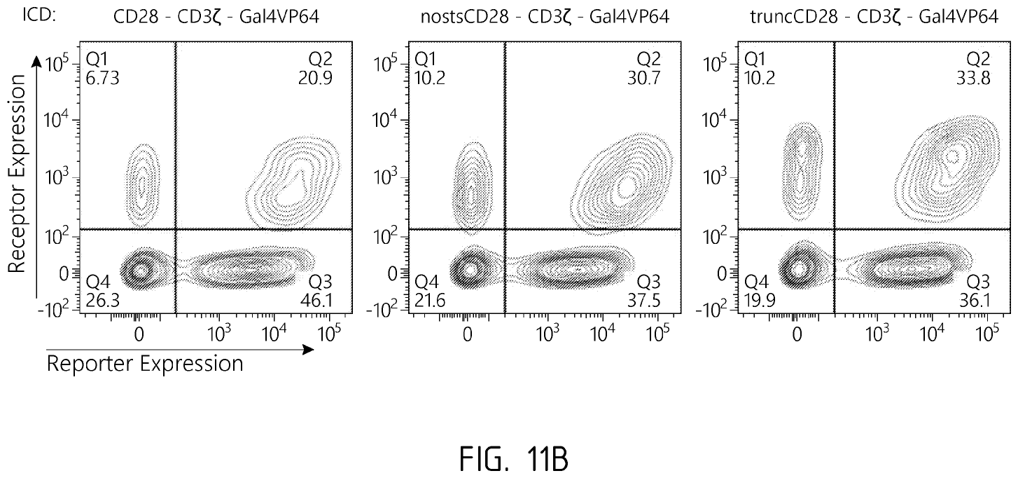

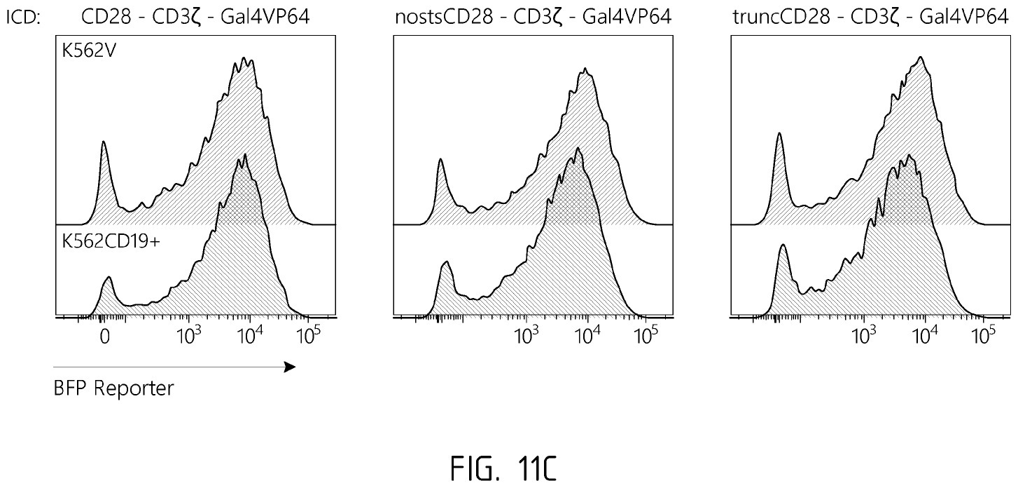

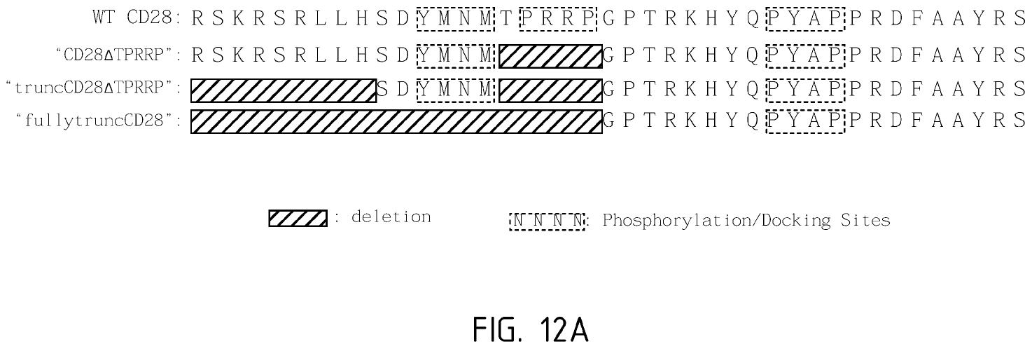

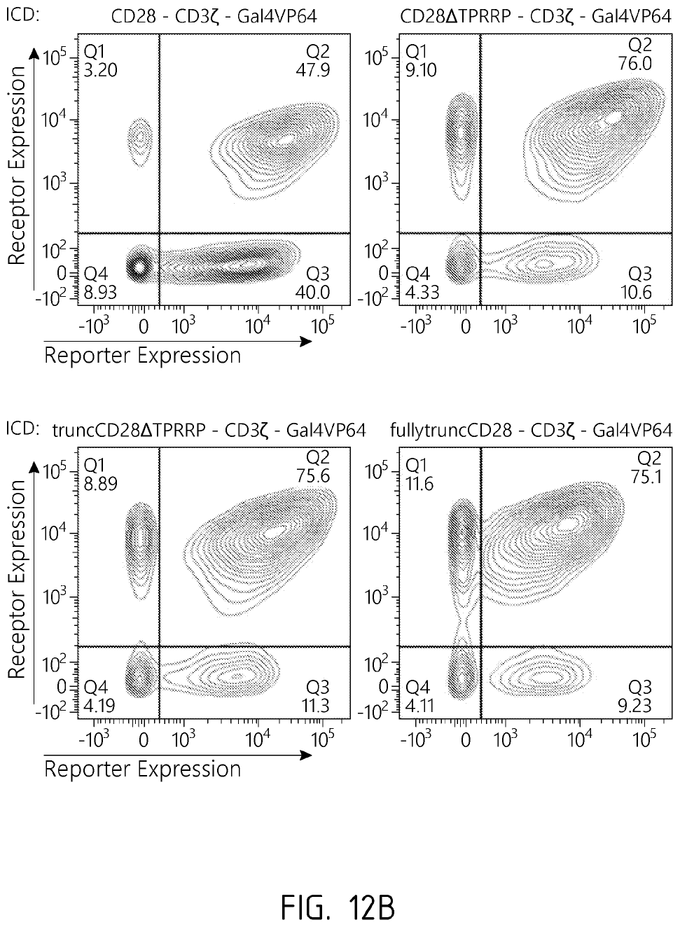

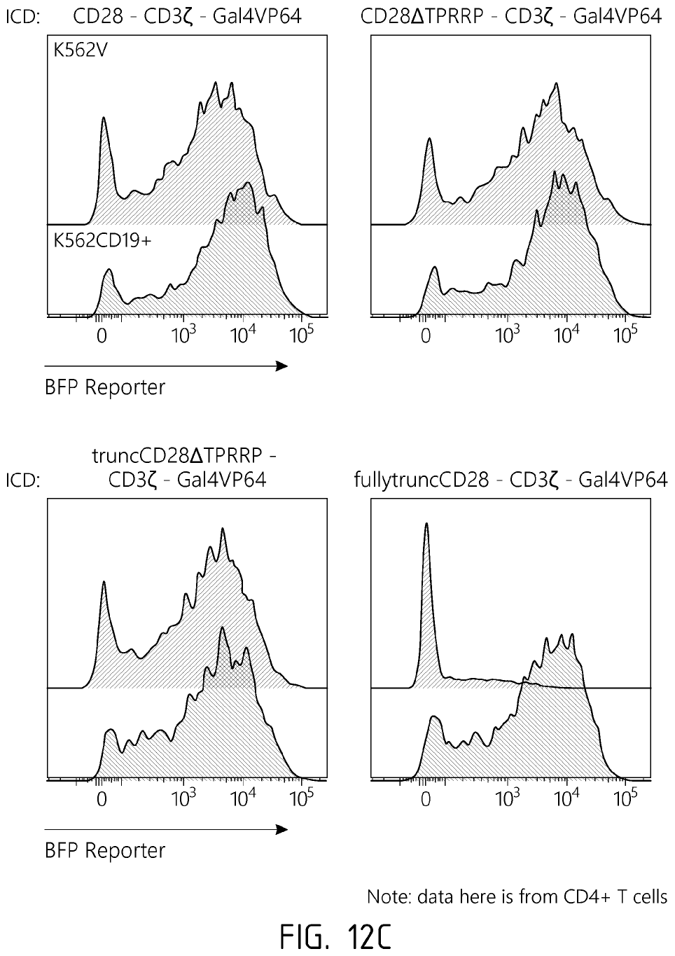

FIG. 8A shows the alignment of wildtype 4-1BB and 4-1BB variants, depicting the amino acids deleted to create the “no STS” and “trunc” 4-1BB costimulatory domains. FIG. 9A shows the alignment of wildtype 4-1BB and 4-1BB variants, depicting the amino acids deleted to create the “trunc 41BB”, “min41BB”, and “trunc41BBtrunc41BB” costimulatory domains. FIGS. 11A and 12A show the alignment of wildtype CD28 and CD28 variants, depicting the amino acids deleted to create the “no STS”, “trunc”, “CD28ΔTPRRP”, “truncCD28ΔTPRRP”, and “fullytruncCD28” CD28 costimulatory domains.

T cells were co-transduced to express the anti-CD19 hybrid SynNotch CAR with either 4-1BB variants or CD28 variants and the BFP reporter as described above. Circuit induction was assessed as described above. FIGS. 8B, 9B, 11B, and 12B demonstrate that the Hybrid SynNotch CARs with 4-1BB variants or CD28 variants are expressed on the surface of the T cell after induction at similar rates as the Hybrid SynNotch CAR with wild type 4-1BB. FIGS. 8C, 9C, 11C, and 12C demonstrate that the Hybrid SynNotch CARs with “noSTS” 4-1BB, “trunc” 4-1BB, “min41BB”, “trunc41bbtrunc41bb”, “no STS” CD28, “trunc” CD28, and “CD28ΔTPRRP”, “truncCD28ΔTPRRP”, and “fullytruncCD28” have less antigen independent induction of the transcriptional circuit, while maintaining the antigen-dependent induction of the inducible transcriptional circuit. This data set indicates that antigen-independent transcriptional regulation of these receptors can be mitigated through modification of the 4-1BB or CD28 costimulatory domains, without diminishing the antigen-dependent activity of the receptor.

A Jurkat cell line was transduced to express an mCherry reporter under a common promoter for NF-κB. This NF-κB reporter cell line was then transduced with anti-CD19 hybrid SynNotch CARs, and co-cultured with K562 cells expressing CD19. mCherry expression was assessed as a proxy for NF-κB activity at 24, 48 and 72 hours post co-culture via flow cytometry. FIGS. 8D and 9D show that the strength of NF-κB signaling induced by the 4-1BB costimulatory domains is unaffected when the 4-1BB domains are modified to remove the STS (“no STS”), the first 17 amino acids of the domain (“trunc”), or the first 17 amino acids of the domain and the the last 13 amino acids of the domain (“min41BB”). These data, in combination with the circuit induction data, indicate that modifications to the 4-1BB costimulatory domain can optimize Hybrid SynNotch CARs with antigen-independent activity, resulting in improved designs that are capable of both antigen dependent transcriptional circuit induction and T cell signaling.

A summary of the expression and T cell activation activities of the hybrid SynNotch CARs described above are provided in Table 8 below.

TABLE 8
Test Result Description.

| Plasmid ID | Receptor | Circuit Expression | T Cell Activation |
|---|---|---|---|
| pRL_2080 | antiCD19scFv-CD8Hinge2-Notch1TMD- | N/A | N/A |
| | Notch2STS-CD28-Gal4VP64-CD3zeta | | |
| pRL_2081 | antiCD19scFv-CD8Hinge2-Notch1TMD- | N/A | N/A |
| | Notch2STS-CD3zeta-CD28-Gal4VP64 | | |
| pRL_2082 | antiCD19scFv-CD8Hinge2-Notch1TMD- | N/A | N/A |
| | Notch2STS-CD3zeta-Gal4VP64-CD28 | | |
| pRL_2083 | antiCD19scFv-CD8Hinge2-Notch1TMD- | N/A | N/A |
| | Notch2STS-CD28-CD3zeta-Gal4VP64 | | |
| pRL_2084 | antiCD 19scFv-CD8Hinge2-Notch1TMD- | N/A | N/A |
| | Notch2STS-Gal4VP64-CD28-CD3zeta | | |
| pRL 2085 | antiCD19scFv-CD8Hinge2-Notch1TMD- | N/A | N/A |
| | Notch2STS-Gal4VP64-CD3zeta-CD28 | | |
| pRL_2103 | antiCD19scFv-CD8Hinge2-Notch1TMD- | Circuit induction is | Quiet at baseline, |
| | Notch2STS-CD28-Gal4VP64-CD3zeta | noisy at baseline. | induces expression |
| | | Receptor induces | of T cell activation |
| | | very high (>90%) | markers similar to |
| | | circuit expression in | CAR in the |
| | | presence of antigen | presence of |
| | | | antigen. |
| pRL_2104 | antiCD19scFv-CD8Hinge2-Notch1TMD- | Circuit induction is | Quiet at baseline, |
| | Notch2STS-CD3zeta-CD28-Gal4VP64 | noisy at baseline. | induces expression |
| | | Receptor induces | of T cell activation |
| | | high (>80%) circuit | markers similar to |
| | | expression in | CAR in the |
| | | presence of antigen | presence of |
| | | | antigen. |
| pRL_2105 | antiCD19scFv-CD8Hinge2-Notch1TMD- | Circuit induction is | Quiet at baseline, |
| | Notch2STS-CD3zeta-Gal4VP64-CD28 | quiet at baseline. | induces expression |
| | | Receptor induces | of T cell activation |
| | | low to medium | markers similar to |
| | | (~40%) circuit | CAR in the |
| | | expression in | presence of |
| | | presence of antigen | antigen. |
| pRL_2106 | antiCD19scFv-CD8Hinge2-Notch1TMD- | Circuit induction is | Quiet at baseline, |
| | Notch2STS-CD28-CD3zeta-Gal4VP64 | noisy at baseline. | induces expression |
| | | Receptor induces | of T cell activation |
| | | high (>80%) circuit | markers similar to |
| | | expression in | CAR in the |
| | | presence of antigen | presence of |
| | | | antigen. |
| pRL_2107 | antiCD19scFv-CD8Hinge2-Notch1TMD- | Circuit induction is | Quiet at baseline, |
| | Notch2STS-Gal4VP64-CD28-CD3zeta | noisy at baseline. | induces expression |
| | | Receptor induces | of T cell activation |
| | | high (>80%) circuit | markers similar to |
| | | expression in | CAR in the |
| | | presence of antigen | presence of |
| | | | antigen. |
| pRL_2108 | antiCD19scFv-CD8Hinge2-Notch1TMD- | Circuit induction is | Quiet at baseline, |
| | Notch2STS-Gal4VP64-CD3zeta-CD28 | noisy at baseline. | induces expression |
| | | Receptor induces | of T cell activation |
| | | high (>80%) circuit | markers similar to |
| | | expression in | CAR in the |
| | | presence of antigen | presence of |
| | | | antigen. |
| pRL_2144 | antiCD19scFv-CD8Hinge2-Notch1TMD- | Circuit induction is | Quiet at baseline, |
| | Notch2STS-noSTSCD28-CD3z-Gal4VP64 | noisy at baseline. | induces expression |
| | | Receptor induces | of T cell activation |
| | | high (>80%) circuit | markers similar to |
| | | expression in | CAR in the |
| | | presence of antigen | presence of |
| | | | antigen. |
| pRL_2146 | antiCD19scFv-CD8Hinge2-Notch1TMD- | Circuit induction is | Quiet at baseline, |
| | Notch2STS-truncCD28-CD3z-Gal4VP64 | noisy at baseline. | induces expression |
| | | Receptor induces | of T cell activation |
| | | high (>80%) circuit | markers similar to |
| | | expression in | CAR in the |
| | | presence of antigen | presence of |
| | | | antigen. |
| pRL_2205 | antiCD19scFv-CD8Hinge2-Notch1TMD- | Circuit induction is | Quiet at baseline, |
| | Notch2STS-CD28delTPRRP-CD3z- | noisy at baseline. | induces expression |
| | Gal4VP64 | Receptor induces | of T cell activation |
| | | high (>80%) circuit | markers similar to |
| | | expression in | CAR in the |
| | | presence of antigen | presence of |
| | | | antigen. |
| pRL_2206 | antiCD19scFv-CD8Hinge2-Notch1TMD- | Circuit induction is | Quiet at baseline, |
| | Notch2STS-truncCD28delTPRRP-CD3z- | quieter at baseline. | induces expression |
| | Gal4VP64 | Receptor induces | of T cell activation |
| | | high (>80%) circuit | markers similar to |
| | | expression in | CAR in the |
| | | presence of antigen | presence of |
| | | | antigen. |
| pRL_2207 | antiCD19scFv-CD8Hinge2-Notch1TMD- | Circuit induction is | Quiet at baseline, |
| | Notch2STS-fullytruncCD28-CD3z- | quieter at baseline. | induces expression |
| | Gal4VP64 | Receptor induces | of T cell activation |
| | | high (>80%) circuit | markers similar to |
| | | expression in | CAR in the |
| | | presence of antigen | presence of |
| | | | antigen. |
| pRL_2073 | antiCD19scFv-CD8Hinge2-Notch1TMD- | Circuit induction is | Quiet at baseline, |
| | Notch2STS-41BB-Gal4VP64-CD3zeta | noisy at baseline. | induces expression |
| | | Receptor induces | of T cell activation |
| | | high (>70%) circuit | markers similar to |
| | | expression in | CAR in the |
| | | presence of antigen | presence of |
| | | | antigen. |
| pRL_2074 | antiCD19scFv-CD8Hinge2-Notch1TMD- | Circuit is quiet at | Quiet at baseline, |
| | Notch2STS-CD3zeta-41BB-Gal4VP64 | baseline. Receptor | induces expression |
| | | induces medium to | of T cell activation |
| | | high >60%) circuit | markers similar to |
| | | expression in | CAR in the |
| | | presence of antigen | presence of |
| | | | antigen. |
| pRL_2075 | antiCD19scFv-CD8Hinge2-Notch1TMD- | Circuit is quiet at | Quiet at baseline, |
| | Notch2STS-CD3zeta-Gal4VP64-41BB | baseline. Receptor | induces expression |
| | | induces low (~35%) | of T cell activation |
| | | circuit expression in | markers similar to |
| | | presence of antigen | CAR in the |
| | | | presence of |
| | | | antigen. |
| pRL_2076 | antiCD19scFv-CD8Hinge2-Notch1TMD- | Circuit induction is | Quiet at baseline, |
| | Notch2STS-41BB-CD3zeta-Gal4VP64 | very noisy at | induces expression |
| | | baseline. Receptor | of T cell activation |
| | | induces high (>80%) | markers similar to |
| | | circuit expression in | CAR in the |
| | | presence of antigen | presence of |
| | | | antigen. |
| pRL_2077 | antiCD19scFv-CD8Hinge2-Notch1TMD- | Circuit induction is | Quiet at baseline, |
| | Notch2STS-Gal4VP64-41BB-CD3zeta | quiet at baseline. | induces expression |
| | | Receptor induces | of T cell activation |
| | | medium to high | markers similar to |
| | | (>60%) circuit | CAR in the |
| | | expression in | presence of |
| | | presence of antigen | antigen. |
| pRL_2078 | antiCD19scFv-CD8Hinge2-Notch1TMD- | Circuit induction is | Quiet at baseline, |
| | Notch2STS-Gal4VP64-CD3zeta-41BB | quiet at baseline. | induces expression |
| | | Receptor induces | of T cell activation |
| | | high (>80%) circuit | markers similar to |
| | | expression in | CAR in the |
| | | presence of antigen | presence of |
| | | | antigen. |
| pRL_2097 | antiCD19scFv-CD8Hinge2-Notch1TMD- | Circuit induction is | Quiet at baseline, |
| | Notch2STS-41BB-Gal4VP64-CD3zeta | noisy at baseline. | induces expression |
| | | Receptor induces | of T cell activation |
| | | very high (>90%) | markers similar to |
| | | circuit expression in | CAR in the |
| | | presence of antigen | presence of |
| | | | antigen. |
| pRL_2098 | antiCD19scFv-CD8Hinge2-Notch1TMD- | Circuit is quiet at | Quiet at baseline, |
| | Notch2STS-CD3zeta-41BB-Gal4VP64 | baseline. Receptor | induces expression |
| | | induces medium to | of T cell activation |
| | | high (>70%) circuit | markers similar to |
| | | expression in | CAR in the |
| | | presence of antigen | presence of |
| | | | antigen. |
| pRL_2099 | antiCD19scFv-CD8Hinge2-Notch1TMD- | Circuit is quiet at | Quiet at baseline, |
| | Notch2STS-CD3zeta-Gal4VP64-41BB | baseline. Receptor | induces expression |
| | | induces low (~35%) | of T cell activation |
| | | circuit expression in | markers similar to |
| | | presence of antigen | CAR in the |
| | | | presence of |
| | | | antigen. |
| pRL_2100 | antiCD19scFv-CD8Hinge2-Notch1TMD- | Circuit induction is | Quiet at baseline, |
| | Notch2STS-41BB-CD3zeta-Gal4VP64 | very noisy at | induces expression |
| | | baseline. Receptor | of T cell activation |
| | | induces very high | markers similar to |
| | | (>90%) circuit | CAR in the |
| | | expression in | presence of |
| | | presence of antigen | antigen. |
| pRL_2101 | antiCD19scFv-CD8Hinge2-Notch1TMD- | Circuit induction is | Quiet at baseline, |
| | Notch2STS-Gal4VP64-41BB-CD3zeta | quiet at baseline. | induces expression |
| | | Receptor induces | of T cell activation |
| | | high (>80%) circuit | markers similar to |
| | | expression in | CAR in the |
| | | presence of antigen | presence of |
| | | | antigen. |
| pRL_2102 | antiCD19scFv-CD8Hinge2-Notch1TMD- | Circuit induction is | Quiet at baseline, |
| | Notch2STS-Gal4VP64-CD3zeta-41BB | quiet at baseline. | induces expression |
| | | Receptor induces | of T cell activation |
| | | very high (>90%) | markers similar to |
| | | circuit expression in | CAR in the |
| | | presence of antigen | presence of |
| | | | antigen. |
| pRL_2140 | antiCD19scFv-CD8Hinge2-Notch1TMD- | Circuit induction is | Quiet at baseline, |
| | Notch2STS-noSTS41BB-CD3zeta- | noisy at baseline, | induces expression |
| | Gal4VP64 | but less so than | of T cell activation |
| | | pRL_2100. Receptor | markers similar to |
| | | induces high (>85%) | CAR in the |
| | | circuit expression in | presence of |
| | | presence of antigen. | antigen. |
| | | Somewhat improved | |
| | | version of | |
| | | pRL_2100, where | |
| | | noise is high at | |
| | | baseline. | |
| pRL_2142 | antiCD19scFv-CD8Hinge2-Notch1TMD- | Circuit induction is | Quiet at baseline, |
| | Notch2STS-trunc41BB-CD3zeta- | quiet at baseline. | induces expression |
| | Gal4VP64 | Receptor induces | of T cell activation |
| | | very high (>90%) | markers similar to |
| | | circuit expression in | CAR in the |
| | | presence of antigen. | presence of |
| | | Much improved | antigen. |
| | | version of | |
| | | pRL_2100, where | |
| | | noise is high at | |
| | | baseline. | |
| pRL_2184 | antiCD19scFv-CD8Hinge2-Notch1TMD- | Circuit induction is | Quiet at baseline, |
| | Notch2STS-trunc41BBtrunc41BB-CD3z- | quiet at baseline. | induces expression |
| | Gal4VP64 | Receptor induces | of T cell activation |
| | | very high (>90%) | markers similar to |
| | | circuit expression in | CAR in the |
| | | presence of antigen. | presence of |
| | | | antigen. |
| pRL_2185 | antiCD19scFv-CD8Hinge2-Notch1TMD- | | |
| | Notch2STS- | | |
| | trunc41BBtrunc41BBtrunc41BB-CD3z- | | |
| | Gal4VP64 | | |
| pRL_2186 | antiCD19scFv-CD8Hinge2-Notch1TMD- | Circuit induction is | Quiet at baseline, |
| | Notch2STS-min41BB-CD3z-Gal4VP64 | noiser at baseline. | induces expression |
| | | Receptor induces | of T cell activation |
| | | very high (>90%) | markers similar to |
| | | circuit expression in | CAR in the |
| | | presence of antigen. | presence of |
| | | | antigen. |

### Example 10: “Third Generation” Variants

This example shows the reduced noise conferred by a variants, which include one of the CD28 signaling motifs appended to the C terminus of a trunc41BB costimulatory domain.

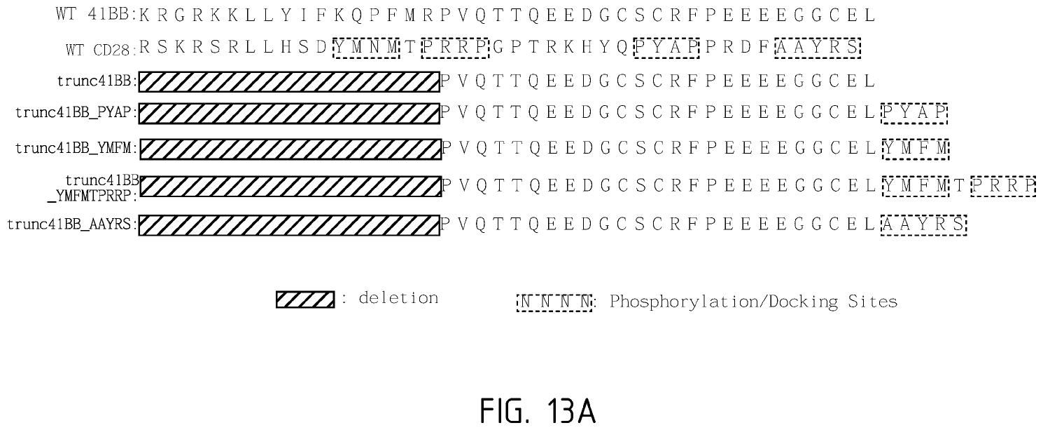

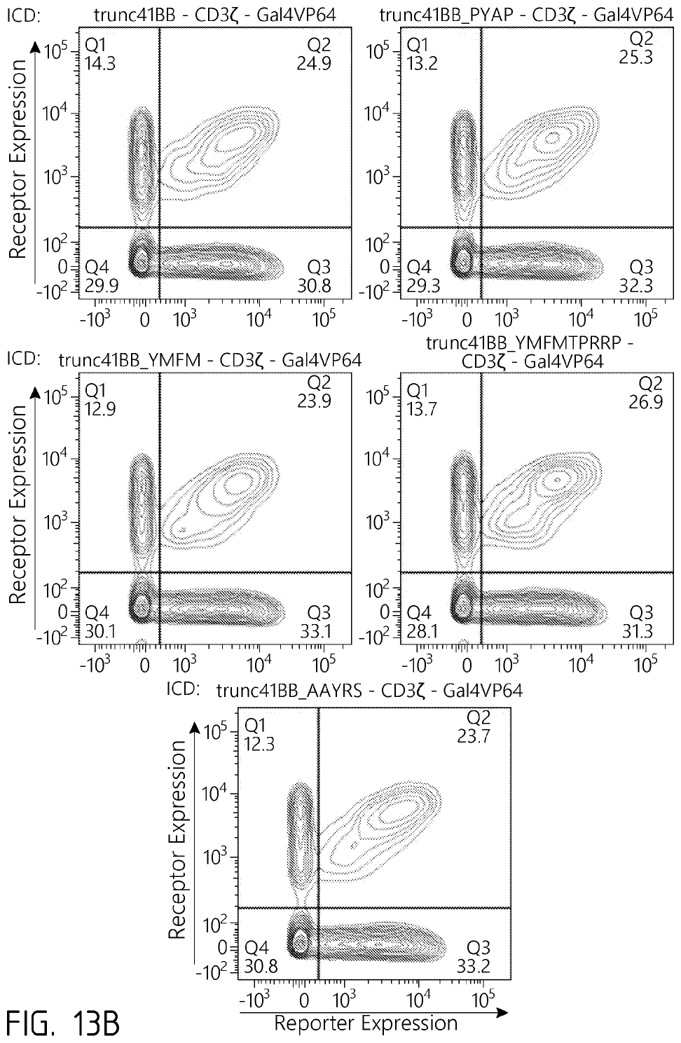

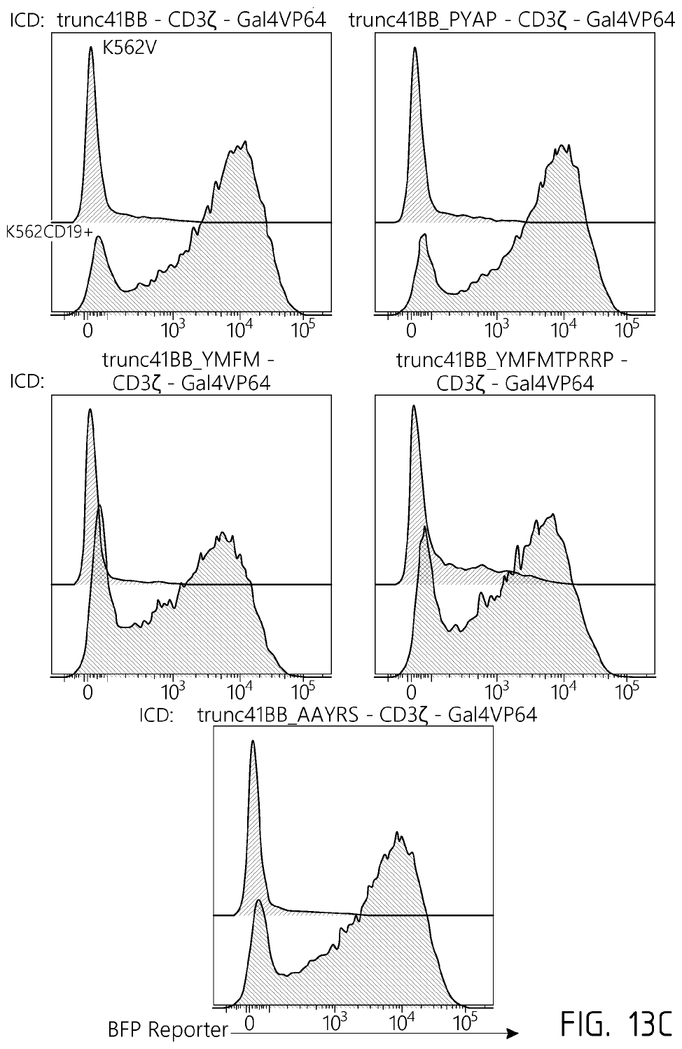

FIG. 13A shows the alignment of wildtype 4-1BB and CD28 and variants, depicting the amino acids deleted and/or added to create the “trunc41BB PYAP”, “trunc41BB_YMFM”, “trunc41BB YMFMTPRRP”, and “trunc41BB AAYRS” costimulatory domains.

T cells were co-transduced to express the anti-CD19 hybrid SynNotch CAR with “third generation” variants and the BFP reporter as described above. Circuit induction was assessed as described above. FIG. 13B demonstrates that the Hybrid SynNotch CARs with one of the CD28 signaling motifs appended to the C terminus of a trunc41BB costimulatory domain are expressed on the surface of the T cell after induction at similar levels as the Hybrid SynNotch CAR with wild type 4-1BB. FIG. 13C demonstrates that the Hybrid SynNotch CARs with “trunc41BB PYAP”, “trunc41BB_YMFM”, “trunc41BB YMFMTPRRP”, and “trunc41BB AAYRS” have low antigen independent induction of the transcriptional circuit and maintain antigen-dependent induction of the inducible transcriptional circuit. These data indicate that antigen independent and antigen-dependent transcriptional regulation of these receptors is unaffected by the addition of signaling motifs from CD28 costimulatory domain.

A summary of the expression and T cell activation activities of the hybrid SynNotch CARs described above are provided in Table 9 below.

TABLE 9
Test Result Description.

| Plasmid ID | Receptor | Circuit Expression | T Cell Activation |
|---|---|---|---|
| pRL_2194 | antiCD19scFv-CD8Hinge2-Notch1TMD- | Circuit induction is | Quiet at baseline, |
| | Notch2STS-trunc41BB-PYAP-CD3z- | quiet at baseline. | induces expression |
| | Gal4VP64 | Receptor induces | of T cell activation |
| | | high circuit | markers similar to |
| | | expression in | CAR in the |
| | | presence of antigen. | presence of |
| | | | antigen. |
| pRL_2195 | antiCD19scFv-CD8Hinge2-Notch1TMD- | Circuit induction is | Quiet at baseline, |
| | Notch2STS-trunc41BB-YMFM-CD3z- | quiet at baseline. | induces expression |
| | Gal4VP64 | Receptor induces | of T cell activation |
| | | high circuit | markers similar to |
| | | expression in | CAR in the |
| | | presence of antigen. | presence of |
| | | | antigen. |
| pRL_2196 | antiCD19scFv-CD8Hinge2-Notch1TMD- | Circuit induction is | Quiet at baseline, |
| | Notch2STS-trunc41BB-YMFMTPRRP- | quiet at baseline. | induces expression |
| | CD3z-Gal4VP64 | Receptor induces | of T cell activation |
| | | high circuit | markers similar to |
| | | expression in | CAR in the |
| | | presence of antigen. | presence of |
| | | | antigen. |
| pRL_2197 | antiCD19scFv-CD8Hinge2-Notch1TMD- | Circuit induction is | Quiet at baseline, |
| | Notch2STS-trunc41BB-AAYRS-CD3z- | quiet at baseline. | induces expression |
| | Gal4VP64 | Receptor induces | of T cell activation |
| | | high circuit | markers similar to |
| | | expression in | CAR in the |
| | | presence of antigen. | presence of |
| | | | antigen. |

### Example 10: BCMA and ALPPL2 Targeted Variants

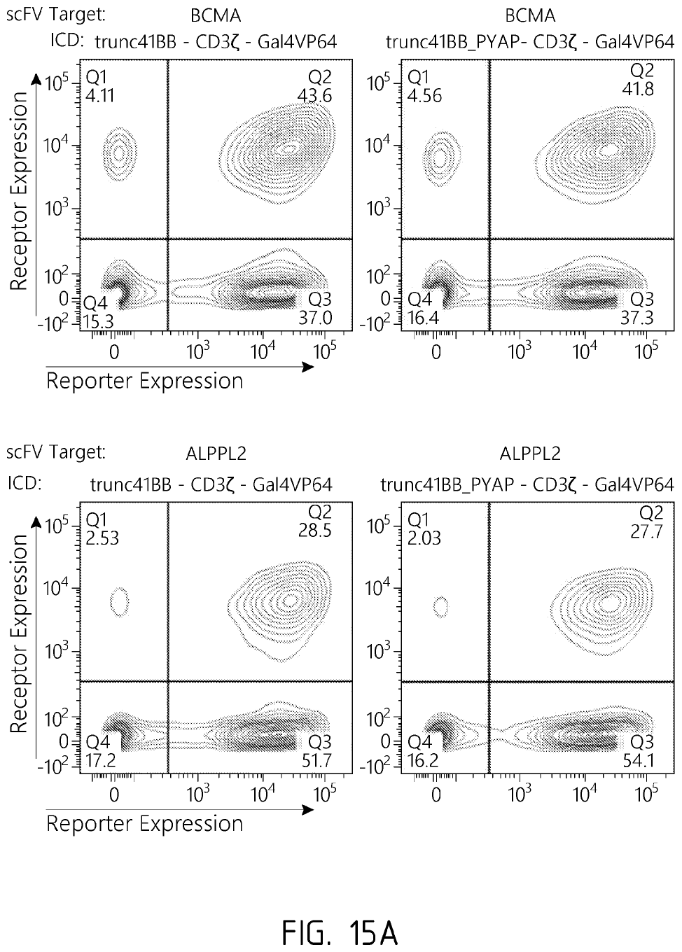

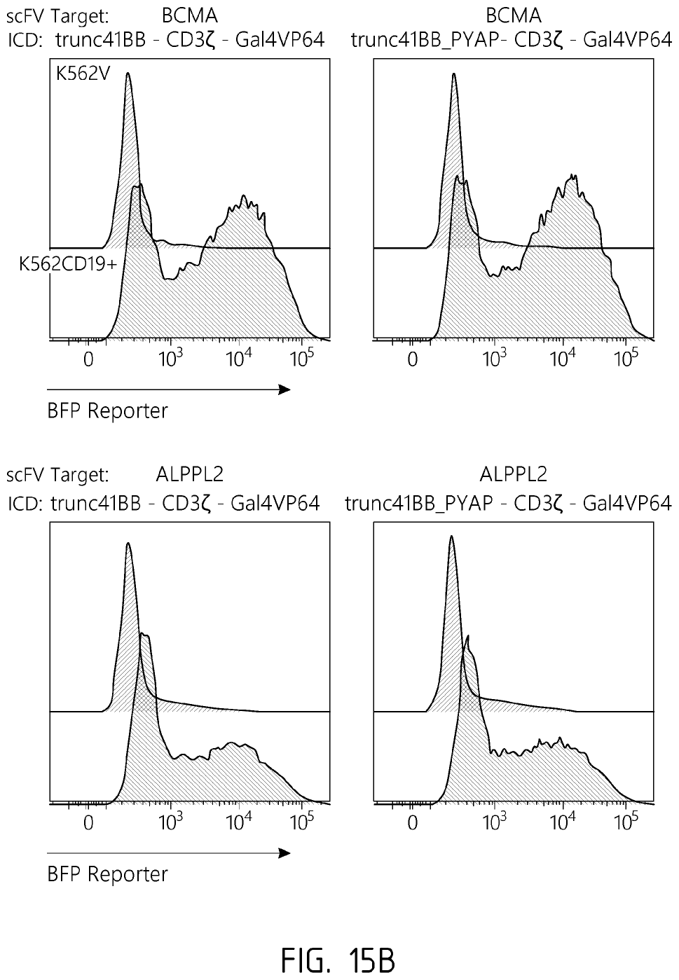

T cells were co-transduced to express the anti-BCMA or anti-ALPPL2 hybrid SynNotch CAR with 41BB costimulatory domain variants and the BFP reporter as described above. Circuit induction was assessed as described above. FIG. 15A demonstrates that the anti-BCMA and anti-ALPPLS Hybrid SynNotch CARs are expressed on the surface of the T cell after induction. FIG. 15B demonstrates that the anti-BCMA and anti-ALPPLS Hybrid SynNotch CARs with “trunc41BB” “trunc41BB PYAP” have less antigen independent induction of the transcriptional circuit, while maintaining the antigen-dependent induction of the inducible transcriptional circuit. This data set indicates that Hybrid SynNotch CAR scaffolds can be fused to other antigen targeting scFvs and maintain antigen-dependent transcriptional regulation of the circuit and T cell activation.

A summary of the expression and T cell activation activities of the hybrid SynNotch CARs described above are provided in Table 10 below.

TABLE 10
Test Result Description.

| Plasmid ID | Receptor | Circuit Expression | T Cell Activation |
|---|---|---|---|
| pRL_2232 | antiBCMAscFv-CD8Hinge2-Notch1TMD- | Circuit induction is | Quiet at baseline, |
| | Notch2STS-trunc41BB-CD3z-Gal4VP64 | quiet at baseline. | induces expression |
| | | Receptor induces | of T cell activation |
| | | very high (>70%) | markers similar to |
| | | circuit expression in | CAR in the |
| | | presence of antigen. | presence of |
| | | | antigen. |
| pRL_2234 | antiALPPL2scFv-CD8Hinge2- | Circuit induction is | Quiet at baseline, |
| | Notch1TMD-Notch2STS-trunc41BB- | quiet at baseline. | induces expression |
| | CD3z-Gal4VP64 | Receptor induces | of T cell activation |
| | | very high (>55%) | markers similar to |
| | | circuit expression in | CAR in the |
| | | presence of antigen. | presence of |
| | | | antigen. |
| pRL_2233 | antiBCMAscFv-CD8Hinge2-Notch1TMD- | Circuit induction is | Quiet at baseline, |
| | Notch2STS-trunc41BB-PYAP-CD3z- | quiet at baseline. | induces expression |
| | Gal4VP64 | Receptor induces | of T cell activation |
| | | high circuit | markers similar to |
| | | expression in | CAR in the |
| | | presence of antigen. | presence of |
| | | | antigen. |
| pRL_2235 | antiALPPL2scFv-CD8Hinge2- | Circuit induction is | Quiet at baseline, |
| | Notch1TMD-Notch2STS-trunc41BB- | quiet at baseline. | induces expression |
| | PYAP-CD3z-Gal4VP64 | Receptor induces | of T cell activation |
| | | high circuit | markers similar to |
| | | expression in | CAR in the |
| | | presence of antigen. | presence of |
| | | | antigen. |

SEQUENCE TABLE

| | | SEQ |
|---|---|---|
| DESCRIPTION | SEQUENCE | ID NO |
| ECD | MALPVTALLLPLALLLHAARPEQKLISEEDLDIQMTQTTSSLSASLGDR | 1 |
| | VTISCRASQDISKYLNWYQQKPDGTVKLLIYHTSRLHSGVPSRFSGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVELKMNSLQTDDTAI | |
| | YYCAKHYYYGGSYAMDYWGQGTSVTVSS | |
| ECD | MALPVTALLLPLALLLHAARPDYKDDDDKDIQMTQTTSSLSASLGDRVT | 2 |
| | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSS | |
| LINKING | TTTPAPRPPTPAPTIASQPLSLRPEAC | 3 |
| POLYPEPTIDE | | |
| TMD | FMYVAAAAFVLLFFVGCGVLL | 4 |
| STS | SKRKRKH | 5 |
| GS Linker | GSGSGSGS | 6 |
| ICD | KRGRKKLLYIFKQPFMRPVQTTQEEDGCSCRFPEEEEGGCEL | 7 |
| ICD | RVKFSRSADAPAYKQGQNQLYNELNLGRREEYDVLDKRRGRDPEMGGKP | 8 |
| | RRKNPQEGLYNELQKDKMAEAYSEIGMKGERRRGKGHDGLYQGLSTATK | |
| | DTYDALHMQALPPR | |
| ICD | MKLLSSIEQACDICRLKKLKCSKEKPKCAKCLKNNWECRYSPKTKRSPL | 9 |
| | TRAHLTEVESRLERLEQLFLLIFPREDLDMILKMDSLQDIKALLTGLFV | |
| | QDNVNKDAVTDRLASVETDMPLTLRQHRISATSSSEESSNKGQRQLTVS | |
| | AAAGGSGGSGGSDALDDFDLDMLGSDALDDEDLDMLGSDALDDEDLDML | |
| | GSDALDDEDLDMLGS | |
| ICD | LLYIFKQPFMRPVQTTQEEDGCSCRFPEEEEGGCEL | 10 |
| ICD | PVQTTQEEDGCSCRFPEEEEGGCEL | 11 |
| ICD | PVQTTQEEDGCSCRFPEEEEGGCELPVQTTQEEDGCSCRFPEEEEGGCE | 12 |
| | L | |
| ICD | PVQTTQEEDGCCRFPEEEEGGCELPVQTTQEEDGCSCRFPEEEEGGCE | 13 |
| | LPVQTTQEEDGCCRFPEEEEGGCEL | |
| ICD | PVQTTQEEDGCS | 14 |
| ICD | RSKRSRLLHSDYMNMTPRRPGPTRKHYQPYAPPRDFAAYRS | 33 |
| FULL | MALPVTALLLPLALLLHAARPEQKLISEEDLDIQMTQTTSSLSASLGDR | 15 |
| SEQUENCE | VTISCRASQDISKYLNWYQQKPDGTVKLLIYHTSRLHSGVPSRFSGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVFLKMNSLQTDDTAI | |
| | YYCAKHYYYGGSYAMDYWGQGTSVTVSSTTTPAPRPPTPAPTIASQPLS | |
| | LRPEACEMYVAAAAFVLLFFVGCGVLLSKRKRKHGSGSGSGSKRGRKKL | |
| | LYIFKQPFMRPVQTTQEEDGCSCRFPEEEEGGCELGSGSGSGSMKLLSS | |
| | IEQACDICRLKKLKCSKEKPKCAKCLKNNWECRYSPKTKRSPLTRAHLT | |
| | EVESRLERLEQLFLLIFPREDLDMILKMDSLQDIKALLTGLEVQDNVNK | |
| | DAVTDRLASVETDMPLTLRQHRISATSSSEESSNKGQRQLTVSAAAGGS | |
| | GGSGGSDALDDFDLDMLGSDALDDFDLDMLGSDALDDEDLDMLGSDALD | |
| | DFDLDMLGSGSGSGSGSRVKFSRSADAPAYKQGQNQLYNELNLGRREEY | |
| | DVLDKRRGRDPEMGGKPRRKNPQEGLYNELQKDKMAEAYSEIGMKGERR | |
| | RGKGHDGLYQGLSTATKDTYDALHMQALPPR | |
| FULL | MALPVTALLLPLALLLHAARPEQKLISEEDLDIQMTQTTSSLSASLGDR | 16 |
| SEQUENCE | VTISCRASQDISKYLNWYQQKPDGTVKLLIYHTSRLHSGVPSRFSGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVFLKMNSLQTDDTAI | |
| | YYCAKHYYYGGSYAMDYWGQGTSVTVSSTTTPAPRPPTPAPTIASQPLS | |
| | LRPEACEMYVAAAAFVLLFFVGCGVLLSKRKRKHGSGSGSGSRVKESRS | |
| | ADAPAYKQGQNQLYNELNLGRREEYDVLDKRRGRDPEMGGKPRRKNPQE | |
| | GLYNELQKDKMAEAYSEIGMKGERRRGKGHDGLYQGLSTATKDTYDALH | |
| | MQALPPRGSGSGSGSKRGRKKLLYIFKQPFMRPVQTTQEEDGCSCREPE | |
| | EEEGGCELGSGSGSGSMKLLSSIEQACDICRLKKLKCSKEKPKCAKCLK | |
| | NNWECRYSPKTKRSPLTRAHLTEVESRLERLEQLELLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDEDL | |
| | DMLGSDALDDEDLDMLGSDALDDEDLDMLGS | |
| FULL | MALPVTALLLPLALLLHAARPEQKLISEEDLDIQMTQTTSSLSASLGDR | 17 |
| SEQUENCE | VTISCRASQDISKYLNWYQQKPDGTVKLLIYHTSRLHSGVPSRFSGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVFLKMNSLQTDDTAI | |
| | YYCAKHYYYGGSYAMDYWGQGTSVTVSSTTTPAPRPPTPAPTIASQPLS | |
| | LRPEACEMYVAAAAFVLLFFVGCGVLLSKRKRKHGSGSGSGSRVKESRS | |
| | ADAPAYKQGQNQLYNELNLGRREEYDVLDKRRGRDPEMGGKPRRKNPQE | |
| | GLYNELQKDKMAEAYSEIGMKGERRRGKGHDGLYQGLSTATKDTYDALH | |
| | MQALPPRGSGSGSGSMKLLSSIEQACDICRLKKLKCSKEKPKCAKCLKN | |
| | NWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSS | |
| | SEESSNKGQRQLTVSAAAGGSGGSGGSDALDDEDLDMLGSDALDDEDLD | |
| | MLGSDALDDFDLDMLGSDALDDEDLDMLGSGSGSGSGSKRGRKKLLYIF | |
| | KQPFMRPVQTTQEEDGCSCRFPEEEEGGCEL | |
| FULL | MALPVTALLLPLALLLHAARPEQKLISEEDLDIQMTQTTSSLSASLGDR | 18 |
| SEQUENCE | VTISCRASQDISKYLNWYQQKPDGTVKLLIYHTSRLHSGVPSRFSGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVELKMNSLQTDDTAI | |
| | YYCAKHYYYGGSYAMDYWGQGTSVTVSSTTTPAPRPPTPAPTIASQPLS | |
| | LRPEACEMYVAAAAFVLLFFVGCGVLLSKRKRKHGSGSGSGSKRGRKKL | |
| | LYIFKQPFMRPVQTTQEEDGCSCRFPEEEEGGCELGSGSGSGSRVKFSR | |
| | SADAPAYKQGQNQLYNELNLGRREEYDVLDKRRGRDPEMGGKPRRKNPQ | |
| | EGLYNELQKDKMAEAYSEIGMKGERRRGKGHDGLYQGLSTATKDTYDAL | |
| | HMQALPPRGSGSGSGSMKLLSSIEQACDICRLKKLKCSKEKPKCAKCLK | |
| | NNWECRYSPKTKRSPLTRAHLTEVESRLERLEQLFLLIFPREDLDMILK | |
| | MDSLQDIKALLTGLFVQDNVNKDAVTDRLASVETDMPLTLRQHRISATS | |
| | SSEESSNKGQRQLTVSAAAGGSGGSGGSDALDDFDLDMLGSDALDDEDL | |
| | DMLGSDALDDFDLDMLGSDALDDEDLDMLGS | |
| FULL | MALPVTALLLPLALLLHAARPEQKLISEEDLDIQMTQTTSSLSASLGDR | 19 |
| SEQUENCE | VTISCRASQDISKYLNWYQQKPDGTVKLLIYHTSRLHSGVPSRFSGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVELKMNSLQTDDTAI | |
| | YYCAKHYYYGGSYAMDYWGQGTSVTVSSTTTPAPRPPTPAPTIASQPLS | |
| | LRPEACEMYVAAAAFVLLFFVGCGVLLSKRKRKHGSGSGSGSMKLLSSI | |
| | EQACDICRLKKLKCSKEKPKCAKCLKNNWECRYSPKTKRSPLTRAHLTE | |
| | VESRLERLEQLELLIFPREDLDMILKMDSLQDIKALLTGLFVQDNVNKD | |
| | AVTDRLASVETDMPLTLRQHRISATSSSEESSNKGQRQLTVSAAAGGSG | |
| | GSGGSDALDDFDLDMLGSDALDDEDLDMLGSDALDDEDLDMLGSDALDD | |
| | FDLDMLGSGSGSGSGSKRGRKKLLYIFKQPFMRPVQTTQEEDGCSCREP | |
| | EEEEGGCELGSGSGSGSRVKFSRSADAPAYKQGQNQLYNELNLGRREEY | |
| | DVLDKRRGRDPEMGGKPRRKNPQEGLYNELQKDKMAEAYSEIGMKGERR | |
| | RGKGHDGLYQGLSTATKDTYDALHMQALPPR | |
| FULL | MALPVTALLLPLALLLHAARPEQKLISEEDLDIQMTQTTSSLSASLGDR | 20 |
| SEQUENCE | VTISCRASQDISKYLNWYQQKPDGTVKLLIYHTSRLHSGVPSRFSGSGS | |
| | GTDYSLTISNLEQEDIATYFCQQGNTLPYTFGGGTKLEITGGGGGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVELKMNSLQTDDTAI | |
| | YYCAKHYYYGGSYAMDYWGQGTSVTVSSTTTPAPRPPTPAPTIASQPLS | |
| | LRPEACEMYVAAAAFVLLFFVGCGVLLSKRKRKHGSGSGSGSMKLLSSI | |
| | EQACDICRLKKLKCSKEKPKCAKCLKNNWECRYSPKTKRSPLTRAHLTE | |
| | VESRLERLEQLFLLIFPREDLDMILKMDSLQDIKALLTGLFVQDNVNKD | |
| | AVTDRLASVETDMPLTLRQHRISATSSSEESSNKGQRQLTVSAAAGGSG | |
| | GSGGSDALDDFDLDMLGSDALDDEDLDMLGSDALDDEDLDMLGSDALDD | |
| | FDLDMLGSGSGSGSGSRVKFSRSADAPAYKQGQNQLYNELNLGRREEYD | |
| | VLDKRRGRDPEMGGKPRRKNPQEGLYNELQKDKMAEAYSEIGMKGERRR | |
| | GKGHDGLYQGLSTATKDTYDALHMQALPPRGSGSGSGSKRGRKKLLYIF | |
| | KQPFMRPVQTTQEEDGCSCRFPEEEEGGCEL | |
| FULL | MALPVTALLLPLALLLHAARPDYKDDDDKDIQMTQTTSSLSASLGDRVT | 21 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACFMYVAAAAFVLLFFVGCGVLLSKRKRKHGSGSGSGSKRGRKKLLY | |
| | IFKQPFMRPVQTTQEEDGCSCRFPEEEEGGCELGSGSGSGSMKLLSSIE | |
| | QACDICRLKKLKCSKEKPKCAKCLKNNWECRYSPKTKRSPLTRAHLTEV | |
| | ESRLERLEQLFLLIFPREDLDMILKMDSLQDIKALLTGLFVQDNVNKDA | |
| | VTDRLASVETDMPLTLRQHRISATSSSEESSNKGQRQLTVSAAAGGSGG | |
| | SGGSDALDDEDLDMLGSDALDDEDLDMLGSDALDDEDLDMLGSDALDDE | |
| | DLDMLGSGSGSGSGSRVKESRSADAPAYKQGQNQLYNELNLGRREEYDV | |
| | LDKRRGRDPEMGGKPRRKNPQEGLYNELQKDKMAEAYSEIGMKGERRRG | |
| | KGHDGLYQGLSTATKDTYDALHMQALPPR | |
| FULL | MALPVTALLLPLALLLHAARPDYKDDDDKDIQMTQTTSSLSASLGDRVT | 22 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACFMYVAAAAFVLLFFVGCGVLLSKRKRKHGSGSGSGSRVKFSRSAD | |
| | APAYKQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQ | |
| | ALPPRGSGSGSGSKRGRKKLLYIFKQPFMRPVQTTQEEDGCSCRFPEEE | |
| | EGGCELGSGSGSGSMKLLSSIEQACDICRLKKLKCSKEKPKCAKCLKNN | |
| | WECRYSPKTKRSPLTRAHLTEVESRLERLEQLFLLIFPREDLDMILKMD | |
| | SLQDIKALLTGLFVQDNVNKDAVTDRLASVETDMPLTLRQHRISATSSS | |
| | EESSNKGQRQLTVSAAAGGSGGSGGSDALDDFDLDMLGSDALDDEDLDM | |
| | LGSDALDDEDLDMLGSDALDDEDLDMLGS | |
| FULL | MALPVTALLLPLALLLHAARPDYKDDDDKDIQMTQTTSSLSASLGDRVT | 23 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACFMYVAAAAFVLLFFVGCGVLLSKRKRKHGSGSGSGSRVKFSRSAD | |
| | APAYKQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQ | |
| | ALPPRGSGSGSGSMKLLSSIEQACDICRLKKLKCSKEKPKCAKCLKNNW | |
| | ECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSE | |
| | ESSNKGQRQLTVSAAAGGSGGSGGSDALDDFDLDMLGSDALDDEDLDML | |
| | GSDALDDFDLDMLGSDALDDEDLDMLGSGSGSGSGSKRGRKKLLYIFKQ | |
| | PFMRPVQTTQEEDGCSCRFPEEEEGGCEL | |
| FULL | MALPVTALLLPLALLLHAARPDYKDDDDKDIQMTQTTSSLSASLGDRVT | 24 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACFMYVAAAAFVLLFFVGCGVLLSKRKRKHGSGSGSGSKRGRKKLLY | |
| | IFKQPFMRPVQTTQEEDGCSCRFPEEEEGGCELGSGSGSGSRVKESRSA | |
| | DAPAYKQGQNQLYNELNLGRREEYDVLDKRRGRDPEMGGKPRRKNPQEG | |
| | LYNELQKDKMAEAYSEIGMKGERRRGKGHDGLYQGLSTATKDTYDALHM | |
| | QALPPRGSGSGSGSMKLLSSIEQACDICRLKKLKCSKEKPKCAKCLKNN | |
| | WECRYSPKTKRSPLTRAHLTEVESRLERLEQLELLIFPREDLDMILKMD | |
| | SLQDIKALLTGLFVQDNVNKDAVTDRLASVETDMPLTLRQHRISATSSS | |
| | EESSNKGQRQLTVSAAAGGSGGSGGSDALDDEDLDMLGSDALDDEDLDM | |
| | LGSDALDDEDLDMLGSDALDDEDLDMLGS | |
| FULL | MALPVTALLLPLALLLHAARPDYKDDDDKDIQMTQTTSSLSASLGDRVT | 25 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSMKLLSSIEQ | |
| | ACDICRLKKLKCSKEKPKCAKCLKNNWECRYSPKTKRSPLTRAHLTEVE | |
| | SRLERLEQLFLLIFPREDLDMILKMDSLQDIKALLTGLFVQDNVNKDAV | |
| | TDRLASVETDMPLTLRQHRISATSSSEESSNKGQRQLTVSAAAGGSGGS | |
| | GGSDALDDFDLDMLGSDALDDEDLDMLGSDALDDEDLDMLGSDALDDED | |
| | LDMLGSGSGSGSGSKRGRKKLLYIFKQPFMRPVQTTQEEDGCSCREPEE | |
| | EEGGCELGSGSGSGSRVKFSRSADAPAYKQGQNQLYNELNLGRREEYDV | |
| | LDKRRGRDPEMGGKPRRKNPQEGLYNELQKDKMAEAYSEIGMKGERRRG | |
| | KGHDGLYQGLSTATKDTYDALHMQALPPR | |
| FULL | MALPVTALLLPLALLLHAARPDYKDDDDKDIQMTQTTSSLSASLGDRVT | 26 |
| SEQUENCE | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSMKLLSSIEQ | |
| | ACDICRLKKLKCSKEKPKCAKCLKNNWECRYSPKTKRSPLTRAHLTEVE | |
| | SRLERLEQLELLIFPREDLDMILKMDSLQDIKALLTGLFVQDNVNKDAV | |
| | TDRLASVETDMPLTLRQHRISATSSSEESSNKGQRQLTVSAAAGGSGGS | |
| | GGSDALDDFDLDMLGSDALDDEDLDMLGSDALDDEDLDMLGSDALDDED | |
| | LDMLGSGSGSGSGSRVKFSRSADAPAYKQGQNQLYNELNLGRREEYDVL | |
| | DKRRGRDPEMGGKPRRKNPQEGLYNELQKDKMAEAYSEIGMKGERRRGK | |
| | GHDGLYQGLSTATKDTYDALHMQALPPRGSGSGSGSKRGRKKLLYIFKQ | |
| | PFMRPVQTTQEEDGCSCRFPEEEEGGCEL | |
| FULL | MALPVTALLLPLALLLHAARPDYKDDDDKDIQMTQTTSSLSASLGDRVT | 27 |
| SEQUENCE | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSLLYIFKQPF | |
| | MRPVQTTQEEDGCSCRFPEEEEGGCELGSGSGSGSRVKFSRSADAPAYK | |
| | QGQNQLYNELNLGRREEYDVLDKRRGRDPEMGGKPRRKNPQEGLYNELQ | |
| | KDKMAEAYSEIGMKGERRRGKGHDGLYQGLSTATKDTYDALHMQALPPR | |
| | GSGSGSGSMKLLSSIEQACDICRLKKLKCSKEKPKCAKCLKNNWECRYS | |
| | PKTKRSPLTRAHLTEVESRLERLEQLELLIFPREDLDMILKMDSLQDIK | |
| | ALLTGLFVQDNVNKDAVTDRLASVETDMPLTLRQHRISATSSSEESSNK | |
| | GQRQLTVSAAAGGSGGSGGSDALDDEDLDMLGSDALDDEDLDMLGSDAL | |
| | DDFDLDMLGSDALDDEDLDMLGS | |
| FULL | MALPVTALLLPLALLLHAARPDYKDDDDKDIQMTQTTSSLSASLGDRVT | 28 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSPVQTTQEED | |
| | GCSCRFPEEEEGGCELGSGSGSGSRVKESRSADAPAYKQGQNQLYNELN | |
| | LGRREEYDVLDKRRGRDPEMGGKPRRKNPQEGLYNELQKDKMAEAYSEI | |
| | GMKGERRRGKGHDGLYQGLSTATKDTYDALHMQALPPRGSGSGSGSMKL | |
| | LSSIEQACDICRLKKLKCSKEKPKCAKCLKNNWECRYSPKTKRSPLTRA | |
| | HLTEVESRLERLEQLFLLIFPREDLDMILKMDSLQDIKALLTGLFVQDN | |
| | VNKDAVTDRLASVETDMPLTLRQHRISATSSSEESSNKGQRQLTVSAAA | |
| | GGSGGSGGSDALDDFDLDMLGSDALDDEDLDMLGSDALDDEDLDMLGSD | |
| | ALDDEDLDMLGS | |
| FULL | MALPVTALLLPLALLLHAARPDYKDDDDKDIQMTQTTSSLSASLGDRVT | 29 |
| SEQUENCE | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSPVQTTQEED | |
| | GCSCRFPEEEEGGCELPVQTTQEEDGCSCRFPEEEEGGCELGSGSGSGS | |
| | RVKFSRSADAPAYKQGQNQLYNELNLGRREEYDVLDKRRGRDPEMGGKP | |
| | RRKNPQEGLYNELQKDKMAEAYSEIGMKGERRRGKGHDGLYQGLSTATK | |
| | DTYDALHMQALPPRGSGSGSGSMKLLSSIEQACDICRLKKLKCSKEKPK | |
| | CAKCLKNNWECRYSPKTKRSPLTRAHLTEVESRLERLEQLELLIFPRED | |
| | LDMILKMDSLQDIKALLTGLFVQDNVNKDAVTDRLASVETDMPLTLRQH | |
| | RISATSSSEESSNKGQRQLTVSAAAGGSGGSGGSDALDDFDLDMLGSDA | |
| | LDDFDLDMLGSDALDDEDLDMLGSDALDDEDLDMLGS | |
| FULL | MALPVTALLLPLALLLHAARPDYKDDDDKDIQMTQTTSSLSASLGDRVT | 30 |
| SEQUENCE | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSPVQTTQEED | |
| | GCSCRFPEEEEGGCELPVQTTQEEDGCSCRFPEEEEGGCELPVQTTQEE | |
| | DGCSCRFPEEEEGGCELGSGSGSGSRVKFSRSADAPAYKQGQNQLYNEL | |
| | NLGRREEYDVLDKRRGRDPEMGGKPRRKNPQEGLYNELQKDKMAEAYSE | |
| | IGMKGERRRGKGHDGLYQGLSTATKDTYDALHMQALPPRGSGSGSGSMK | |
| | LLSSIEQACDICRLKKLKCSKEKPKCAKCLKNNWECRYSPKTKRSPLTR | |
| | AHLTEVESRLERLEQLFLLIFPREDLDMILKMDSLQDIKALLTGLFVQD | |
| | NVNKDAVTDRLASVETDMPLTLRQHRISATSSSEESSNKGQRQLTVSAA | |
| | AGGSGGSGGSDALDDEDLDMLGSDALDDEDLDMLGSDALDDEDLDMLGS | |
| | DALDDEDLDMLGS | |
| FULL | MALPVTALLLPLALLLHAARPDYKDDDDKDIQMTQTTSSLSASLGDRVT | 31 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSPVQTTQEED | |
| | GCSGSGSGSGSRVKFSRSADAPAYKQGQNQLYNELNLGRREEYDVLDKR | |
| | RGRDPEMGGKPRRKNPQEGLYNELQKDKMAEAYSEIGMKGERRRGKGHD | |
| | GLYQGLSTATKDTYDALHMQALPPRGSGSGSGSMKLLSSIEQACDICRL | |
| | KKLKCSKEKPKCAKCLKNNWECRYSPKTKRSPLTRAHLTEVESRLERLE | |
| | QLFLLIFPREDLDMILKMDSLQDIKALLTGLFVQDNVNKDAVTDRLASV | |
| | ETDMPLTLRQHRISATSSSEESSNKGQRQLTVSAAAGGSGGSGGSDALD | |
| | DFDLDMLGSDALDDEDLDMLGSDALDDEDLDMLGSDALDDEDLDMLGS | |
| FULL | MALPVTALLLPLALLLHAARPEQKLISEEDLDIQMTQTTSSLSASLGDR | 32 |
| SEQUENCE | VTISCRASQDISKYLNWYQQKPDGTVKLLIYHTSRLHSGVPSRESGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVELKMNSLQTDDTAI | |
| | YYCAKHYYYGGSYAMDYWGQGTSVTVSSTTTPAPRPPTPAPTIASQPLS | |
| | LRPEACFMYVAAAAFVLLFFVGCGVLLSKRKRKHGSGSGSGSRSKRSRL | |
| | LHSDYMNMTPRRPGPTRKHYQPYAPPRDFAAYRSGSGSGSGSMKLLSSI | |
| | EQACDICRLKKLKCSKEKPKCAKCLKNNWECRYSPKTKRSPLTRAHLTE | |
| | VESRLERLEQLFLLIFPREDLDMILKMDSLQDIKALLTGLFVQDNVNKD | |
| | AVTDRLASVETDMPLTLRQHRISATSSSEESSNKGQRQLTVSAAAGGSG | |
| | GSGGSDALDDFDLDMLGSDALDDEDLDMLGSDALDDEDLDMLGSDALDD | |
| | FDLDMLGSGSGSGSGSRVKESRSADAPAYKQGQNQLYNELNLGRREEYD | |
| | VLDKRRGRDPEMGGKPRRKNPQEGLYNELQKDKMAEAYSEIGMKGERRR | |
| | GKGHDGLYQGLSTATKDTYDALHMQALPPR | |
| FULL | MALPVTALLLPLALLLHAARPEQKLISEEDLDIQMTQTTSSLSASLGDR | 34 |
| SEQUENCE | VTISCRASQDISKYLNWYQQKPDGTVKLLIYHTSRLHSGVPSRFSGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVFLKMNSLQTDDTAI | |
| | YYCAKHYYYGGSYAMDYWGQGTSVTVSSTTTPAPRPPTPAPTIASQPLS | |
| | LRPEACEMYVAAAAFVLLFFVGCGVLLSKRKRKHGSGSGSGSRVKFSRS | |
| | ADAPAYKQGQNQLYNELNLGRREEYDVLDKRRGRDPEMGGKPRRKNPQE | |
| | GLYNELQKDKMAEAYSEIGMKGERRRGKGHDGLYQGLSTATKDTYDALH | |
| | MQALPPRGSGSGSGSRSKRSRLLHSDYMNMTPRRPGPTRKHYQPYAPPR | |
| | DFAAYRSGSGSGSGSMKLLSSIEQACDICRLKKLKCSKEKPKCAKCLKN | |
| | NWECRYSPKTKRSPLTRAHLTEVESRLERLEQLELLIFPREDLDMILKM | |
| | DSLQDIKALLTGLFVQDNVNKDAVTDRLASVETDMPLTLRQHRISATSS | |
| | SEESSNKGQRQLTVSAAAGGSGGSGGSDALDDEDLDMLGSDALDDEDLD | |
| | MLGSDALDDEDLDMLGSDALDDEDLDMLGS | |
| FULL | MALPVTALLLPLALLLHAARPEQKLISEEDLDIQMTQTTSSLSASLGDR | 35 |
| SEQUENCE | VTISCRASQDISKYLNWYQQKPDGTVKLLIYHTSRLHSGVPSRESGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVFLKMNSLQTDDTAI | |
| | YYCAKHYYYGGSYAMDYWGQGTSVTVSSTTTPAPRPPTPAPTIASQPLS | |
| | LRPEACEMYVAAAAFVLLFFVGCGVLLSKRKRKHGSGSGSGSRVKESRS | |
| | ADAPAYKQGQNQLYNELNLGRREEYDVLDKRRGRDPEMGGKPRRKNPQE | |
| | GLYNELQKDKMAEAYSEIGMKGERRRGKGHDGLYQGLSTATKDTYDALH | |
| | MQALPPRGSGSGSGSMKLLSSIEQACDICRLKKLKCSKEKPKCAKCLKN | |
| | NWECRYSPKTKRSPLTRAHLTEVESRLERLEQLFLLIFPREDLDMILKM | |
| | DSLQDIKALLTGLFVQDNVNKDAVTDRLASVETDMPLTLRQHRISATSS | |
| | SEESSNKGQRQLTVSAAAGGSGGSGGSDALDDEDLDMLGSDALDDEDLD | |
| | MLGSDALDDEDLDMLGSDALDDEDLDMLGSGSGSGSGSRSKRSRLLHSD | |
| | YMNMTPRRPGPTRKHYQPYAPPRDFAAYRS | |
| FULL | MALPVTALLLPLALLLHAARPEQKLISEEDLDIQMTQTTSSLSASLGDR | 36 |
| SEQUENCE | VTISCRASQDISKYLNWYQQKPDGTVKLLIYHTSRLHSGVPSRESGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVFLKMNSLQTDDTAI | |
| | YYCAKHYYYGGSYAMDYWGQGTSVTVSSTTTPAPRPPTPAPTIASQPLS | |
| | LRPEACEMYVAAAAFVLLFFVGCGVLLSKRKRKHGSGSGSGSRSKRSRL | |
| | LHSDYMNMTPRRPGPTRKHYQPYAPPRDFAAYRSGSGSGSGSRVKESRS | |
| | ADAPAYKQGQNQLYNELNLGRREEYDVLDKRRGRDPEMGGKPRRKNPQE | |
| | GLYNELQKDKMAEAYSEIGMKGERRRGKGHDGLYQGLSTATKDTYDALH | |
| | MQALPPRGSGSGSGSMKLLSSIEQACDICRLKKLKCSKEKPKCAKCLKN | |
| | NWECRYSPKTKRSPLTRAHLTEVESRLERLEQLELLIFPREDLDMILKM | |
| | DSLQDIKALLTGLFVQDNVNKDAVTDRLASVETDMPLTLRQHRISATSS | |
| | SEESSNKGQRQLTVSAAAGGSGGSGGSDALDDEDLDMLGSDALDDEDLD | |
| | MLGSDALDDFDLDMLGSDALDDEDLDMLGS | |
| FULL | MALPVTALLLPLALLLHAARPEQKLISEEDLDIQMTQTTSSLSASLGDR | 37 |
| SEQUENCE | VTISCRASQDISKYLNWYQQKPDGTVKLLIYHTSRLHSGVPSRESGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVFLKMNSLQTDDTAI | |
| | YYCAKHYYYGGSYAMDYWGQGTSVTVSSTTTPAPRPPTPAPTIASQPLS | |
| | LRPEACEMYVAAAAFVLLFFVGCGVLLSKRKRKHGSGSGSGSMKLLSSI | |
| | EQACDICRLKKLKCSKEKPKCAKCLKNNWECRYSPKTKRSPLTRAHLTE | |
| | VESRLERLEQLFLLIFPREDLDMILKMDSLQDIKALLTGLFVQDNVNKD | |
| | AVTDRLASVETDMPLTLRQHRISATSSSEESSNKGQRQLTVSAAAGGSG | |
| | GSGGSDALDDEDLDMLGSDALDDEDLDMLGSDALDDEDLDMLGSDALDD | |
| | FDLDMLGSGSGSGSGSRSKRSRLLHSDYMNMTPRRPGPTRKHYQPYAPP | |
| | RDFAAYRSGSGSGSGSRVKESRSADAPAYKQGQNQLYNELNLGRREEYD | |
| | VLDKRRGRDPEMGGKPRRKNPQEGLYNELQKDKMAEAYSEIGMKGERRR | |
| | GKGHDGLYQGLSTATKDTYDALHMQALPPR | |
| FULL | MALPVTALLLPLALLLHAARPEQKLISEEDLDIQMTQTTSSLSASLGDR | 38 |
| SEQUENCE | VTISCRASQDISKYLNWYQQKPDGTVKLLIYHTSRLHSGVPSRFSGSGS | |
| | GTDYSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQPPRK | |
| | GLEWLGVIWGSETTYYNSALKSRLTIIKDNSKSQVELKMNSLQTDDTAI | |
| | YYCAKHYYYGGSYAMDYWGQGTSVTVSSTTTPAPRPPTPAPTIASQPLS | |
| | LRPEACEMYVAAAAFVLLFFVGCGVLLSKRKRKHGSGSGSGSMKLLSSI | |
| | EQACDICRLKKLKCSKEKPKCAKCLKNNWECRYSPKTKRSPLTRAHLTE | |
| | VESRLERLEQLFLLIFPREDLDMILKMDSLQDIKALLTGLFVQDNVNKD | |
| | AVTDRLASVETDMPLTLRQHRISATSSSEESSNKGQRQLTVSAAAGGSG | |
| | GSGGSDALDDFDLDMLGSDALDDEDLDMLGSDALDDEDLDMLGSDALDD | |
| | FDLDMLGSGSGSGSGSRVKFSRSADAPAYKQGQNQLYNELNLGRREEYD | |
| | VLDKRRGRDPEMGGKPRRKNPQEGLYNELQKDKMAEAYSEIGMKGERRR | |
| | GKGHDGLYQGLSTATKDTYDALHMQALPPRGSGSGSGSRSKRSRLLHSD | |
| | YMNMTPRRPGPTRKHYQPYAPPRDFAAYRS | |
| FULL | MALPVTALLLPLALLLHAARPDYKDDDDKDIQMTQTTSSLSASLGDRVT | 39 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSRSKRSRLLH | |
| | SDYMNMTPRRPGPTRKHYQPYAPPRDFAAYRSGSGSGSGSMKLLSSIEQ | |
| | ACDICRLKKLKCSKEKPKCAKCLKNNWECRYSPKTKRSPLTRAHLTEVE | |
| | SRLERLEQLFLLIFPREDLDMILKMDSLQDIKALLTGLFVQDNVNKDAV | |
| | TDRLASVETDMPLTLRQHRISATSSSEESSNKGQRQLTVSAAAGGSGGS | |
| | GGSDALDDFDLDMLGSDALDDEDLDMLGSDALDDEDLDMLGSDALDDED | |
| | LDMLGSGSGSGSGSRVKFSRSADAPAYKQGQNQLYNELNLGRREEYDVL | |
| | DKRRGRDPEMGGKPRRKNPQEGLYNELQKDKMAEAYSEIGMKGERRRGK | |
| | GHDGLYQGLSTATKDTYDALHMQALPPR | |
| FULL | MALPVTALLLPLALLLHAARPDYKDDDDKDIQMTQTTSSLSASLGDRVT | 40 |
| SEQUENCE | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSRVKESRSAD | |
| | APAYKQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQ | |
| | ALPPRGSGSGSGSRSKRSRLLHSDYMNMTPRRPGPTRKHYQPYAPPRDE | |
| | AAYRSGSGSGSGSMKLLSSIEQACDICRLKKLKCSKEKPKCAKCLKNNW | |
| | ECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSE | |
| | ESSNKGQRQLTVSAAAGGSGGSGGSDALDDFDLDMLGSDALDDEDLDML | |
| | GSDALDDEDLDMLGSDALDDEDLDMLGS | |
| FULL | MALPVTALLLPLALLLHAARPDYKDDDDKDIQMTQTTSSLSASLGDRVT | 41 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSRVKESRSAD | |
| | APAYKQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQ | |
| | ALPPRGSGSGSGSMKLLSSIEQACDICRLKKLKCSKEKPKCAKCLKNNW | |
| | ECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSE | |
| | ESSNKGQRQLTVSAAAGGSGGSGGSDALDDEDLDMLGSDALDDEDLDML | |
| | GSDALDDFDLDMLGSDALDDFDLDMLGSGSGSGSGSRSKRSRLLHSDYM | |
| | NMTPRRPGPTRKHYQPYAPPRDFAAYRS | |
| FULL | MALPVTALLLPLALLLHAARPDYKDDDDKDIQMTQTTSSLSASLGDRVT | 42 |
| SEQUENCE | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSRSKRSRLLH | |
| | SDYMNMTPRRPGPTRKHYQPYAPPRDFAAYRSGSGSGSGSRVKFSRSAD | |
| | APAYKQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQ | |
| | ALPPRGSGSGSGSMKLLSSIEQACDICRLKKLKCSKEKPKCAKCLKNNW | |
| | ECRYSPKTKRSPLTRAHLTEVESRLERLEQLFLLIFPREDLDMILKMDS | |
| | LQDIKALLTGLFVQDNVNKDAVTDRLASVETDMPLTLRQHRISATSSSE | |
| | ESSNKGQRQLTVSAAAGGSGGSGGSDALDDEDLDMLGSDALDDEDLDML | |
| | GSDALDDFDLDMLGSDALDDEDLDMLGS | |
| FULL | MALPVTALLLPLALLLHAARPDYKDDDDKDIQMTQTTSSLSASLGDRVT | 43 |
| SEQUENCE | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSMKLLSSIEQ | |
| | ACDICRLKKLKCSKEKPKCAKCLKNNWECRYSPKTKRSPLTRAHLTEVE | |
| | SRLERLEQLFLLIFPREDLDMILKMDSLQDIKALLTGLFVQDNVNKDAV | |
| | TDRLASVETDMPLTLRQHRISATSSSEESSNKGQRQLTVSAAAGGSGGS | |
| | GGSDALDDFDLDMLGSDALDDFDLDMLGSDALDDEDLDMLGSDALDDED | |
| | LDMLGSGSGSGSGSRSKRSRLLHSDYMNMTPRRPGPTRKHYQPYAPPRD | |
| | FAAYRSGSGSGSGSRVKFSRSADAPAYKQGQNQLYNELNLGRREEYDVL | |
| | DKRRGRDPEMGGKPRRKNPQEGLYNELQKDKMAEAYSEIGMKGERRRGK | |
| | GHDGLYQGLSTATKDTYDALHMQALPPR | |
| FULL | MALPVTALLLPLALLLHAARPDYKDDDDKDIQMTQTTSSLSASLGDRVT | 44 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSMKLLSSIEQ | |
| | ACDICRLKKLKCSKEKPKCAKCLKNNWECRYSPKTKRSPLTRAHLTEVE | |
| | SRLERLEQLELLIFPREDLDMILKMDSLQDIKALLTGLFVQDNVNKDAV | |
| | TDRLASVETDMPLTLRQHRISATSSSEESSNKGQRQLTVSAAAGGSGGS | |
| | GGSDALDDFDLDMLGSDALDDEDLDMLGSDALDDEDLDMLGSDALDDED | |
| | LDMLGSGSGSGSGSRVKFSRSADAPAYKQGQNQLYNELNLGRREEYDVL | |
| | DKRRGRDPEMGGKPRRKNPQEGLYNELQKDKMAEAYSEIGMKGERRRGK | |
| | GHDGLYQGLSTATKDTYDALHMQALPPRGSGSGSGSRSKRSRLLHSDYM | |
| | NMTPRRPGPTRKHYQPYAPPRDEAAYRS | |
| ECD | MALPVTALLLPLALLLHAARPDYKDDDDKQVQLVQSGAEVKKPGASVKV | 45 |
| | SCKASGYSFPDYYINWVRQAPGQGLEWMGWIYFASGNSEYNQKFTGRVT | |
| | MTRDTSINTAYMELSSLTSEDTAVYFCASLYDYDWYFDVWGQGTMVTVS | |
| | SGGGGSGGGGSGGGGSDIVMTQTPLSLSVTPGQPASISCKSSQSLVHSN | |
| | GNTYLHWYLQKPGQSPQLLIYKVSNRFSGVPDRESGSGSGTDFTLKISR | |
| | VEAEDVGIYYCSQSSIYPWTFGQGTKLEIK | |
| ECD | MALPVTALLLPLALLLHAARPDYKDDDDKQVQLQQSGGGLVKPGGSLRL | 46 |
| | SCAASGFTESSYAMHWVRQAPGKGLEWVAVISYDGSNKYYADSVKGRFT | |
| | ISRDNSKNTLYLQMDSLRAEDTAVYYCAKEGDSSRWSYDLWGRGTLVTV | |
| | SSGGGGSGGGGSGGGGSQSALTQPASVSGSPGQSITISCTGTSSDVGGY | |
| | NYVSWYQQHPGKAPKVMIYDVTNRPSGVSNRFSGSKSGNTASLTISGLQ | |
| | AEDEADYYCSSYTIASTLVVFGGGTKLTVL | |
| FULL | MALPVTALLLPLALLLHAARPDYKDDDDKQVQLVQSGAEVKKPGASVKV | 47 |
| SEQUENCE | SCKASGYSFPDYYINWVRQAPGQGLEWMGWIYFASGNSEYNQKFTGRVT | |
| | MTRDTSINTAYMELSSLTSEDTAVYFCASLYDYDWYFDVWGQGTMVTVS | |
| | SGGGGSGGGGSGGGGSDIVMTQTPLSLSVTPGQPASISCKSSQSLVHSN | |
| | GNTYLHWYLQKPGQSPQLLIYKVSNRFSGVPDRESGSGSGTDFTLKISR | |
| | VEAEDVGIYYCSQSSIYPWTFGQGTKLEIKTTTPAPRPPTPAPTIASQP | |
| | LSLRPEACEMYVAAAAFVLLFFVGCGVLLSKRKRKHGSGSGSGSPVQTT | |
| | QEEDGCSCRFPEEEEGGCELGSGSGSGSRVKESRSADAPAYKQGQNQLY | |
| | NELNLGRREEYDVLDKRRGRDPEMGGKPRRKNPQEGLYNELQKDKMAEA | |
| | YSEIGMKGERRRGKGHDGLYQGLSTATKDTYDALHMQALPPRGSGSGSG | |
| | SMKLLSSIEQACDICRLKKLKCSKEKPKCAKCLKNNWECRYSPKTKRSP | |
| | LTRAHLTEVESRLERLEQLELLIFPREDLDMILKMDSLQDIKALLTGLE | |
| | VQDNVNKDAVTDRLASVETDMPLTLRQHRISATSSSEESSNKGQRQLTV | |
| | SAAAGGSGGSGGSDALDDEDLDMLGSDALDDEDLDMLGSDALDDEDLDM | |
| | LGSDALDDFDLDMLGS | |
| FULL | MALPVTALLLPLALLLHAARPDYKDDDDKQVQLQQSGGGLVKPGGSLRL | 48 |
| SEQUENCE | SCAASGFTESSYAMHWVRQAPGKGLEWVAVISYDGSNKYYADSVKGRFT | |
| | ISRDNSKNTLYLQMDSLRAEDTAVYYCAKEGDSSRWSYDLWGRGTLVTV | |
| | SSGGGGSGGGGSGGGGSQSALTQPASVSGSPGQSITISCTGTSSDVGGY | |
| | NYVSWYQQHPGKAPKVMIYDVTNRPSGVSNRFSGSKSGNTASLTISGLQ | |
| | AEDEADYYCSSYTIASTLVVFGGGTKLTVLTTTPAPRPPTPAPTIASQP | |
| | LSLRPEACEMYVAAAAFVLLFFVGCGVLLSKRKRKHGSGSGSGSPVQTT | |
| | QEEDGCSCRFPEEEEGGCELGSGSGSGSRVKFSRSADAPAYKQGQNQLY | |
| | NELNLGRREEYDVLDKRRGRDPEMGGKPRRKNPQEGLYNELQKDKMAEA | |
| | YSEIGMKGERRRGKGHDGLYQGLSTATKDTYDALHMQALPPRGSGSGSG | |
| | SMKLLSSIEQACDICRLKKLKCSKEKPKCAKCLKNNWECRYSPKTKRSP | |
| | LTRAHLTEVESRLERLEQLFLLIFPREDLDMILKMDSLQDIKALLTGLF | |
| | VQDNVNKDAVTDRLASVETDMPLTLRQHRISATSSSEESSNKGQRQLTV | |
| | SAAAGGSGGSGGSDALDDFDLDMLGSDALDDEDLDMLGSDALDDEDLDM | |
| | LGSDALDDEDLDMLGS | |
| ICD | LLHSDYMNMTPRRPGPTRKHYQPYAPPRDFAAYRS | 49 |
| ICD | SDYMNMTPRRPGPTRKHYQPYAPPRDFAAYRS | 50 |
| ICD | RSKRSRLLHSDYMNMGPTRKHYQPYAPPRDFAAYRS | 51 |
| ICD | SDYMNMGPTRKHYQPYAPPRDFAAYRS | 52 |
| ICD | GPTRKHYQPYAPPRDFAAYRS | 53 |
| FULL | MALPVTALLLPLALLLHAARPDYKDDDDKDIQMTQTTSSLSASLGDRVT | 54 |
| SEQUENCE | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSLLHSDYMNM | |
| | TPRRPGPTRKHYQPYAPPRDFAAYRSGSGSGSGSRVKFSRSADAPAYKQ | |
| | GQNQLYNELNLGRREEYDVLDKRRGRDPEMGGKPRRKNPQEGLYNELQK | |
| | DKMAEAYSEIGMKGERRRGKGHDGLYQGLSTATKDTYDALHMQALPPRG | |
| | SGSGSGSMKLLSSIEQACDICRLKKLKCSKEKPKCAKCLKNNWECRYSP | |
| | KTKRSPLTRAHLTEVESRLERLEQLFLLIFPREDLDMILKMDSLQDIKA | |
| | LLTGLFVQDNVNKDAVTDRLASVETDMPLTLRQHRISATSSSEESSNKG | |
| | QRQLTVSAAAGGSGGSGGSDALDDFDLDMLGSDALDDEDLDMLGSDALD | |
| | DFDLDMLGSDALDDEDLDMLGS | |
| FULL | MALPVTALLLPLALLLHAARPDYKDDDDKDIQMTQTTSSLSASLGDRVT | 55 |
| SEQUENCE | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSSDYMNMTPR | |
| | RPGPTRKHYQPYAPPRDFAAYRSGSGSGSGSRVKESRSADAPAYKQGQN | |
| | QLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPRGSGS | |
| | GSGSMKLLSSIEQACDICRLKKLKCSKEKPKCAKCLKNNWECRYSPKTK | |
| | RSPLTRAHLTEVESRLERLEQLFLLIFPREDLDMILKMDSLQDIKALLT | |
| | GLFVQDNVNKDAVTDRLASVETDMPLTLRQHRISATSSSEESSNKGQRQ | |
| | LTVSAAAGGSGGSGGSDALDDFDLDMLGSDALDDEDLDMLGSDALDDED | |
| | LDMLGSDALDDEDLDMLGS | |
| FULL | MALPVTALLLPLALLLHAARPDYKDDDDKDIQMTQTTSSLSASLGDRVT | 56 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACEMYVAAAAFVLLEFVGCGVLLSKRKRKHGSGSGSGSRSKRSRLLH | |
| | SDYMNMGPTRKHYQPYAPPRDFAAYRSGSGSGSGSRVKFSRSADAPAYK | |
| | QGQNQLYNELNLGRREEYDVLDKRRGRDPEMGGKPRRKNPQEGLYNELQ | |
| | KDKMAEAYSEIGMKGERRRGKGHDGLYQGLSTATKDTYDALHMQALPPR | |
| | GSGSGSGSMKLLSSIEQACDICRLKKLKCSKEKPKCAKCLKNNWECRYS | |
| | PKTKRSPLTRAHLTEVESRLERLEQLFLLIFPREDLDMILKMDSLQDIK | |
| | ALLTGLFVQDNVNKDAVTDRLASVETDMPLTLRQHRISATSSSEESSNK | |
| | GQRQLTVSAAAGGSGGSGGSDALDDFDLDMLGSDALDDEDLDMLGSDAL | |
| | DDFDLDMLGSDALDDEDLDMLGS | |
| FULL | MALPVTALLLPLALLLHAARPDYKDDDDKDIQMTQTTSSLSASLGDRVT | 57 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACFMYVAAAAFVLLFFVGCGVLLSKRKRKHGSGSGSGSSDYMNMGPT | |
| | RKHYQPYAPPRDFAAYRSGSGSGSGSRVKESRSADAPAYKQGQNQLYNE | |
| | LNLGRREEYDVLDKRRGRDPEMGGKPRRKNPQEGLYNELQKDKMAEAYS | |
| | EIGMKGERRRGKGHDGLYQGLSTATKDTYDALHMQALPPRGSGSGSGSM | |
| | KLLSSIEQACDICRLKKLKCSKEKPKCAKCLKNNWECRYSPKTKRSPLT | |
| | RAHLTEVESRLERLEQLFLLIFPREDLDMILKMDSLQDIKALLTGLFVQ | |
| | DNVNKDAVTDRLASVETDMPLTLRQHRISATSSSEESSNKGQRQLTVSA | |
| | AAGGSGGSGGSDALDDEDLDMLGSDALDDEDLDMLGSDALDDEDLDMLG | |
| | SDALDDEDLDMLGS | |
| FULL | MALPVTALLLPLALLLHAARPDYKDDDDKDIQMTQTTSSLSASLGDRVT | 58 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSGPTRKHYQP | |
| | YAPPRDFAAYRSGSGSGSGSRVKFSRSADAPAYKQGQNQLYNELNLGRR | |
| | EEYDVLDKRRGRDPEMGGKPRRKNPQEGLYNELQKDKMAEAYSEIGMKG | |
| | ERRRGKGHDGLYQGLSTATKDTYDALHMQALPPRGSGSGSGSMKLLSSI | |
| | EQACDICRLKKLKCSKEKPKCAKCLKNNWECRYSPKTKRSPLTRAHLTE | |
| | VESRLERLEQLELLIFPREDLDMILKMDSLQDIKALLTGLFVQDNVNKD | |
| | AVTDRLASVETDMPLTLRQHRISATSSSEESSNKGQRQLTVSAAAGGSG | |
| | GSGGSDALDDFDLDMLGSDALDDEDLDMLGSDALDDEDLDMLGSDALDD | |
| | FDLDMLGS | |
| ICD | PVQTTQEEDGCSCRFPEEEEGGCELPYAP | 59 |
| ICD | PVQTTQEEDGCSCRFPEEEEGGCELYMEM | 60 |
| ICD | PVQTTQEEDGCSCRFPEEEEGGCELYMEMTPRRP | 61 |
| ICD | PVQTTQEEDGCSCRFPEEEEGGCELAAYRS | 62 |
| FULL | MALPVTALLLPLALLLHAARPDYKDDDDKDIQMTQTTSSLSASLGDRVT | 63 |
| SEQUENCE | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACFMYVAAAAFVLLFFVGCGVLLSKRKRKHGSGSGSGSPVQTTQEED | |
| | GCSCRFPEEEEGGCELPYAPGSGSGSGSRVKESRSADAPAYKQGQNQLY | |
| | NELNLGRREEYDVLDKRRGRDPEMGGKPRRKNPQEGLYNELQKDKMAEA | |
| | YSEIGMKGERRRGKGHDGLYQGLSTATKDTYDALHMQALPPRGSGSGSG | |
| | SMKLLSSIEQACDICRLKKLKCSKEKPKCAKCLKNNWECRYSPKTKRSP | |
| | LTRAHLTEVESRLERLEQLELLIFPREDLDMILKMDSLQDIKALLTGLF | |
| | VQDNVNKDAVTDRLASVETDMPLTLRQHRISATSSSEESSNKGQRQLTV | |
| | SAAAGGSGGSGGSDALDDFDLDMLGSDALDDEDLDMLGSDALDDEDLDM | |
| | LGSDALDDFDLDMLGS | |
| FULL | MALPVTALLLPLALLLHAARPDYKDDDDKDIQMTQTTSSLSASLGDRVT | 64 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACFMYVAAAAFVLLFFVGCGVLLSKRKRKHGSGSGSGSPVQTTQEED | |
| | GCSCRFPEEEEGGCELYMFMGSGSGSGSRVKESRSADAPAYKQGQNQLY | |
| | NELNLGRREEYDVLDKRRGRDPEMGGKPRRKNPQEGLYNELQKDKMAEA | |
| | YSEIGMKGERRRGKGHDGLYQGLSTATKDTYDALHMQALPPRGSGSGSG | |
| | SMKLLSSIEQACDICRLKKLKCSKEKPKCAKCLKNNWECRYSPKTKRSP | |
| | LTRAHLTEVESRLERLEQLFLLIFPREDLDMILKMDSLQDIKALLTGLF | |
| | VQDNVNKDAVTDRLASVETDMPLTLRQHRISATSSSEESSNKGQRQLTV | |
| | SAAAGGSGGSGGSDALDDEDLDMLGSDALDDEDLDMLGSDALDDEDLDM | |
| | LGSDALDDFDLDMLGS | |
| FULL | MALPVTALLLPLALLLHAARPDYKDDDDKDIQMTQTTSSLSASLGDRVT | 65 |
| SEQUENCE | ISCRASQDISKYLNWYQQKPDGTVKLLIYHTSRLHSGVPSRFSGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVELKMNSLQTDDTAIYY | |
| | CAKHYYYGGSYAMDYWGQGTSVTVSSTTTPAPRPPTPAPTIASQPLSLR | |
| | PEACEMYVAAAAFVLLFFVGCGVLLSKRKRKHGSGSGSGSPVQTTQEED | |
| | GCSCRFPEEEEGGCELYMFMTPRRPGSGSGSGSRVKFSRSADAPAYKQG | |
| | QNQLYNELNLGRREEYDVLDKRRGRDPEMGGKPRRKNPQEGLYNELQKD | |
| | KMAEAYSEIGMKGERRRGKGHDGLYQGLSTATKDTYDALHMQALPPRGS | |
| | GSGSGSMKLLSSIEQACDICRLKKLKCSKEKPKCAKCLKNNWECRYSPK | |
| | TKRSPLTRAHLTEVESRLERLEQLELLIFPREDLDMILKMDSLQDIKAL | |
| | LTGLFVQDNVNKDAVTDRLASVETDMPLTLRQHRISATSSSEESSNKGQ | |
| | RQLTVSAAAGGSGGSGGSDALDDFDLDMLGSDALDDFDLDMLGSDALDD | |
| | FDLDMLGSDALDDEDLDMLGS | |
| FULL | MALPVTALLLPLALLLHAARPDYKDDDDKDIQMTQTTSSLSASLGDRVT | 66 |
| SEQUENCE | ISCRASQDISKYLNWYQQKPDGTVKLLIYHTSRLHSGVPSRESGSGSGT | |
| | DYSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKGL | |
| | EWLGVIWGSETTYYNSALKSRLTIIKDNSKSQVFLKMNSLQTDDTAIYY | |
| | CAKHYYYGGSYAMDYWGQGTSVTVSSTTTPAPRPPTPAPTIASQPLSLR | |
| | PEACFMYVAAAAFVLLFFVGCGVLLSKRKRKHGSGSGSGSPVQTTQEED | |
| | GCSCRFPEEEEGGCELAAYRSGSGSGSGSRVKESRSADAPAYKQGQNQL | |
| | YNELNLGRREEYDVLDKRRGRDPEMGGKPRRKNPQEGLYNELQKDKMAE | |
| | AYSEIGMKGERRRGKGHDGLYQGLSTATKDTYDALHMQALPPRGSGSGS | |
| | GSMKLLSSIEQACDICRLKKLKCSKEKPKCAKCLKNNWECRYSPKTKRS | |
| | PLTRAHLTEVESRLERLEQLFLLIFPREDLDMILKMDSLQDIKALLTGL | |
| | FVQDNVNKDAVTDRLASVETDMPLTLRQHRISATSSSEESSNKGQRQLT | |
| | VSAAAGGSGGSGGSDALDDEDLDMLGSDALDDEDLDMLGSDALDDEDLD | |
| | MLGSDALDDEDLDMLGS | |
| FULL | MALPVTALLLPLALLLHAARPDYKDDDDKQVQLVQSGAEVKKPGASVKV | 67 |
| SEQUENCE | SCKASGYSFPDYYINWVRQAPGQGLEWMGWIYFASGNSEYNQKFTGRVT | |
| | MTRDTSINTAYMELSSLTSEDTAVYFCASLYDYDWYFDVWGQGTMVTVS | |
| | SGGGGSGGGGSGGGGSDIVMTQTPLSLSVTPGQPASISCKSSQSLVHSN | |
| | GNTYLHWYLQKPGQSPQLLIYKVSNRFSGVPDRESGSGSGTDETLKISR | |
| | VEAEDVGIYYCSQSSIYPWTFGQGTKLEIKTTTPAPRPPTPAPTIASQP | |
| | LSLRPEACEMYVAAAAFVLLFFVGCGVLLSKRKRKHGSGSGSGSPVQTT | |
| | QEEDGCSCRFPEEEEGGCELPYAPGSGSGSGSRVKFSRSADAPAYKQGQ | |
| | NQLYNELNLGRREEYDVLDKRRGRDPEMGGKPRRKNPQEGLYNELQKDK | |
| | MAEAYSEIGMKGERRRGKGHDGLYQGLSTATKDTYDALHMQALPPRGSG | |
| | SGSGSMKLLSSIEQACDICRLKKLKCSKEKPKCAKCLKNNWECRYSPKT | |
| | KRSPLTRAHLTEVESRLERLEQLFLLIFPREDLDMILKMDSLQDIKALL | |
| | TGLFVQDNVNKDAVTDRLASVETDMPLTLRQHRISATSSSEESSNKGQR | |
| | QLTVSAAAGGSGGSGGSDALDDEDLDMLGSDALDDEDLDMLGSDALDDE | |
| | DLDMLGSDALDDEDLDMLGS | |
| FULL | MALPVTALLLPLALLLHAARPDYKDDDDKQVQLQQSGGGLVKPGGSLRL | 68 |
| SEQUENCE | SCAASGFTFSSYAMHWVRQAPGKGLEWVAVISYDGSNKYYADSVKGRET | |
| | ISRDNSKNTLYLQMDSLRAEDTAVYYCAKEGDSSRWSYDLWGRGTLVTV | |
| | SSGGGGSGGGGSGGGGSQSALTQPASVSGSPGQSITISCTGTSSDVGGY | |
| | NYVSWYQQHPGKAPKVMIYDVTNRPSGVSNRFSGSKSGNTASLTISGLQ | |
| | AEDEADYYCSSYTIASTLVVFGGGTKLTVLTTTPAPRPPTPAPTIASQP | |
| | LSLRPEACEMYVAAAAFVLLFFVGCGVLLSKRKRKHGSGSGSGSPVQTT | |
| | QEEDGCSCRFPEEEEGGCELPYAPGSGSGSGSRVKESRSADAPAYKQGQ | |
| | NQLYNELNLGRREEYDVLDKRRGRDPEMGGKPRRKNPQEGLYNELQKDK | |
| | MAEAYSEIGMKGERRRGKGHDGLYQGLSTATKDTYDALHMQALPPRGSG | |
| | SGSGSMKLLSSIEQACDICRLKKLKCSKEKPKCAKCLKNNWECRYSPKT | |
| | KRSPLTRAHLTEVESRLERLEQLELLIFPREDLDMILKMDSLQDIKALL | |
| | TGLFVQDNVNKDAVTDRLASVETDMPLTLRQHRISATSSSEESSNKGQR | |
| | QLTVSAAAGGSGGSGGSDALDDFDLDMLGSDALDDEDLDMLGSDALDDE | |
| | DLDMLGSDALDDEDLDMLGS | |
| CD8α signal | MALPVTALLLPLALLLHAARP | 69 |
| peptide | | |
| flag-tag | DYKDDDDK | 70 |
| Gal4 DNA | GGAGCACTGTCCTCCGAACG | 71 |
| binding domain | | |
| target sequence | | |

What is claimed is:

1. A chimeric receptor comprising, from N-terminus to C-terminus:
a) an extracellular ligand-binding domain having a binding affinity for a selected ligand;
b) a linking polypeptide;
c) a transmembrane domain (TMD) comprising one or more ligand-inducible proteolytic cleavage sites; and
d) an intracellular domain (ICD) comprising, in any order:
(i) an intracellular signaling domain (SD) comprising at least one costimulatory domain derived from a signaling molecule and an activation domain, and
(ii) a transcriptional regulator, and
wherein binding of the selected ligand to the extracellular ligand-binding domain induces cleavage at a ligand-inducible proteolytic cleavage site disposed between the ICD and the linking polypeptide,
wherein binding of the selected ligand to the extracellular ligand-binding domain induces proximal signaling cascades through the intracellular SD, and
wherein the chimeric receptor does not comprise a LIN-12-Notch repeat (LNR) and/or a heterodimerization domain (HD) of a Notch receptor.

2. The chimeric receptor of claim 1, wherein the extracellular domain comprises an antigen-binding moiety capable of binding to a ligand on the surface of a cell.

3. The chimeric receptor of claim 2, wherein the cell is a pathogenic cell.

4. The chimeric receptor of claim 3, wherein the cell is a human cell.

5. The chimeric receptor of claim 4, wherein the human cell is a tumor cell.

6. The chimeric receptor of claim 4, wherein the human cell is a terminally differentiated cell.

7. The chimeric receptor of any one of the preceding claims, wherein the ligand comprises a protein or a carbohydrate.

8. The chimeric receptor of any one of the preceding claims, wherein the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B-cell maturation antigen (BCMA), green fluorescent protein (GFP), blue fluorescent protein (BFP) enhanced green fluorescent protein (EGFP), and signal regulatory protein α (SIRPα).

9. The chimeric receptor of any one of the preceding claims, wherein the ligand is selected from cell surface receptors, adhesion proteins, integrins, mucins, lectins, tumor-associated antigens, and tumor-specific antigens.

10. The chimeric receptor of any one of the preceding claims, wherein the ligand is a tumor-associated antigen or a tumor-specific antigen.

11. The chimeric receptor of any one of the preceding claims, wherein the extracellular ligand-binding domain comprises the ligand-binding portion of a receptor.

12. The chimeric receptor of any one of the preceding claims, wherein the antigen-binding moiety is selected from the group consisting of an antibody, a nanobody, a diabody, a triabody, a minibody, an F(ab′)2 fragment, an F(ab)v fragment, a single chain variable fragment (scFv), a single domain antibody (sdAb), and a functional fragment thereof.

13. The chimeric receptor of claim 12, wherein the antigen-binding moiety comprises an scFv.

14. The chimeric receptor of any one of the preceding claims, wherein the antigen-binding moiety specifically binds to a tumor-associated antigen selected from the group consisting of CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD 117), MUC1, NCAM, PAP, PDGFR-0, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl.

15. The chimeric receptor of claim 14, wherein the tumor-associated antigen is CD19, BCMA, CEA, HER2, MUC1, CD20, ALPPL2, SIRPα, or EGFR.

16. The chimeric receptor of claim 15, wherein the tumor-associated antigen is CD19, BCMA, HER2, or ALPPL2.

17. The chimeric receptor of any one of the preceding claims, wherein the linking polypeptide comprises a hinge domain.

18. The chimeric receptor of claim 17, wherein the hinge domain is capable of promoting oligomer formation of the chimeric polypeptide via intermolecular disulfide bonding.

19. The chimeric receptor of claim 17, wherein the hinge domain is derived from a CD8a hinge domain, a CD28 hinge domain, a CD152 hinge domain, a PD-1 hinge domain, a CTLA4 hinge domain, an OX40 hinge domain, an IgG1 hinge domain, an IgG2 hinge domain, an IgG3 hinge domain, and an IgG4 hinge domain, or a functional variant of any thereof.

20. The chimeric receptor of any one of the preceding claims, wherein the linking polypeptide is derived from the group selected from: a CD8a hinge domain or a functional variant thereof, a CD28 hinge domain or a functional variant thereof, OX40 hinge domain or a functional variant thereof, and an IgG4 hinge domain or a functional variant thereof.

21. The chimeric receptor of any one of the preceding claims, wherein the linking polypeptide is derived from a CD8a hinge domain or a functional variant thereof.

22. The chimeric receptor of any one of the preceding claims, wherein the linking polypeptide is derived from an CD28 hinge domain or a functional variant thereof.

23. The chimeric receptor of any one of the preceding claims, wherein the linking polypeptide comprises an amino acid sequence having at least 80% sequence identity to SEQ ID NO: 3.

24. The chimeric receptor of any one of the preceding claims, wherein the one or more ligand-inducible proteolytic cleavage sites comprises a γ secretase cleavage site.

25. The chimeric receptor of any one of the preceding claims, wherein the TMD comprises an amino acid sequence having at least 80% sequence identity to SEQ ID NO: 4.

26. The chimeric receptor of any one of the preceding claims, further comprising a stop-transfer-sequence (STS) positioned between the TMD and the ICD.

27. The chimeric receptor of any one of the preceding claims, wherein the stop-transfer-sequence comprises an amino acid sequence having at least 80% sequence identity to SEQ ID NO: 5.

28. The chimeric receptor of any one of the preceding claims, wherein the signaling molecule comprises a class 1 or a class 3 human membrane protein.

29. The chimeric receptor of any one of the preceding claims, wherein the signaling molecule is selected from the group consisting of CD28, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, 4-1BB, OX40, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-PlA, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC.

30. The chimeric receptor of any one of the preceding claims, wherein the signaling molecule is selected from the list consisting of OX40, ICOS, 4-1BB, CTLA4, CD28, CD30, CD2, CD27, and CD226.

31. The chimeric receptor of any one of the preceding claims, wherein the activation domain comprises one or more immunoreceptor tyrosine-based activation motifs (ITAMs).

32. The chimeric receptor of any one of the preceding claims, wherein the one or more ITAMs are derived from CD3ζ, CD3σ, CD3/, and CD3ε.

33. The chimeric receptor of any one of the preceding claims, wherein the one or more ITAMs have at least about 80, 85, 90, 95, 96, 97, 98, 99, or 100% sequence identity to a CD3ζ ITAM.

34. The chimeric receptor of any one of the preceding claims, wherein the transcriptional regulator comprises a transcriptional activator or a transcriptional repressor.

35. The chimeric receptor of any one of the preceding claims, wherein the transcriptional regulator further comprises a nuclear localization sequence (NLS) derived from a protein selected from the group consisting of Gal4, tetR, ZFHD1, and HAP1, and wherein the transcriptional regulator comprises a transactivation domain derived from a protein selected from the group consisting of VP64, VP65, KRAB, and VP16.

36. The chimeric receptor of any one of the preceding claims, comprising an amino acid sequence having at least 80% sequence identity to any one of SEQ ID NOS: 15-32, 34-44, 47-48, 54-58, and 63-68.

37. The chimeric receptor of any one of the preceding claims, further comprising a signal sequence, a detectable label, a tumor-specific cleavage site, a disease-specific cleavage site, or a combination thereof.

38. A recombinant nucleic acid comprising a nucleotide sequence encoding the chimeric receptor of any one of the preceding claims.

39. The recombinant nucleic acid of claim 38, wherein the nucleotide sequence is incorporated into an expression cassette or an expression vector.

40. The recombinant nucleic acid of claim 39, wherein the expression vector is a viral vector.

41. The recombinant nucleic acid of claim 40, wherein the viral vector is a lentiviral vector, an adeno virus vector, an adeno-associated virus vector, or a retroviral vector.

42. A recombinant cell comprising the chimeric receptor and/or the recombinant nucleic acid according to any one of the preceding claims.

43. The recombinant cell of claim 42, wherein the recombinant cell is a eukaryotic cell.

44. The recombinant cell of claim 43, wherein the eukaryotic cell is a mammalian cell.

45. The recombinant cell of claim 44, wherein the mammalian cell is an immune cell, a neuron, an epithelial cell, and endothelial cell, or a stem cell.

46. The recombinant cell of claim 45, wherein the immune cell is a B cell, a monocyte, a natural killer cell, a basophil, an eosinophil, a neutrophil, a dendritic cell, a macrophage, a regulatory T cell, a helper T cell, a cytotoxic T cell, or other T cell.

47. The recombinant cell of any one of the preceding claims, comprising:
a) a first chimeric receptor and a second chimeric receptor according to any one of claims 1 to 37; and/or
b) a first nucleic acid and a second nucleic acid according to any one of claims 38 to 41;
wherein the first chimeric receptor and the second chimeric receptor do not have the same sequence, and/or the first nucleic acid or the second nucleic acid do not have the same sequence.

48. The recombinant cell of claim 47, wherein the first chimeric receptor modulates the expression and/or activity of the second chimeric receptor.

49. The recombinant cell of any one of claims 42-48, further comprising an expression cassette encoding a protein operably linked to a promoter, wherein expression of the protein is modulated by the transcriptional regulator.

50. The recombinant cell of claim 49, wherein the protein is heterologous to the cell.

51. The recombinant cell of claim 49, wherein the promoter is a yeast GAL4 promoter.

52. The recombinant cell of any one of claims 49-51, wherein the protein is a cytokine, a cytotoxin, a chemokine, an immunomodulator, a pro-apoptotic factor, an anti-apoptotic factor, a hormone, a differentiation factor, a de-differentiation factor, an immune cell receptor (e.g., a TCR or CAR), or a reporter.

53. A method for making the recombinant cell according to any one of claims 42 to 52, comprising:
a) providing a cell capable of protein expression;
b) contacting the provided cell with a recombinant nucleic acid according to any one of claims 38 to 41 into the provided cell.

54. The method of claim 53, wherein the cell is obtained by leukapheresis performed on a sample obtained from a subject, and the cell is contacted ex vivo.

55. The method of claim 54, wherein the recombinant nucleic acid is encapsulated in a viral capsid or a lipid nanoparticle.

56. A pharmaceutical composition comprising a pharmaceutically acceptable carrier, and one or more of the following:
a) the recombinant nucleic acid according to any one of claims 38-41; and
b) the recombinant cell according to any one of claims 42-52.

57. The pharmaceutical composition of claim 56, wherein the composition comprises a recombinant nucleic acid according to any one of claims 38 to 41, and a pharmaceutically acceptable carrier.

58. The pharmaceutical composition of claim 56, wherein the recombinant nucleic acid is encapsulated in a viral capsid or a lipid nanoparticle.

59. A system for modulating an activity of a cell, inhibiting a target cancer cell, or treating a health condition in an individual in need thereof, the system comprising one or more of the following:
a) a chimeric receptor according to any one of claims 1 to 37;
b) a recombinant nucleic acid according to any one of claims 38 to 41;
c) a recombinant cell according to any one of claims 42 to 52; and
d) a pharmaceutical composition according to any one of claims 56 to 58.

60. A method for modulating an activity of a cell, the method comprising:
a) providing a recombinant cell according to any one of claims 42 to 52; and
b) contacting the recombinant cell with a selected ligand, wherein binding of the selected ligand to the extracellular ligand-binding domain induces cleavage of a ligand-inducible proteolytic cleavage site and releases the transcriptional regulator, wherein the released transcriptional regulator modulates an activity of the recombinant cell.

61. The method of claim 60, the contacting is carried out in vivo, ex vivo, or in vitro.

62. The method of any one of the preceding claims, wherein the activity of the cell to be modulated is selected from the group consisting of: expression of a selected gene, proliferation, apoptosis, non-apoptotic death, differentiation, dedifferentiation, migration, secretion of a molecule, cellular adhesion, and cytolytic activity.

63. The method of any one of the preceding claims, wherein the released transcriptional regulator modulates expression of a gene product of the cell.

64. The method of any one of the preceding claims, wherein the released transcriptional regulator modulates expression of a heterologous gene product.

65. The method of any one of the preceding claims, wherein the gene product of the cell is selected from the group consisting of chemokine, a chemokine receptor, a chimeric antigen receptor, a cytokine, a cytokine receptor, a differentiation factor, a growth factor, a growth factor receptor, a hormone, a metabolic enzyme, a pathogen-derived protein, a proliferation inducer, a receptor, an RNA guided nuclease, a site-specific nuclease, a T cell receptor, a toxin, a toxin derived protein, a transcriptional regulator, a transcriptional activator, a transcriptional repressor, a translational regulator, a translational activator, a translational repressor, an activating immuno-receptor, an antibody, an apoptosis inhibitor, an apoptosis inducer, an engineered T cell receptor, an immuno-activator, an immuno-inhibitor, and an inhibiting immuno-receptor.

66. The method of any one of the preceding claims, wherein the released transcriptional regulator modulates differentiation of the cell, and wherein the cell is an immune cell, a stem cell, a progenitor cell, or a precursor cell.

67. A method for inhibiting an activity of a target cell in an individual, the method comprising administering to the individual an effective number of the recombinant cells according to any one of claims 42 to 52, wherein the recombinant cells inhibit an activity of the target cell in the individual.

68. The method of claim 67, wherein the target cell is a pathogenic cell.

69. The method of claim 68, wherein the pathogenic cell is a cancer cell.

70. The method of any one of the claims 63 to 69, wherein the target cell is an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-Cell lymphoma cell, a renal cancer cell, a sarcoma cell, a stomach cancer cell, a carcinoma cell, a mesothelioma cell, or a sarcoma cell.

71. A method for the treatment of a health condition in an individual in need thereof, the method comprising administering to the individual a first therapy comprising an effective number of the recombinant cell according to any one of claims 42 to 52, wherein the recombinant cell treats the health condition in the individual.

72. The method of claim 71, further comprising administering to the individual a second therapy.

73. The method of claim 72, wherein the second therapy is selected from the group consisting of chemotherapy, radiotherapy, immunotherapy, hormonal therapy, and toxin therapy.

74. The method of any one of claims 71-73, wherein the first therapy and the second therapy are administered together in the same composition or in separate compositions.

75. The method claim 74, wherein the first therapy and the second therapy are administered at the same time.

76. The method of any one of claims 71-75, wherein the first therapy and the second therapy are administered sequentially.

77. The method of claim 76, wherein the first therapy is administered before the second therapy.

78. The method of claim 76, wherein the first therapy is administered after the second therapy.

79. The method of claim 76, wherein the first therapy and the second therapy are administered in rotation.

80. The use of one or more of the following for the treatment of a health condition:
a) a chimeric receptor according to any one of claims 1 to 37;
b) a recombinant nucleic acid according to any one of claims 38 to 41;
c) a recombinant cell according to any one of claims 42 to 52; and
d) a composition according to any one of claims 56 to 58.

81. The use of the invention of any one of the preceding claims for the manufacture of a medicament for the treatment of a health condition.

82. The use of claim 80 or 81, wherein the health condition is cancer.

83. The use of claim 82, wherein the cancer is a solid tumor, a soft tissue tumor, or a metastatic lesion.
